SEQUENCE LISTING (85 sequences)

SEQ ID NO: 1 - protein, 472 aa, Bacillus subtilis 168
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 2 - protein, 472 aa, Bacillus subtilis ATCC6633
MERKTVLVIADLGGCPPHMFYKSAAEKYNLVSFIPRPFAITASHAALIEKYSVAVIKDKDYFQSLADFEHPDSIYWAHEDHDKPEEEVVEQIVKVAQMFEADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVTFSAAAPGTSVDLTLFEAFNSIAAFELKGSNSQDVAESIRQIQQHAKLTAKYVLPV

SEQ ID NO: 3 - protein, 472 aa, Bacillus subtilis IAM1213
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDLPDGLLKGDTEIVSFSAAAPGTSVDLTLFEAFNSIAAFELKGSNSQDVAESIRQIQQHAKLTAKYVLPV

SEQ ID NO: 4 - protein, 472 aa, Bacillus subtilis IAM1107
MERKTVLVIADLGGCPPHMFYKSAAEKYNLVSFIPRPFAITASHAALIEKYSVAVVKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEVKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIFSFSAAAPGTSVDLTLFEAFNSIAAFELKGSNSQDVAESIRQIQQHAKLTAKYVLPV

SEQ ID NO: 5 - protein, 472 aa, Bacillus subtilis IAM1214; identical to SEQ ID NO: 2

SEQ ID NO: 6 - protein, 472 aa, Bacillus subtilis ATCC21555
MERKTVLVIADLGGCPPHMFYKSAAEKYNLVSFIPRPFAITASHAALIEKYSIAVIKDKDYFKSLADFEHPDSIYWAHEDHDKPEEEVVEEIVKVADMFGVDAITTNNELFIAPMAKACKRLGLRGAGVQAAENARDKNKMRAAFNRAGVKSIKNKRVTTLEDFRAALQEIGTPLILKPTYLASSIGVTLIKEMETAEAEFNRVNEYLKSINVPKAVTFEAPFIAEEFLQGEYDDWYETSGYSDYISIEGIMADGEYFPVAIHDKTPQIGFTETAHITPSILDDDAKRKIVEAAKKANEGLGLENCATHTEIKLMKNREAGLIESAARFAGWNMIPNIKKVFGVDMAQLLLDVLCYGKEADLPKGLLEQEPCYVADCHLYPQHFKENGQLPETVVDFVIESIEIPDGVLKGDTELVSFSAAEAGTSVDLRLFEAFNSIAAFELKGSNSNDVAESIKQIQQQAKLTAKYALSV

SEQ ID NO: 7 - protein, 472 aa, Bacillus amyloliquefaciens IFO3022
MERKTVLVIADLGGCPPHMFYKSAAEKYNLVSFIPRPFAITASHAALIEKYSVAVIKDKDYFKSLADFEHPDSIYWAHEDHDKPEEEVVEEIVKVAGMFAVDAITTNNELFIAPMAKACERLGLRGAGVQAAENARDKNKMRAAFNRAGVKSIKNRRVTTLEDFRAALQEIGTPLILKPTYLASSIGVTLIKERETAEAEFNRVNEYLKSINVPKAVTFEAPFIAEEFLQGEYDDWYETSGYSDYISIEGIMADGEYFPVAIHDKTPQIGFTETSHITPSILDDDAKRKIVEAAKKANEGLGLENCATHTEIKLMKNREAGLIESAARFAGWNMIPNIKKVFGVDMAQLLLDVLCFGKEADLPKGLLEQEPCYVADCHLYPQHFKENGQLPETAVDFVIESIDIPDGVLKGDTEIVSFSAAEAGTSVDLRLFEAFNSIAAFELKGSNSGDVAESIKQIQQQAKLTAKYALPV

SEQ ID NO: 8 - protein, 476 aa, Bacillus pumilus NRRL B-12025
VLSLSKKTVLVIADLGGCPPHMFYESVAASYHIVSYIPRPFAITKGHAELIEKYSIAVIKDRDYFETHPSFEHPDSIYWAHDDYPKSEEEVVEDFIRVASFFKADAITTNNELFIAPMAKAAERLGLRGAGVKAAEMARDKSQMRAAFNASGVKAVKTQPVTTLSDFQQAIESIGTPLILKPTYLASSIGVTLFHDKAGSDDLFLQVQSYLETIPVPDAVTYEAPFVAETYLEGAYEDWYEDEGYADYVSVEGLVVEGEYLPFVIHDKTPQIGFTETAHITPTILDNEAKQIIIEAARKANEGLGLEHCATHTEIKLMKNRETGLIEAAARFAGWNMIPNIKKVFGVDMAKLLIDVLVDGKKAVLPKQLLSGHTFYVADCHLYPQHFKESGLIPPEATHITIDHVSIPQEAFVGDTAIVSQSFPAKGTIVDLELFEAFNGIVSLELKGSSSQDVAASIRNIQKQATIQLMDELVKG

SEQ ID NO: 9 - DNA, 1416 bp, Bacillus subtilis 168
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgaacaccctgattccatttattgggcgcatgaagatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggactgcaaaattgcgcaacacatacagagatcaagctaatgaaaaacagagaaccgggtttaatagagtcggcagccagatttgccggctggaatatgatccccaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccaaattcctgaaactgctgaggatttggtcattgaagcgatcgatattccggacgggcttttaaaaggggatactgaaatcgtttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 10 - DNA, 1416 bp, Bacillus subtilis ATCC6633
atggagagaaaaacagtattggtcatcgctgatcttggaggctgcccgccgcacatgttttataaaagcgctgctgaaaaatataacctggttagctttattccgagaccttttgcaataacagcctcccatgcagcactgattgaaaaatactcggtcgcggtcataaaagataaagactattttcagagcttagctgattttgagcatcccgattcaatttattgggcgcatgaggatcatgacaagcctgaagaagaggttgtcgagcaaatcgtcaaggttgcccaaatgtttgaggcggacgccatcacaacaaacaatgaattattcattgccccgatggcgaaagcctgtgaacgccttggcctgaggggcgccggagtgcaggcagcggaaaatgccagagataaaaataaaatgagggacgcttttaataaggcgggagtcaaatcgatcaaaaacaaacgagtcacaactcttgaggattttcgtgctgcacttgaagagatcggcacacctctaatcttaaagcctacatacttagcgagttcaatcggcgtaacgctgattaccgacacggagacggcagaagatgaatttaacagagtcaatgactacctgaaatcgattaacgtgccgaaggcggtcacatttgaagcaccgtttattgctgaggaatttttacagggtgagtacggagactggtatcaaacagaagggtactccgactatatcagcatagaaggcattatggcagatggtgagtattttccgatcgccattcatgacaaaacgccgcaaattggatttacagagacatcacatattacgccatccattctggatgaagaggcgaaaaagaaaattgtcgaagcggctaaaaaggcaaatgaagggcttggactgcaaaattgcgcaacacatacagaaatcaagctaatgaaaaacagagaaccgggtttaatagagtcggctgccagattcgcaggctggaatatgattcctaacattaaaaaggttttcggccttgatatggcgcaattattattagatgttctctgtttcggaaaagatgctgatctgccggacgggttattggatcaagagccttactatgttgctgactgccatctgtaccctcagcatttcaaacaaaatggccagatccctgaaactgccgaggatttggtaatcgaagcgatcgatattccggatgggcttttgaagggtgatacagaaatcgttactttttcggctgcggcaccaggaacatcagttgatttgacactgtttgaagccttcaactccattgctgcatttgaactgaaaggcagcaattcacaggatgtggctgaatcaatcagacaaattcagcagcatgcgaagctgacggcaaagtatgtgctgccagta

SEQ ID NO: 11 - DNA, 1416 bp, Bacillus subtilis IAM1213
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgactccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactctcgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaaggtcttggcctgcaaaattgcgcaacacatacagagatcaagctaatgaaaaatagagaaccgggtttaattgaatcggcagccagattcgccggctggaatatgatccccaatattaaaaaggtctttggccttgatatggcgcaattattattagatgtcctttgtttcggaaaagacgccgatctgccggacggattattggatcaagagccttattatgttgccgactgccatttgtacccgcaacatttcaaacaaaatggccagattccagaaactgctgaggatttggtcattgaagcgatcgatctgcctgacgggcttttaaaaggggatactgagatcgtttctttttcggccgcagcaccaggaacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 12 - DNA, 1416 bp, Bacillus subtilis IAM1107
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcgtaaaagataaagactattttaagagtttagctgattttgagcatcctgactccatttattgggcgcatgaggatcataacaagcctgaggaagaggtcgtcgagcaaatcgtcaaggttgccgaaatgttcggggcggatgccatcacaacaaacaatgaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagaggtcaagctaatgaaaaacagagaaccgggtttaattgaatcggcagccagatttgccggctggaatatgatccctaacattaaaaaggttttcggccttgatatggcgcaattattattagatgtcctctgtttcggaaaagatgccgatctgccggacggattattggatcaagagccttactatgtcgccgactgccatttgtacccgcagcatttcaaacaaaatggccagattccagaaaccgctgaggatttggtcattgaagcgatcgatattccggacgggcttttaaaaggggatactgaaatcttttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 13 - DNA, 1416 bp, Bacillus subtilis IAM1214; identical to SEQ ID NO: 10

SEQ ID NO: 14 - DNA, 1416 bp, Bacillus subtilis ATCC21555
atggagagaaaaacagtattggttatcgctgatcttgggggctgcccgccgcatatgttttacaaaagcgcagccgaaaaatacaacctcgtcagctttattccgagaccctttgcaattacagcctctcatgcggccttaattgaaaaatactcgattgcggtcattaaagataaagactattttaagagtctggctgattttgaacatcccgattcgatttattgggctcatgaagatcatgacaaacctgaggaagaagtcgtcgaagaaatcgtgaaagtggccgacatgtttggggttgacgccattacgaccaacaatgaactgtttatcgctccgatggcaaaagcgtgtaaacgtctcggcctgcggggagcgggcgtacaggccgctgaaaacgccagagataaaaataaaatgagagccgccttcaaccgggccggcgtcaaatccatcaaaaacaaacgggtgacgaccctggaagatttccgcgccgcgcttcaggaaatcggaacgccgcttattctgaagcctacatatctggcaagctcgatcggcgtgacgcttattaaagagatggaaacggccgaagctgaattcaacagagtcaatgagtacttgaaatcgattaatgtaccgaaagcggtgacgtttgaagcgccgtttatcgcggaagaattcttgcagggcgagtatgatgactggtacgaaacaagcggttattccgactatatcagcatcgaaggcatcatggccgacggagaatacttccccgttgcgatccatgataaaacaccgcaaatcggattcacggagacagcgcatattacgccgtccatcctggatgatgacgccaagcggaaaatcgtcgaagctgccaagaaggcgaatgaaggactcggcctcgaaaactgtgcaacgcatacagaaataaaattaatgaaaaaccgggaagccggactgattgagtcagcggccagattcgcgggatggaatatgattccgaatattaaaaaggtcttcggcgttgatatggcgcagctattattggatgttctctgttacggaaaagaagctgatctgccgaaaggattattggagcaggagccatgctatgtcgcagactgccacttgtatcctcagcatttcaaagagaacggccagctgcctgagacggttgtcgatttcgtcattgaaagcattgaaattcctgacggcgtcttaaagggagacactgaactcgtttctttctcagcggctgaggcgggtacgtcagtggatctgcggctgttcgaagcgttcaacagcattgcggcgtttgagctgaaaggaagcaattcgaacgacgtggccgaatcaatcaaacaaattcagcagcaggcgaagctgactgcaaagtatgcgttatcggta

SEQ ID NO: 15 - DNA, 1416 bp, Bacillus amyloliquefaciens IFO3022
atggagagaaaaacagtattggttatcgctgaccttgggggatgcccgccgcatatgttttacaaaagcgcagccgaaaaatacaacctcgtcagctttattccgagaccttttgcaattacagcctctcatgcggcattaattgaaaaatactcggtcgcggtcataaaagataaagactattttaagagtctggctgattttgagcatcccgattcgatttactgggctcatgaagatcatgacaaacctgaggaagaagtagtcgaagaaatcgtcaaggtggccggcatgttcgcggttgacgccattacgaccaacaatgaactgtttatcgctccgatggcaaaagcgtgtgaacgtctcggcctgcggggagcgggcgtacaggccgctgaaaatgccagagataaaaacaaaatgagagccgctttcaaccgggccggcgtcaagtctatcaaaaacagacgggtgacgacgctggaagatttccgcgccgcgcttcaggaaatcggaacgccgctcattctgaagcctacatatctggcgagctccatcggcgtgacgctcatcaaagagagggaaacggccgaagccgaatttaacagagtcaatgaatacctgaagtcgatcaacgtaccgaaagcggtcacgtttgaagcgccgtttatcgcggaagaatttttgcagggcgagtatgacgactggtacgaaacaagcggttattccgactatatcagcatagaaggcatcatggccgacggagaatacttccctgtcgcaattcatgataaaacaccgcaaatcggattcacggagacatcgcatattacgccgtccatcctggatgatgacgcgaagcggaaaatcgtcgaagcagccaaaaaggcgaatgaaggactcggcctcgaaaactgcgcaacccatacagagattaaattaatgaaaaaccgggaagccggactgattgaatcagcggcacgatttgcgggctggaacatgattccgaatattaaaaaggtcttcggcgtcgatatggcgcagctgttattggatgttctctgtttcggaaaagaagccgatctgccgaaaggattattggagcaggagccgtgctatgtcgccgactgccacttgtatcctcagcatttcaaagagaacggccagctgcctgagacggctgtcgatttcgtcattgaaagcattgacattcccgacggcgtcttaaagggagacaccgaaatcgtttctttctcggcggccgaggcgggtacatccgtggatctgcggctgttcgaagcgttcaacagcattgcggcgttcgagctgaaaggaagcaattcgggtgacgtggccgaatcaatcaaacaaattcagcagcaggcgaagctgactgcaaagtatgcgttaccggta

SEQ ID NO: 16 - DNA, 1428 bp, Bacillus pumilus NRRL B-12025
gtgctttcattgagtaaaaaaactgtacttgtcattgctgacttaggagggtgcccgccccatatgttttatgaaagcgtggcggcatcataccatatcgtttcttatatcccaagaccctttgcgattacaaagggacatgccgagctaatcgaaaaatactccattgccgtcatcaaagaccgtgattattttgagacacacccttcttttgaacaccctgattctatttactgggcacatgatgattatccaaaatcagaagaagaagttgtggaagacttcattcgagtagcttcctttttcaaagcagatgcaatcacgaccaataatgaattattcattgcaccgatggcaaaggccgctgaacgtcttgggctacgaggtgccggtgtcaaggcagccgaaatggcgcgtgataaaagccaaatgagggctgcattcaatgcctctggcgtcaaagcggtgaaaactcagcctgtcacgactttatctgatttccaacaagccattgagtctatcggaacaccgctcattttaaagcctacatatttagccagttctattggcgtcaccttgtttcatgacaaagccggaagtgatgacttgtttttacaagtacaatcgtatttggaaaccataccagtcccagacgctgtcacgtatgaagcaccgtttgtcgctgaaacatatttagagggtgcttacgaagattggtatgaagacgaaggatatgctgattatgtcagtgtagaagggctggtcgtagagggcgaatatctcccttttgtcatacatgataaaacccctcaaatcggctttacagaaacggctcatatcactccgacgatcttagacaatgaagccaagcaaatcatcattgaagcagcaaggaaggcaaatgaagggctaggtcttgaacattgtgcaacccatacagaaatcaaactcatgaaaaatcgagaaactggactgatcgaggcagcggctcgattcgctggctggaatatgatcccgaatattaaaaaagtctttggcgtcgatatggcgaagctattgattgatgtattagttgatggtaaaaaggctgtactgccaaaacagctgctttctggacatacattttatgtagcggactgccacctgtaccctcagcattttaaagagagtgggcttatcccgcctgaagccacacatattaccattgatcatgtgtctattccgcaggaagcattcgttggagatactgcgattgtcagtcaatcattccctgccaaagggactattgtggatcttgaattatttgaagcttttaatggaatcgtatctcttgaattaaaaggatcatcctcacaagatgttgccgcgtccatccgcaacattcagaaacaggcaacgattcagttaatggatgaattagtgaaggga

SEQ ID NO: 17 - protein, 93 aa, Bacillus subtilis 168
GAGVQAAENARDKNKMRDAFNKAGVKSIKNKRVTTLEDFRAALEEIGTPLILKPTYLASSIGVTLITDTETAEDEFNRVNDYLKSINVPKAVT

SEQ ID NO: 18 - DNA, 279 bp, Bacillus subtilis 168
ggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacg

SEQ ID NO: 19 - DNA, 30 bp, Artificial Sequence
  Description of Artificial Sequence: Synthetic DNA
attctcgagtagagaaggagtgttttacat

SEQ ID NO: 20 - DNA, 30 bp, Artificial Sequence
  Description of Artificial Sequence: Synthetic DNA
ttaggatcctcatactggcagcacatactt

SEQ ID NO: 21 - DNA, 24 bp, Artificial Sequence
  Description of Artificial Sequence: Synthetic DNA
caagaattctcatgtttgacagct

SEQ ID NO: 22 - DNA, 28 bp, Artificial Sequence
  Description of Artificial Sequence: Synthetic DNA
taactcgagattccctttttacgtgaac

SEQ ID NO: 23 - DNA, 27 bp, Artificial Sequence
  Description of Artificial Sequence: Synthetic DNA
ttaaccatggagagaaaaacagtattg

SEQ ID NO: 24 - DNA, 30 bp, Artificial Sequence
  Description of Artificial Sequence: Synthetic DNA
atatggatcctactggcagcacatactttg

SEQ ID NO: 25 - DNA, 21 bp, Artificial Sequence
  Description of Artificial Sequence: Synthetic DNA
caccgcagacggaggatacac

SEQ ID NO: 26 - DNA, 22 bp, Artificial Sequence
  Description of Artificial Sequence: Synthetic DNA
cggacgtcacccaataatcgtg

SEQ ID NO: 27 - DNA, 23 bp, Artificial Sequence
  Description of Artificial Sequence: Synthetic DNA
ccgatggcraaagcstgtraacg

SEQ ID NO: 28 - DNA, 21 bp, Artificial Sequence
  Description of Artificial Sequence: Synthetic DNA
cggcagatcrgcdtcttttcc

SEQ ID NO: 29 - DNA, 26 bp, Artificial Sequence
  Description of Artificial Sequence: Synthetic DNA
gctaggtcttgaacattgtgcaaccc

SEQ ID NO: 30 - DNA, 23 bp, Artificial Sequence
  Description of Artificial Sequence: Synthetic DNA
ggtgttccgatagactcaatggc

SEQ ID NO: 31 - DNA, 44 bp, Artificial Sequence
  Description of Artificial Sequence: Synthetic DNA
catgccatggagaaaaaaactgtacttgtcattgctgacttagg

SEQ ID NO: 32 - DNA, 38 bp, Artificial Sequence
  Description of Artificial Sequence: Synthetic DNA
cgcggatcccttcactaattcatccattaactgaatcg

SEQ ID NO: 33 - protein, 12 aa, Artificial Sequence
  UNSURE (3): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (4): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (9): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (10): Xaa is Leu, Ile, Val, Met or Ala
  UNSURE (11): Xaa is Glu, Ser or Ala
  UNSURE (12): Xaa is Gly, Ser or Ala
  Description of Artificial Sequence: Synthetic Peptide
HGXXGQDGXXXX

SEQ ID NO: 34 - protein, 28 aa, Artificial Sequence
  UNSURE (1): Xaa represents Leu Ile or Val
  UNSURE (2): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (3): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (4): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (5): Xaa is Gly or Ala
  UNSURE (6): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (7): Xaa is Gly, Ser, Ala, Ile or Val
  UNSURE (9): Xaa is Leu, Ile, Val, Met, Cys or Ala
  UNSURE (11): Xaa is Leu, Ile, Val, Met, Phe or Ala
  UNSURE (12): Xaa is Leu, Ile, Val, Met, Phe or Ala
  UNSURE (13): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (14): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (15): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (16): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (17): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (18): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (19): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (20): Xaa is Leu, Ile or Val
  UNSURE (21): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (23): Xaa is Leu, Ile, Val, Ala or Pro
  UNSURE (25): Xaa is Ser, Thr or Pro
  UNSURE (26): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (28): Xaa is Gly or Ala
  Description of Artificial Sequence: Synthetic Peptide
XXXXXXXRXDXXXXXXXXXXXQXNXXPX

SEQ ID NO: 35 - protein, 30 aa, Artificial Sequence
  UNSURE (1): Xaa is Leu Ile or Val
  UNSURE (2): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (3): Xaa is from Ala, Arg, Asn, Asp, Cys, Glu, Gln, Gly, His, Ile, Leu, Lys, Met, Phe, Pro, Ser, Thr,Trp, Tyr or Val
  UNSURE (4): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (5): Xaa is Gly or Ala
  UNSURE (6): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (7): Xaa is Gly, Ser, Ala, Ile or Val
  UNSURE (9): Xaa is Leu, Ile, Val, Met, Cys or Ala
  UNSURE (11): Xaa is Leu, Ile, Val, Met, Phe or Ala
  UNSURE (12): Xaa is Leu, Ile, Val, Met, Phe or Ala
  UNSURE (13): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (14): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (15): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (16): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (17): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (18): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (19): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (20): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (21): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (22): Xaa is Leu, Ile or Val
  UNSURE (23): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (25): Xaa is Leu, Ile, Val, Ala or Pro
  UNSURE (27): Xaa is Ser, Thr or Pro
  UNSURE (28): Xaa is Ala, Arg, Asn, Asp, Cys, Glu, Gln, Gly, His, Ile, Leu, Lys, Met, Phe, Pro, Ser, Thr, Trp, Tyr or Val
  UNSURE (30): Xaa is Gly or Ala
  Description of Artificial Sequence: Synthetic Peptide
XXXXXXXRXDXXXXXXXXXXXXXQXNXXPX

SEQ ID NO: 36 - DNA, 1416 bp, Bacillus subtilis ATCC 15245 and Bacillus subtilis IAM 1033
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgattccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagagatcaagctaatgaaaaacagagaaccgggtttaatagagtcggcagccagattcgcaggctggaatatgattcctaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccagattccagaaaccgctgaggatttggtcattgaagcgatcgatattccggacgggcttttaaaaggggatactgaaatcgtttcattttcagccgcagcaccaggcacttcagttgatttgacattgtttgaagctttcaattccattgctgcatttgaactgaaaggcagtaattcacaggatgtggctgaatcaatcagacaaattcagcagcatgcaaagctgacggcaaagtatgtgctgccagta

SEQ ID NO: 37 - protein, 239 aa, Streptomyces noursei IFO15452
MLAGLVPAPDHGMREEILGDRSRLIRQRGEHALIGISAGNSYFSQKNTVMLLQWAGQRFERTDVVYVDTHIDEMLIADGRSAQEAERSVKRTLKDLRRRLRRSLESVGDHAERFRVRSLSELQETPEYRAVRERTDRAFEEDAEFATACEDMVRAVVMNRPGDGVGISAEHLRAGLNYVLAEAPLFADSPGVFSVPSSVLCYHIDTPITAFLSRRETGFRAAEGQAYVVVRPQELADAA

SEQ ID NO: 38 - protein, 239 aa, Streptomyces alborus IFO15452
MLAGLVPALDHSMREEILGNRGRKIRQRGEHALIGISAGNSYFSQKNVTMLLQWAGQHFERTDVVYVDTHIDDMLMADGRSAQEAEKSVKRTLKDLRRRLRRSLESVGDHSERFRVRSLSEIQETPEYRAARESTDRAFREDGEFATVCEEMVRAVVMNRPGDGVDISEEHLRAGLNYVLAEAPLFADSPGVFSVPSSVLCYHIPTPVSTFLAHRETGFQAAQGQAYVVVRPQELADAA

SEQ ID NO: 39 - DNA, 717 bp, Streptomyces noursei IFO15452
atgcttgcaggcttagttcccgcgccggaccacggaatgcgggaagaaatacttggcgaccgcagccgattgatccggcaacgcggtgagcacgccctcatcggaatcagtgcgggcaacagttatttcagccagaagaacaccgtcatgctgctgcaatgggccgggcagcgtttcgagcgcaccgatgtcgtctatgtcgacacccacatcgacgagatgctgatcgccgacggccgcagcgcgcaggaggccgagcggtcggtcaaacgcacgctcaaggatctgcggcgcagactccggcgctcgctggagagcgtgggcgaccacgccgagcggttccgtgtccggtccctgtccgagctccaggagacccctgagtaccgggccgtacgcgagcgcaccgaccgggccttcgaggaggacgccgaattcgccaccgcctgcgaggacatggtgcgggccgtggtgatgaaccggcccggtgacggcgtcggcatctccgcggaacacctgcgggccggtctgaactacgtgctggccgaggccccgctcttcgcggactcgcccggagtcttctccgtcccctcctcggtgctctgctaccacatcgacaccccgatcacggcgttcctgtcccggcgcgagaccggtttccgggcggccgagggacaggcgtacgtcgtcgtcaggccccaggagctggccgacgcggcc

SEQ ID NO: 40 - DNA, 717 bp, Streptomyces alborus IFO15452
atgcttgcaggcttagttcccgcgctggaccacagcatgcgggaagaaatacttggcaatcgcggccgaaagatccggcaacgcggtgagcacgctctcattggaatcagtgcgggcaacagttatttcagccagaagaacgtcaccatgctgctgcaatgggccgggcagcatttcgagcgcacggatgtcgtctacgtggacacgcacatcgacgacatgctgatggcggacggccgcagcgcgcaggaagccgagaagtcggtcaagcgcacgctcaaggatctgcggcgcaggctgcggcgctcgttggaaagcgtgggcgaccacagcgagcggttccgcgtccggtccctgtccgagatccaggagacccctgagtaccgggccgcacgcgagtccaccgaccgggccttccgcgaggacggcgagttcgccaccgtctgcgaggagatggtgcgcgccgtggtgatgaaccggcccggtgacggcgtcgacatctcggaggaacacctgcgggccggtctgaactacgtgctcgccgaggccccgctcttcgcggactcgcccggcgtgttctccgtcccctcgtcggtgctctgctaccacatccccaccccggtatcgacgttcctggcccatcgcgagaccggtttccaggcggctcagggtcaggcatacgtcgtcgtcaggccgcaggagctggccgacgcggcc

SEQ ID NO: 41 - DNA, 32 bp, Artificial Sequence
  Description of Artificial Sequence: Synthetic DNA
agagccatgggacttgcaggcttagttcccgc

SEQ ID NO: 42 - DNA, 29 bp, Artificial Sequence
  Description of Artificial Sequence: Synthetic DNA
agagagatctggccgcgtcggccagctcc

SEQ ID NO: 43 - protein, 503 aa, Escherichia coli
MEFSVKSGSPEKQRSACIVVGVFEPRRLSPIAEQLDKISDGYISALLRRGELEGKPGQTLLLHHVPNVLSERILLIGCGKERELDERQYKQVIQKTINTLNDTGSMEAVCFLTELHVKGRNNYWKVRQAVETAKETLYSFDQLKTNKSEPRRPLRKMVFNVPTRRELTSGERAIQHGLAIAAGIKAAKDLGNMPPNICNAAYLASQARQLADSYSKNVITRVIGEQQMKELGMHSYLAVGQGSQNESLMSVIEYKGNASEDARPIVLVGKGLTFDSGGISIKPSEGMDEMKYDMCGAAAVYGVMRMVAELQLPINVIGVLAGCENMPGGRAYRPGDVLTTMSGQTVEVLNTDAEGRLVLCDVLTYVERFEPEAVIDVATLTGACVIALGHHITGLMANHNPLAHELIAASEQSGDRAWRLPLGDEYQEQLESNFADMANIGGRPGGAITAGCFLSRFTRKYNWAHLDIAGTAWRSGKAKGATGRPVALLAQFLLNRAGFNGEE

SEQ ID NO: 44 - protein, 427 aa, Escherichia coli
MTEAMKITLSTQPADARWGEKATYSINNDGITLHLNGADDLGLIQRAARKIDGLGIKHVQLSGEGWDADRCWAFWQGYKAPKGTRKVVWPDLDDAQRQELDNRLMIIDWVRDTINAPAEELGPSQLAQRAVDLISNVAGDRVTYRITKGEDLREQGYMGLHTVGRGSERSPVLLALDYNPTGDKEAPVYACLVGKGITFDSGGYSIKQTAFMDSMKSDMGGAATVTGALAFAITRGLNKRVKLFLCCADNLISGNAFKLGDIITYRNGKKVEVMNTDAEGRLVLADGLIDASAQKPEMIIDAATLTGAAKTALGNDYHALFSFDDALAGRLLASAAQENEPFWRLPLAEFHRSQLPSNFAELNNTGSAAYPAGASTAAGFLSHFVENYQQGWLHIDCSATYRKAPVEQWSAGATGLGVRTIANLLTA

SEQ ID NO: 45 - protein, 485 aa, Escherichia coli
MSELSQLSPQPLWDIFAKICSIPHPSYHEEQLAEYIVGWAKEKGFHVERDQVGNILIRKPATAGMENRKPVVLQAHLDMVPQKNNDTVHDFTKDPIQPYIDGEWVKARGTTLGADNGIGMASALAVLADENVVHGPLEVLLTMTEEAGMDGAFGLQGNWLQADILINTDSEEEGEIYMGCAGGIDFTSNLHLDREAVPAGFETFKLTLKGLKGGHSGGEIHVGLGNANKLLVRFLAGHAEELDLRLIDFNGGTLRNAIPREAFATIAVAADKVDVLKSLVNTYQEILKNELAEKEKNLALLLDSVANDKAALIAKSRDTFIRLLNATPNGVIRNSDVAKGVVETSLNVGVVTMTDNNVEIHCLIRSLIDSGKDYVVSMLDSLGKLAGAKTEAKGAYPGWQPDANSPVMHLVRETYQRLFNKTPNIQIIHAGLECGLFKKPYPEMDMVSIGPTITGPHSPDEQVHIESVGHYWTLLTELLKEIPAK

SEQ ID NO: 46 - protein, 870 aa, Escherichia coli
MTQQPQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 47 - protein, 535 aa, Escherichia coli
MRISLKKSGMLKLGLSLVAMTVAASVQAKTLVYCSEGSPEGFNPQLFTSGTTYDASSVPLYNRLVEFKIGTTEVIPGLAEKWEVSEDGKTYTFHLRKGVKWHDNKEFKPTRELNADDVVFSFDRQKNAQNPYHKVSGGSYEYFEGMGLPELISEVKKVDDNTVQFVLTRPEAPFLADLAMDFASILSKEYADAMMKAGTPEKLDLNPIGTGPFQLQQYQKDSRIRYKAFDGYWGTKPQIDTLVFSITPDASVRYAKLQKNECQVMPYPNPADIARMKQDKSINLMEMPGLNVGYLSYNVQKKPLDDVKVRQALTYAVNKDAIIKAVYQGAGVSAKNLIPPTMWGYNDDVQDYTYDPEKAKALLKEAGLEKGFSIDLWAMPVQRPYNPNARRMAEMIQADWAKVGVQAKIVTYEWGEYLKRAKDGEHQTVMMGWTGDNGDPDNFFATLFSCAASEQGSNYSKWCYKPFEDLIQPARATDDHNKRVELYKQAQVVMHDQAPALIIAHSTVFEPVRKEVKGYVVDPLGKHHFENVSIE

SEQ ID NO: 48 - protein, 339 aa, Escherichia coli
MLQFILRRLGLVIPTFIGITLLTFAFVHMIPGDPVMIMAGERGISPERHAQLLAELGLDKPMWQQYLHYIWGVMHGDLGISMKSRIPVWEEFVPRFQATLELGVCAMIFATAVGIPVGVLAAVKRGSIFDHTAVGLALTGYSMPIFWWGMMLIMLVSVHWNLTPVSGRVSDMVFLDDSNPLTGFMLIDTAIWGEDGNFIDAVAHMILPAIVLGTIPLAVIVRMTRSSMLEVLGEDYIRTARAKGLTRMRVIIVHALRNAMLPVVTVIGLQVGTLLAGAILTETIFSWPGLGRWLIDALQRRDYPVVQGGVLLVATMIILVNLLVDLLYGVVNPRIRHKK

SEQ ID NO: 49 - protein, 300 aa, Escherichia coli
MSQVTENKVISAPVPMTPLQEFWHYFKRNKGAVVGLVYVVIVLFIAIFANWIAPYNPAEQFRDALLAPPAWQEGGSMAHLLGTDDVGRDVLSRLMYGARLSLLVGCLVVVLSLIMGVILGLIAGYFGGLVDNIIMRVVDIMLALPSLLLALVLVAIFGPSIGNAALALTFVALPHYVRLTRAAVLVEVNRDYVTASRVAGAGAMRQMFINIFPNCLAPLIVQASLGFSNAILDMAALGFLGMGAQPPTPEWGTMLSDVLQFAQSAWWVVTFPGLAILLTVLAFNLMGDGLRDALDPKLKQ

SEQ ID NO: 50 - protein, 327 aa, Escherichia coli
MALLNVDKLSVHFGDESAPFRAVDRISYSVKQGEVVGIVGESGSGKSVSSLAIMGLIDYPGRVMAEKLEFNGQDLQRISEKERRNLVGAEVAMIFQDPMTSLNPCYTVGFQIMEAIKVHQGGNKSTRRQRAIDLLNQVGIPDPASRLDVYPHQLSGGMSQRVMIAMAIACRPKLLIADEPTTALDVTIQAQIIELLLELQQKENMALVLITHDLALVAEAAHKIIVMYAGQVVETGDAHAIFHAPRHPYTQALLRALPEFAQDKERLASLPGVVPGKYDRPNGCLLNPRCPYATDRCRAEEPALNMLADGRQSKCHYPLDDAGRPTL

SEQ ID NO: 51 - protein, 334 aa, Escherichia coli
MSTQEATLQQPLLQAIDLKKHYPVKKGMFAPERLVKALDGVSFNLERGKTLAVVGESGCGKSTLGRLLTMIEMPTGGELYYQGQDLLKHDPQAQKLRRQKIQIVFQNPYGSLNPRKKVGQILEEPLLINTSLSKEQRREKALSMMAKVGLKTEHYDRYPHMFSGGQRQRIAIARGLMLDPDVVIADEPVSALDVSVRAQVLNLMMDLQQELGLSYVFISHDLSVVEHIADEVMVMYLGRCVEKGTKDQIFNNPRHPYTQALLSATPRLNPDDRRERIKLSGELPSPLNPPPGCAFNARCRRRFGPCTQLQPQLKDYGGQLVACFAVDQDENPQR

SEQ ID NO: 52 - DNA, 34 bp, Artificial Sequence
  Description of Artificial Sequence: Synthetic DNA
gaagttcctatactttctagagaataggaacttc

SEQ ID NO: 53 - DNA, 1509 bp, Escherichia coli
atggagtttagtgtaaaaagcggtagcccggagaaacagcggagtgcctgcatcgtcgtgggcgtcttcgaaccacgtcgcctttctccgattgcagaacagctcgataaaatcagcgatgggtacatcagcgccctgctacgtcggggcgaactggaaggaaaaccggggcagacattgttgctgcaccatgttccgaatgtactttccgagcgaattctccttattggttgcggcaaagaacgtgagctggatgagcgtcagtacaagcaggttattcagaaaaccattaatacgctgaatgatactggctcaatggaagcggtctgctttctgactgagctgcacgttaaaggccgtaacaactactggaaagtgcgtcaggctgtcgagacggcaaaagagacgctctacagtttcgatcagctgaaaacgaacaagagcgaaccgcgtcgtccgctgcgtaagatggtgttcaacgtgccgacccgccgtgaactgaccagcggtgagcgcgcgatccagcacggtctggcgattgccgccgggattaaagcagcaaaagatctcggcaatatgccgccgaatatctgtaacgccgcttacctcgcttcacaagcgcgccagctggctgacagctacagcaagaatgtcatcacccgcgttatcggcgaacagcagatgaaagagctggggatgcattcctatctggcggtcggtcagggttcgcaaaacgaatcgctgatgtcggtgattgagtacaaaggcaacgcgtcggaagatgcacgcccaatcgtgctggtgggtaaaggtttaaccttcgactccggcggtatctcgatcaagccttcagaaggcatggatgagatgaagtacgatatgtgcggtgcggcagcggtttacggcgtgatgcggatggtcgcggagctacaactgccgattaacgttatcggcgtgttggcaggctgcgaaaacatgcctggcggacgagcctatcgtccgggcgatgtgttaaccaccatgtccggtcaaaccgttgaagtgctgaacaccgacgctgaaggccgcctggtactgtgcgacgtgttaacttacgttgagcgttttgagccggaagcggtgattgacgtggcgacgctgaccggtgcctgcgtgatcgcgctgggtcatcatattactggtctgatggcgaaccataatccgctggcccatgaactgattgccgcgtctgaacaatccggtgaccgcgcatggcgcttaccgctgggtgacgagtatcaggaacaactggagtccaattttgccgatatggcgaacattggcggtcgtcctggtggggcgattaccgcaggttgcttcctgtcacgctttacccgtaagtacaactgggcgcacctggatatcgccggtaccgcctggcgttctggtaaagcaaaaggcgccaccggtcgtccggtagcgttgctggcacagttcctgttaaaccgcgctgggtttaacggcgaagag

SEQ ID NO: 54 - DNA, 1281 bp, Escherichia coli
atgacagaagcgatgaagattaccctctctacccaacctgccgacgcgcgctggggagaaaaagcaacttacagcattaataatgacggcattaccctgcatttgaacggggcagacgatctggggctgatccagcgtgcggcgcgcaagattgacggtctgggcatcaagcatgttcagttaagcggtgaaggctgggatgcggatcgctgctgggcattctggcaaggttacaaagccccgaaaggcacgcgtaaagtggtgtggccggatctggacgatgcccagcgccaggaactggataaccgcctgatgatcatcgactgggtgcgtgacaccatcaacgcaccggcagaagaattgggaccatcgcaactggcacagcgtgctgttgatctgatcagcaacgtcgcgggcgatcgtgtgacttatcggatcaccaaaggcgaagatctgcgtgagcaaggttatatggggctgcacacagtcggacgcggttcagaacgttctccggtattgctggcgctggattacaacccaactggcgataaagaagcgccagtgtacgcgtgcctggtaggtaaaggtatcacttttgactccggcggctacagcatcaaacagactgcgtttatggactcgatgaagtcggacatgggcggcgcggcaacggttaccggggcgctggcatttgccattacgcgcggactgaacaagcgcgtgaagctgttcctctgctgtgcggataacctgattagcggcaatgcgttcaagctgggcgatatcatcacctatcgcaacggtaaaaaagttgaagtgatgaacactgatgccgaagggcgtctggtgcttgccgatggtctgattgatgccagtgcgcagaaaccggaaatgatcattgatgcggcgaccctcaccggggcggcgaaaactgcgctgggtaatgattatcacgcgctgttcagttttgacgatgcgctggccggtcgcttgctggcgagtgccgcgcaggagaacgaaccgttctggcgtctgccgctggcggagttccaccgcagccagctgccgtctaactttgccgaactgaacaataccggaagcgcggcgtatccggcaggcgcgagcacggcggcgggcttcctgtcgcactttgttgagaactatcagcaaggctggctgcatatcgactgctcggcgacttaccgtaaagcgccggttgaacagtggtctgcgggcgctacgggacttggtgtgcgcacgatagctaatctgttaacggcg

SEQ ID NO: 55 - DNA, 1455 bp, Escherichia coli
gtgtctgaactgtctcaattatctccacagccgctgtgggatatttttgccaaaatctgttctattcctcacccgtcctatcatgaagagcaactcgctgaatacattgttggttgggcaaaagagaaaggtttccatgtcgaacgcgatcaggtaggtaatatcctgattcgtaaacctgctaccgcaggtatggaaaatcgtaaaccggtcgtcttacaggcccacctcgatatggtgccgcagaaaaataacgacaccgtgcatgacttcacgaaagatcctatccagccttatattgatggcgaatgggttaaagcgcgcggcaccacgctgggtgcggataacggcattggtatggcctctgcgctggcggttctggctgacgaaaacgtggttcacggcccgctggaagtgctgctgaccatgaccgaagaagccggtatggacggtgcgttcggcttacagggcaactggttgcaggctgatattctgattaacaccgactccgaagaagaaggtgaaatctacatgggttgtgcggggggtatcgacttcacctccaacctgcatttagatcgtgaagcggttccagctggttttgaaaccttcaagttaaccttaaaaggtctgaaaggcggtcactccggcggggaaatccacgttgggctgggtaatgccaacaaactgctggtgcgcttcctggcgggtcatgcggaagaactggatctgcgccttatcgatttcaacggcggcacactgcgtaacgccatcccgcgtgaagcctttgcgaccattgctgtcgcagctgataaagtcgacgtcctgaaatctctggtgaatacctatcaggagatcctgaaaaacgagctggcagaaaaagagaaaaatctggccttgttgctggactctgtagcgaacgataaagctgccctgattgcgaaatctcgcgatacctttattcgtctgctgaacgccaccccgaacggtgtgattcgtaactccgatgtagccaaaggtgtggttgaaacctccctgaacgtcggtgtggtgaccatgactgacaataacgtagaaattcactgcctgatccgttcactgatcgacagcggtaaagactacgtggtgagcatgctggattcgctgggtaaactggctggcgcgaaaaccgaagcgaaaggcgcatatcctggctggcagccggacgctaattctccggtgatgcatctggtacgtgaaacctatcagcgcctgttcaacaagacgccgaacatccagattatccacgcgggcctggaatgtggtctgttcaaaaaaccgtatccggaaatggacatggtttctatcgggccaactatcaccggtccacactctccggatgagcaagttcacatcgaaagcgtaggtcattactggacactgctgactgaactgctgaaagaaattccggcgaag

SEQ ID NO: 56 - DNA, 2610 bp, Escherichia coli
atgactcaacagccacaagccaaataccgtcacgattatcgtgcgccggattaccagattactgatattgacttgacctttgacctcgacgcgcaaaagacggtcgttaccgcggtcagccaggctgtccgtcatggtgcatcagatgctccccttcgtctcaacggcgaagacctcaaactggtttctgttcatattaatgatgagccgtggaccgcctggaaagaagaagagggcgcactggttatcagtaatttgccggagcgttttacgcttaagatcattaatgaaataagcccggcggcgaataccgcgctggaagggctttatcagtcaggcgatgcgctttgcacccagtgtgaagccgaaggtttccgccatattacgtattatctcgaccgcccggacgtgctggcgcgttttaccaccaaaattattgccgataaaatcaaatatcccttcctgctttccaatggtaaccgcgttgcgcaaggcgaactggaaaacggacgccattgggtacagtggcaggacccgttcccgaaaccgtgctacctgtttgcgctggtggcaggcgactttgatgtactgcgcgatacctttaccacgcgttctggtcgcgaagtagcactggagctgtacgtcgatcgcggcaaccttgatcgcgcgccgtgggcgatgacctcgctgaaaaactccatgaaatgggatgaagaacgctttggcctggagtatgacctcgacatctatatgatcgtcgcggtggatttcttcaatatgggcgcaatggagaataaggggctgaatatctttaactccaaatatgtgctggcccgcaccgacaccgccaccgacaaagattacctcgatattgaacgcgttatcggccatgaatatttccataactggaccggtaaccgagtgacctgtcgcgactggttccagctcagcctgaaagaaggtttaaccgtcttccgcgatcaggagttcagctctgaccttggttcccgcgcagttaaccgcatcaataatgtacgcaccatgcgcggattgcagtttgcagaagacgccagcccgatggcgcacccgatccgcccggatatggtcattgagatgaacaacttctacaccctgaccgtttacgagaagggcgcggaagtgattcgcatgatccacaccctgcttggcgaagaaaacttccagaaagggatgcagctttatttcgagcgtcatgatggtagtgcagcgacctgtgacgactttgtgcaggcgatggaagatgcgtcgaatgtcgatctctcccatttccgccgttggtacagccagtccggtacaccgattgtgaccgtcaaagacgactacaatccggaaaccgagcagtacaccctgaccatcagccagcgcacgccagccacgccggatcaggcagaaaaacagccgctgcatattccgtttgccatcgaactgtatgataacgaaggcaaagtgatcccgttgcagaaaggcggtcatccggtgaattccgtgctgaacgtcactcaggcggaacagacctttgtctttgataatgtctacttccagccggtgcctgcgctgctgtgcgaattctctgcgccagtgaaactggaatataagtggagcgatcagcaactgaccttcctgatgcgtcatgcgcgtaatgatttctcccgctgggatgcggcgcaaagtttgctggcaacctacatcaagctgaacgtcgcgcgtcatcagcaaggtcagccgctgtctctgccggtgcatgtggctgatgctttccgcgcggtactgcttgatgagaagattgatccagcgctggcggcagaaatcctgacgctgccttctgtcaatgaaatggctgaattgttcgatatcatcgacccgattgctattgccgaagtacgcgaagcactcactcgtactctggcgactgaactggcggatgagctactggctatttacaacgcgaattaccagagcgagtaccgtgttgagcatgaagatattgcaaaacgcactctgcgtaatgcctgcctgcgtttcctcgcttttggtgaaacgcatctggctgatgtgctggtgagcaagcagttccacgaagcaaacaatatgactgatgcgctggcggcgctttctgcggcggttgccgcacagctgccttgccgtgacgcgctgatgcaggagtacgacgacaagtggcatcagaacggtctggtgatggataaatggtttatcctgcaagccaccagcccggcggcgaatgtgctggagacggtgcgcggcctgttgcagcatcgctcatttaccatgagcaacccgaaccgtattcgttcgttgattggcgcgtttgcgggcagcaatccggcagcgttccatgccgaagatggcagcggttacctgttcctggtggaaatgcttaccgacctcaacagccgtaacccgcaggtggcttcacgtctgattgaaccgctgattcgcctgaaacgttacgatgccaaacgtcaggagaaaatgcgcgcggcgctggaacagttgaaagggctggaaaatctctctggcgatctgtacgagaagataactaaagcactggct

SEQ ID NO: 57 - DNA, 1605 bp, Escherichia coli
atgcgtatttccttgaaaaagtcagggatgctgaagcttggtctcagcctggtggctatgaccgtcgcagcaagtgttcaggctaaaactctggtttattgctcagaaggatctccggaagggtttaacccgcagctgtttacctccggcaccacctatgacgcctcttccgtcccgctttataaccgtctggttgaatttaaaatcggcaccaccgaagtgatcccgggcctcgctgaaaagtgggaagtcagcgaagacggtaaaacctataccttccatctgcgtaaaggtgtgaagtggcacgacaataaagaattcaaaccgacgcgtgaactgaacgccgatgatgtggtgttctcgttcgatcgtcagaaaaacgcgcaaaacccgtaccataaagtttctggcggcagctacgaatacttcgaaggcatgggcttgccagagctgatcagtgaagtgaaaaaggtggacgacaacaccgttcagtttgtgctgactcgcccggaagcgccgttcctcgctgacctggcaatggacttcgcctctattctgtcaaaagaatatgctgatgcgatgatgaaagccggtacaccggaaaaactggacctcaacccaatcggaaccggtccgttccagttacagcagtatcaaaaagattcccgtatccgctacaaagcgtttgatggctactggggcaccaaaccgcagatcgatacgctggttttctctattacccctgacgcttccgtgcgttacgcgaaattgcagaagaatgaatgccaggtgatgccgtacccgaacccggcagatatcgctcgcatgaagcaggataaatccatcaatctgatggaaatgccggggctgaacgtcggttatctctcgtataacgtgcagaaaaaaccactcgatgacgtgaaagttcgccaggctctgacctacgcggtgaacaaagacgcgatcatcaaagcggtttatcagggcgcgggcgtatcagcgaaaaacctgatcccgccaaccatgtggggctataacgacgacgttcaggactacacctacgatcctgaaaaagcgaaagccttgctgaaagaagcgggtctggaaaaaggtttctccatcgacctgtgggcgatgccggtacaacgtccgtataacccgaacgctcgccgcatggcggagatgattcaggcagactgggcgaaagtcggcgtgcaggccaaaattgtcacctacgaatggggtgagtacctcaagcgtgcgaaagatggcgagcaccagacggtaatgatgggctggactggcgataacggggatccggataacttcttcgccaccctgttcagctgcgccgcctctgaacaaggctccaactactcaaaatggtgctacaaaccgtttgaagatctgattcaaccggcgcgtgctaccgacgaccacaataaacgcgttgaactgtacaaacaagcgcaggtggtgatgcacgatcaggctccggcactgatcatcgctcactccaccgtgtttgaaccggtacgtaaagaagttaaaggctatgtggttgatccattaggcaaacatcacttcgaaaacgtctctatcgaa

SEQ ID NO: 58 - DNA, 1017 bp, Escherichia coli
atgttgcagtttattctccgacgtttgggactcgtcatccccacgtttatcggtattacccttctcacatttgcctttgtccacatgatcccgggcgatccggtgatgatcatggcgggcgaacgtgggatctccccagagcgtcacgcgcagctgctggctgaactcggcttagataaaccgatgtggcagcagtatctccattacatttggggcgttatgcatggcgatctaggcatttcaatgaaaagccgcatcccggtttgggaagagttcgtgccgcgcttccaggccacgctggaacttggcgtctgcgcgatgatttttgctacggcagtcggtattccggtcggcgtgctggctgcggttaaacgcggttccattttcgatcacacagcggttggcctggcgctgacaggttattcaatgcctatcttctggtggggcatgatgctgatcatgctggtttcggtgcactggaacctgacgcccgtctccggtcgcgtgagcgatatggtgttcctcgatgactccaatccgttaaccggttttatgctaatcgacaccgccatctggggtgaagacggcaactttatcgatgccgtcgcccatatgatcttgcctgccattgtgctgggtactattccgctggcggtcattgtgcgtatgacacgctcctcgatgctggaagtgctgggcgaggattacatccgcaccgcgcgcgccaaagggctaacccgcatgcgggtgattatcgtccatgcgctgcgtaacgcgatgctgccggtggtgaccgttatcggcctgcaggtgggaacattgctggcgggggcgattctgaccgaaaccatcttctcgtggcccggtctgggacgctggttgattgacgcactgcaacgccgcgactatccggtagtgcagggcggcgtattgctggtggcgacgatgattatcctcgtcaacttgctggtcgatctgctgtacggcgtggtgaacccgcgtattcgtcataagaag

SEQ ID NO: 59 - DNA, 900 bp, Escherichia coli
atgtcacaggttactgaaaataaagtgattagcgcaccggtgccgatgaccccgttacaggagttctggcactattttaaacgcaacaaaggcgcggtcgtcgggctggtttacgtcgtcatcgtgctgttcatcgcgatctttgccaactggattgcaccctataacccggcggaacagttccgcgatgcactgctcgccccgccagcctggcaggaaggcggcagcatggcgcacttgctgggcaccgatgacgtaggccgtgatgtgctgtcgcgcctgatgtacggtgcgcgcctgtcgctgctggttggctgtctggtagttgtgttatcgctgattatgggcgttattctcggcctgatcgccggttactttggcggcctggtcgataacatcattatgcgcgtggtcgatatcatgctggcgctgccaagtctgctgctggcgctggtgctggtggcaattttcggcccgtcgattggtaacgccgcgctggcactgaccttcgttgccttgccgcactatgtgcgcttaacccgcgccgccgtgctggtggaagttaaccgcgattacgtcaccgcgtctcgcgtggcgggtgccggggcgatgcgtcagatgtttattaacatcttcccgaactgccttgcgccgctgattgttcaggcgtcgctcggtttctctaacgccattctcgatatggctgctcttggtttcctcggcatgggggcacagccgccaacgcctgagtggggcaccatgctctccgacgtgttgcagttcgcgcaaagcgcctggtgggtcgtgaccttcccgggtctggcgatcctgctgacggtgctggcatttaacctgatgggtgacggtctgcgtgacgcgctcgatcccaaactgaagcag

SEQ ID NO: 60 - DNA, 981 bp, Escherichia coli
atggcgttattaaatgtagataaattatcggtgcatttcggcgacgaaagcgcgccgttccgcgccgtagaccgcatcagctacagcgtaaaacagggcgaagtggtcgggattgtgggtgagtccggctccggtaagtcggtcagttcactggcgattatggggctgattgattatccgggccgcgtaatggcagaaaaactggagtttaacggccaggatttgcagcgtatctcagaaaaagagcgccgcaacctggtgggtgccgaagtggcgatgatcttccaggacccgatgaccagccttaacccgtgctacaccgtgggtttccagattatggaagcgattaaggtgcatcagggcggcaacaaaagtacccgccgtcagcgagcgattgacctgctgaatcaggtcggtattcccgatccggcatcgcgtctggatgtttacccgcatcagctttccggcggcatgagccagcgcgtgatgatcgccatggcgattgcctgtcggccaaaactgctgattgccgatgaaccgaccaccgcgctggacgtgaccattcaggcgcaaatcatcgaactactgctggagctacagcagaaagagaacatggcgctggtgttaattacccatgacctggcgctggtggcggaagcggcacataaaatcatcgtgatgtatgcaggccaggtggtggaaaccggtgatgcgcacgccatcttccatgcgccgcgtcacccgtatactcaggcattgctgcgtgcgctgccagaatttgctcaggacaaagaacgtctggcgtcgttgccaggtgtcgttcccggcaagtacgaccgcccgaacggctgcctgcttaacccgcgctgcccctatgccactgacagatgtcgcgctgaagaaccggcgctgaatatgctcgctgacgggcgtcagtccaaatgccattacccacttgatgatgccgggaggccgacacta

SEQ ID NO: 61 - DNA, 1002 bp, Escherichia coli
atgagtacgcaagaggccaccctgcaacaaccgctgttgcaggctatcgacctgaaaaaacattatccggtgaagaaaggcatgttcgcgccggaacgtctggttaaagcgctggatggcgtttcgtttaaccttgaacgtggcaaaacgctggcagtagtgggcgaatctggctgcggtaaatcgaccctcggtcggttgctgacgatgattgaaatgcccaccggtggcgagctgtattaccaggggcaggatctgcttaagcacgatccgcaggcgcagaagctgcgtcggcagaaaatccagatcgtcttccagaacccttacggttcgctgaatccgcgtaaaaaagtcgggcaaattcttgaagagccgctgctgatcaacaccagcttaagcaaagaacagcgtcgggaaaaagccctgtcgatgatggcgaaagtcggcctgaaaaccgagcactatgaccgctatccgcatatgttctccggcggtcagcgtcagcgtatcgccatcgcccgtggtctgatgctcgacccggatgtggtgattgccgatgaaccggtttccgcgctggatgtttcagtgcgcgcgcaggtgctgaatctgatgatggatttgcagcaggagttggggctgtcttatgtctttatctcccacgacctgtcggtggtggagcacattgctgatgaagtgatggtgatgtacctgggccgctgcgtggagaagggaacgaaagaccaaatcttcaataacccgcgccatccgtacactcaggcgctactttccgcgacgccgcgcctgaacccggacgatcgccgcgagcgcatcaagctcagcggtgaactaccaagcccactgaatccaccgccgggttgcgccttcaacgcccgctgtcgtcggcgcttcggcccctgcacccagttgcagccgcagctaaaagactacggcggtcaactggtagcttgttttgctgttgatcaggatgaaaatccgcagcgt

SEQ ID NO: 62 - DNA, 56 bp, Artificial Sequence
  Description of Artificial Sequence: Synthetic DNA
ctaaccctgtgacctgcaatactgttttgcgggtgagtgtaggctggagctgcttc

SEQ ID NO: 63 - DNA, 56 bp, Artificial Sequence
  Description of Artificial Sequence: Synthetic DNA
gaaactgccggaaggcgattaaacgccatccggcagcatatgaatatcctccttag

SEQ ID NO: 64 - DNA, 56 bp, Artificial Sequence
  Description of Artificial Sequence: Synthetic DNA
ttacgcaacaggaatagactgaacaccagactctatgtgtaggctggagctgcttc

SEQ ID NO: 65 - DNA, 56 bp, Artificial Sequence
  Description of Artificial Sequence: Synthetic DNA
agaaaacaggggtaaattccccgaatggcggcgctacatatgaatatcctccttag

SEQ ID NO: 66 - DNA, 56 bp, Artificial Sequence
  Description of Artificial Sequence: Synthetic DNA
atggagtttagtgtaaaaagcggtagcccggagaaagtgtaggctggagctgcttc

SEQ ID NO: 67 - DNA, 56 bp, Artificial Sequence
  Description of Artificial Sequence: Synthetic DNA
ttactcttcgccgttaaacccagcgcggtttaacagcatatgaatatcctccttag

SEQ ID NO: 68 - DNA, 56 bp, Artificial Sequence
  Description of Artificial Sequence: Synthetic DNA
atgacagaagcgatgaagattaccctctctacccaagtgtaggctggagctgcttc

SEQ ID NO: 69 - DNA, 56 bp, Artificial Sequence
  Description of Artificial Sequence: Synthetic DNA
ttacgccgttaacagattagctatcgtgcgcacacccatatgaatatcctccttag

SEQ ID NO: 70 - DNA, 56 bp, Artificial Sequence
  Description of Artificial Sequence: Synthetic DNA
gcatccccacctcataacgttgacccgaccgggcaagtgtaggctggagctgcttc

SEQ ID NO: 71 - DNA, 56 bp, Artificial Sequence
  Description of Artificial Sequence: Synthetic DNA
ctgtacggcattttgctatgcttgtcgccactgttgcatatgaatatcctccttag

SEQ ID NO: 72 - DNA, 21 bp, Artificial Sequence
  Description of Artificial Sequence: Synthetic DNA
gtgtctgaactgtctcaatta

SEQ ID NO: 73 - DNA, 21 bp, Artificial Sequence
  Description of Artificial Sequence: Synthetic DNA
cggaatttctttcagcagttc

SEQ ID NO: 74 - DNA, 21 bp, Artificial Sequence
  Description of Artificial Sequence: Synthetic DNA
atgactcaacagccacaagcc

SEQ ID NO: 75 - DNA, 21 bp, Artificial Sequence
  Description of Artificial Sequence: Synthetic DNA
tgctttagttatcttctcgta

SEQ ID NO: 76 - DNA, 21 bp, Artificial Sequence
  Description of Artificial Sequence: Synthetic DNA
agtgcctgcatcgtcgtgggc

SEQ ID NO: 77 - DNA, 21 bp, Artificial Sequence
  Description of Artificial Sequence: Synthetic DNA
ggcgccttttgctttaccaga

SEQ ID NO: 78 - DNA, 21 bp, Artificial Sequence
  Description of Artificial Sequence: Synthetic DNA
gacgcgcgctggggagaaaaa

SEQ ID NO: 79 - DNA, 21 bp, Artificial Sequence
  Description of Artificial Sequence: Synthetic DNA
cgtagcgcccgcagaccactg

SEQ ID NO: 80 - DNA, 21 bp, Artificial Sequence
  Description of Artificial Sequence: Synthetic DNA
atgcgtatttccttgaaaaag

SEQ ID NO: 81 - DNA, 21 bp, Artificial Sequence
  Description of Artificial Sequence: Synthetic DNA
ttattcgatagagacgttttc

SEQ ID NO: 82 - DNA, 29 bp, Artificial Sequence
  Description of Artificial Sequence: Synthetic DNA
tacactcgagattaaagaggagaaattaa

SEQ ID NO: 83 - DNA, 30 bp, Artificial Sequence; identical to SEQ ID NO: 20
  Description of Artificial Sequence: Synthetic DNA

SEQ ID NO: 84 - DNA, 24 bp, Artificial Sequence
  Description of Artificial Sequence: Synthetic DNA
caagaattctcatgtttgacagct

SEQ ID NO: 85 - DNA, 28 bp, Artificial Sequence
  Description of Artificial Sequence: Synthetic DNA
taactcgagattccctttttacgtgaac

(19) United States
(12) Patent Application Publication
(10) Pub. No.: US 2008/0213827 A1
(43) Pub. Date: Sep. 4, 2008

(54) Process For Producing Dipeptides or Dipeptide Derivatives

(76) Inventors: Shin-ichi Hashimoto, Yamaguchi (JP); Hajime Ikeda, Yamaguchi (JP); Kazuhiko Tabata, Tokyo (JP)

Correspondence Address: FITZPATRICK CELLA HARPER & SCINTO, 30 ROCKEFELLER PLAZA, NEW YORK, NY, 10112, US

(21) Appl. No.: 11/630,397

(22) PCT Filed: Jun. 24, 2005
(86) PCT No.: PCT/JP2005/011638

§ 371 Date: Dec. 22, 2006

(30) Foreign Application Priority Data
Jun. 25, 2004 (JP) 2004-189008

(51) Int. Cl. C12P 21/04; C07K 7/00

(52) U.S. Cl. 435/68.1; 530/340

(57) ABSTRACT

The present invention provides a process for producing a dipeptide or a dipeptide derivative by using a protein having the activity to form the dipeptide or dipeptide derivative from one or more kinds of amino acids or amino acid derivatives, or a culture of cells having the ability to produce the protein or a treated matter of the culture as a enzyme source, which comprise;

allowing the enzyme source, one or more kinds of amino acids or amino acid derivatives and ATP to be present in an aqueous medium;
allowing the dipeptide or dipeptide derivative to form and accumulate in the medium; and
recovering the dipeptide or dipeptide derivative from the medium.

### TECHNICAL FIELD

The present invention relates to a process for producing a dipeptide or a dipeptide derivative, by using a protein having the activity to form the dipeptide or the dipeptide derivative, a culture of cells comprising a DNA encoding the protein or a treated matter of the culture, from one or more kinds of amino acids or amino acid derivatives.

### BACKGROUND OF THE INVENTION

Chemical synthesis methods (liquid phase method and solid phase method), enzymatic synthesis methods and biological synthesis methods utilizing recombinant DNA techniques are known as the methods for large-scale peptide synthesis. Currently, the enzymatic synthesis methods and biological synthesis methods are employed for the synthesis of long-chain peptides longer than 50 residues, and the chemical synthesis methods and enzymatic synthesis methods are mainly employed for the synthesis of dipeptides.

In the synthesis of dipeptides by the chemical synthesis methods, operations such as introduction and removal of protective groups for functional groups are necessary, and racemates are also formed. The chemical synthesis methods are thus considered to be disadvantageous in respect of cost and efficiency. They are unfavorable also from the viewpoint of environmental hygiene because of the use of large amounts of organic solvents and the like.

As to the synthesis of dipeptides by the enzymatic methods, the following methods are known: a method utilizing reverse reaction of protease (non-patent publication No. 1); methods utilizing thermostable aminoacyl t-RNA synthetase (Patent publication No. 1 to 4); and methods utilizing non-ribosomal peptide synthetase (hereinafter referred to as NRPS) (non-patent publication No. 2, 3 and patent publication 5, 6).

However, the method utilizing reverse reaction of protease requires introduction and removal of protective groups for functional groups of amino acids used as substrates, which causes difficulties in raising the efficiency of peptide-forming reaction and in preventing peptidolytic reaction. The methods utilizing thermostable aminoacyl t-RNA synthetase have the defects that the expression of the enzyme and the prevention of side reactions forming by-products other than the desired products are difficult. The methods utilizing NRPS are inefficient in that the expression of the enzyme by recombinant DNA techniques is difficult because of its large enzyme molecule size and that the supply of coenzyme 4′-phosphopantetheine is necessary.

On the other hand, there exist a group of peptide synthetases that have enzyme molecular weight lower than that of NRPS and do not require coenzyme 4′-phosphopantetheine; for example, γ-glutamylcysteine synthetase, glutathione synthetase, D-alanine-D-alanine (D-Ala-D-Ala) ligase, and poly-γ-glutamate synthetase. Most of these enzymes utilize D-amino acids as substrates or catalyze peptide bond formation at the γ-carboxyl group. Because of such properties, they can not be used for the synthesis of dipeptides by peptide bond formation at the α-carboxyl group of L-amino acid.

The only known example of an enzyme capable of dipeptide synthesis by the activity to form a peptide bond at the α-carboxyl group of L-amino acid is bacilysin (dipeptide antibiotic derived from a microorganism belonging to the genus Bacillus) synthetase. Bacilysin synthetase is known to have the activity to synthesize bacilysin [L-alanyl-L-anticapsin (L-Ala-L-anticapsin)] and L-alanyl-L-alanine (L-Ala-L-Ala), but there is no information about its activity to synthesize other peptides (non-patent publication No. 4 and 5).

As for the bacilysin biosynthetase genes in Bacillus subtilis 168 whose whole genome information has been clarified (non-patent publication No. 6), it is known that the productivity of bacilysin is increased by amplification of bacilysin operons containing ORFs ywfA-F (patent publication No. 7). However, it is not known whether an ORF encoding a protein having the activity to ligate two or more amino acids by peptide bond is contained in these ORFs, and if contained, which ORF encodes the protein.

Certain microorganisms are known to form a compound having the cyclic dipeptide (diketopiperazine) structure wherein two amino acids are cyclically bound (non-patent publication No. 7; 8 and 9). With regard to the biosynthesis of diketopiperazine, it is reported that the cyclo-(L-4-nitrotryptophyl-L-phenylalanine) structure is synthesized by NRPS in the Thaxtomin biosynthesis process of Streptomyces acidiscabies (non-patent publication No. 10) and that cyclo(phenylalanyl-proline) is formed from phenylalanine and proline by the action of a part of the modules of NRPS of bacteria belonging to the genus Bacillus (non-patent publication No. 11).

It is also reported that a protein bearing no similarity to NRPS (albC gene product) is responsible for the synthesis of the cyclo(L-phenylalanyl-L-leucine) structure in Streptomyces noursei ATCC 11455 known as a strain producing the antibiotic albonoursin and that albonoursin was detected when cyclo dipeptide oxidase was made to act on the culture broth of Escherichia coli and Streptomyces lividans into which the albC gene was introduced (non-patent publication No. 12). However, there is no report that the albC gene product forms a linear dipeptide.

As described above, there is no information that enzymes included in the bacylicin synthetase group have the activity to form various dipeptide derivatives from amino acids or amino acids derivatives.

Patent publication No. 1:

Japanese Published Unexamined Patent-Application No. 146539/83

Patent publication No. 2:

Japanese Published Unexamined Patent Application No. 209991/83

Patent publication No. 3:

Japanese Published Unexamined Patent Application No. 209992/83

Patent publication No. 4:

Japanese Published Unexamined Patent Application No. 106298/84

Patent publication No. 5:

U.S. Pat. No. 5,795,738

Patent publication No. 6:

U.S. Pat. No. 5,652,116

Patent publication No. 7:

WO00/03009 pamphlet

Non-patent publication No. 1:

J. Biol. Chem., 119, 707-720 (1937)

Non-patent publication No. 2:

Chem. Biol., 7, 373-384 (2000)

Non-patent publication No. 3:

FEBS Lett., 498, 42-45 (2001)

Non-patent publication No. 4:

J. Ind. Microbiol., 2, 201-208 (1987)

Non-patent publication No. 5:

Enzyme. Microbial. Technol., 29, 400-406 (2001)

Non-patent publication No. 6:

Nature, 390, 249-256 (1997)

Non-patent publication No. 7:

J. Nat. Prod., 59, 293-296 (1996)

Non-patent publication No. 8:

Tetrahedron, 28, 2999 (1972)

Non-patent publication No. 9:

J. Appl. Microbiol., 86, 29-53 (1999)

Non-patent publication No. 10:

Mol. Microbiol., 38, 794-804 (2000)

Non-patent publication No. 11:

J. Biol. Chem., 273, 22773-22781 (1998)

Non-patent publication No. 12:

Chemistry & Biol., 9, 1355-1364 (2002)

### DISCLOSURE OF THE INVENTION

### Problems to be Solved by the Invention

An object of the present invention is to provide a process for producing dipeptide derivatives from amino acids or amino acid derivatives.

### Means for Solving the Problems

The present invention relates to the following (1) to (26).
(1) A process for producing a dipeptide or a dipeptide derivative (PI) (hereinafter referred to as dipeptide or dipeptide derivative PI), which comprises:
   allowing the protein shown in any of the following [1] to [7], one or more kinds of amino acids or amino acid derivatives and ATP to be present in an aqueous medium;
   allowing the dipeptide or dipeptide derivative PI to form and accumulate in the aqueous medium; and
   recovering the dipeptide or dipeptide derivative PI from the aqueous medium;
   [1] a protein having the amino acid sequence shown in any of SEQ ID NOS: 1 to 8;
   [2] a protein consisting of an amino acid sequence wherein one or more amino acid residues are deleted, substituted or added in the amino acid sequence shown in any of SEQ ID NOS: 1 to 8 and having the activity to form the dipeptide or dipeptide derivative PI from one or more kinds of amino acids or amino acid derivatives;
   [3] a protein consisting of an amino acid sequence which has 65% or more homology to the amino acid sequence shown in any of SEQ ID NOS: 1 to 8 and having the activity to form the dipeptide or dipeptide derivative PI from one or more kinds of amino acids or amino acid derivatives;
   [4] a protein consisting of an amino acid sequence which has 80% or more homology to the amino acid sequence shown in SEQ ID NO: 17 and having the activity to form the dipeptide or dipeptide derivative PI from one or more kinds of amino acids or amino acid derivatives;
   [5] a protein having the amino acid sequence shown in SEQ ID NO: 37 or 38;
   [6] a protein consisting of an amino acid sequence wherein one or more amino acid residues are deleted, substituted or added in the amino acid sequence shown in SEQ ID NO: 37 or 38 and having the activity to form the dipeptide or dipeptide derivative PI from one or more kinds of amino acids or amino acid derivatives;
   [7] a protein consisting of an amino acid sequence which has 65% or more homology to the amino acid sequence shown in SEQ ID. NO: 37 or 38 and having the activity to form the dipeptide or dipeptide derivative PI from one or more kinds of amino acids or amino acid derivatives;
   provided that a compound wherein the amino acids selected from the following Amino acid group A are the same or different, and are bound by peptide bond is excluded from the dipeptide or dipeptide derivative PI:
   Amino acid group A; L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutanoic acid, L-azaserine, L-theanine, L-4-hydroxyproline, L-3-hydroxyproline, L-ornithine, L-citrulline, L-6-diazo-5-oxo-norleucine, glycine and β-alanine.
(2) A process for producing a dipeptide or a dipeptide derivative (hereinafter referred to as dipeptide or dipeptide derivative PII), which comprises:
   allowing the protein shown in any of following [1] to [7], one or more kinds of amino acids or amino acid derivatives and ATP to be present in an aqueous medium;
   allowing dipeptide or dipeptide derivative PI to form and accumulate in the aqueous medium;
   subjecting the dipeptide or dipeptide derivative PI, as such or after recovery, to modification to form the dipeptide or dipeptide derivative PII; and
   recovering the dipeptide or dipeptide derivative PII;
   [1] a protein having the amino acid sequence shown in any of SEQ ID NOS: 1 to 8;
   [2] a protein consisting of an amino acid sequence wherein one or more amino acid residues are deleted, substituted or added in the amino acid sequence shown in any of SEQ ID NOS: 1 to 8 and having the activity to form the dipeptide or dipeptide derivative PI from one or more kinds of amino acids or amino acid derivatives;
   [3] a protein consisting of an amino acid sequence which has 65% or more homology to the amino acid sequence shown in any of SEQ ID NOS: 1 to 8 and having the activity to form the dipeptide or dipeptide derivative PI from one or more kinds of amino acids or amino acid derivatives;
   [4] a protein consisting of an amino acid sequence which has 80% or more homology to the amino acid sequence shown in SEQ ID NO: 17 and having the activity to form the dipeptide or dipeptide derivative PI from one or more kinds of amino acids or amino acid derivatives;
   [5] a protein having the amino acid sequence shown in SEQ ID NO: 37 or 38;
   [6] a protein consisting of an amino acid sequence wherein one or more amino acid residues are deleted, substituted or added in the amino acid sequence shown in SEQ ID NO: 37 or 38 and having the activity to form the dipeptide or dipeptide derivative PI from one or more kinds of amino acids or amino acid derivatives;
   [7] a protein consisting of an amino acid sequence which has 65% or more homology to the amino acid sequence shown in SEQ ID NO: 37 or 38 and having the activity to form the dipeptide or dipeptide derivative PI from one or more kinds of amino acids or amino acid derivatives;
   provided that a compound wherein the amino acids selected from the Amino acid group A described in the above are the same or different, and are bound by peptide bond is excluded from the dipeptide or dipeptide derivative PII.
(3) A process for producing a dipeptide or a dipeptide derivative PI, which comprises:
   allowing an enzyme source and one or more kinds of amino acids or amino acid derivatives to be present in an aqueous medium, said enzyme source being a culture or a treated matter of the culture of cells having the DNA selected from the following [1] to [5];
   [1] DNA having the nucleotide sequence shown in any of SEQ ID NOS: 9 to 16 and 36;
   [2] DNA which hybridizes with DNA having a nucleotide sequence complementary to the nucleotide sequence shown in any of SEQ ID NOS: 9 to 16 and 36 under stringent conditions and which encodes a protein having the activity to form the dipeptide or dipeptide derivative PI from one or more kinds of amino acids or amino acid derivatives;
   [3] DNA which hybridizes with DNA having a nucleotide sequence complementary to the nucleotide sequence shown in SEQ ID NO: 18 under stringent conditions and which encodes a protein having the activity to form the dipeptide or dipeptide derivative PI from one or more kinds of amino acids or amino acid derivatives;
   [4] DNA having the nucleotide sequence shown in SEQ ID NO: 39 or 40;
   [5] DNA which hybridizes with DNA having a nucleotide sequence complementary to the nucleotide sequence shown in SEQ ID NO: 39 or 40 under stringent conditions and which encodes a protein having the activity to form the dipeptide or dipeptide derivative PI from one or more kinds of amino acids or amino acid derivatives;
   provided that a compound wherein the amino acids selected from the Amino acid group A described in the above are the same or different, and are bound by peptide bond is excluded from the dipeptide or dipeptide derivative PI.
(4) A process for producing a dipeptide or a dipeptide derivative PII, which comprises:
   allowing an enzyme source and one or more kinds of amino acids or amino acid derivatives to be present in an aqueous medium, said enzyme source being a culture or a treated matter of the culture of cells having the DNA selected from the following [1] to [5]; allowing dipeptide or dipeptide derivative PI to form and accumulate in the aqueous medium;
   subjecting the dipeptide or dipeptide derivative PI, as such or after recovery, to modification to form the dipeptide or dipeptide derivative PII; and
   recovering the dipeptide or dipeptide derivative PII;
   [1] DNA having the nucleotide sequence shown in any of SEQ ID NOS: 9 to 16 and 36;
   [2] DNA which hybridizes with DNA having a nucleotide sequence complementary to the nucleotide sequence shown in any of SEQ ID NOS: 9 to 16 and 36 under stringent conditions and which encodes a protein having the activity to form the dipeptide or dipeptide derivative PI from one or more kinds of amino acids or amino acid derivatives;
   [3] DNA which hybridizes with DNA having a nucleotide sequence complementary to the nucleotide sequence shown in SEQ ID NO: 18 under stringent conditions and which encodes a protein having the activity to form the dipeptide or dipeptide derivative PI from one or more kinds of amino acids or amino acid derivatives;
   [4] DNA having the nucleotide sequence shown in SEQ ID NO: 39 or 40;
   [5] DNA which hybridizes with DNA having a nucleotide sequence complementary to the nucleotide sequence shown in SEQ ID NO: 39 or 40 under stringent conditions and which encodes a protein having the activity to form the dipeptide or dipeptide derivative PI from one or more kinds of amino acids or amino acid derivatives.
(5) The process according to any of the above (1) to (4), wherein the amino acids or amino acid derivatives are amino acids or amino acid derivatives represented by formula (I):

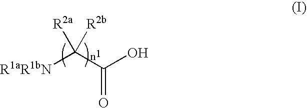

(wherein n¹ represents an integer of 1 to 3; R^{1a} and R^{1b}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or either R^{1a} or
R^{1b} may form a substituted or unsubstituted heterocyclic group together with the adjacent nitrogen atom, the carbon atom adjacent to the nitrogen atom and either R^{2a} or R^{2b} on the carbon atom; and
R^{2a} and R^{2b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{2a} or R^{2b} on the carbon atom adjacent to R^{1a}R^{1b}N may form a substituted or unsubstituted heterocyclic group together with the adjacent carbon atom, the nitrogen atom adjacent to the carbon atom and either R^{1a} or R^{1b}, and when n¹ is 2 or 3, two or three R² as and two or three R^{2b}s may be the same or different, respectively), or formula (II):

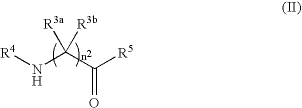

[wherein n² has the same significance as the above n¹;
R^{3a} and R^{3b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{3a} or R^{3b} on the carbon atom adjacent to R⁴HN may form a substituted or unsubstituted heterocyclic group together with the adjacent carbon atom, the nitrogen atom adjacent to the carbon atom and R⁴, and when n² is 2 or 3, two or three R³ as and two or three R^{3b}s may be the same or different, respectively;
R⁴ represents a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or R⁴ may form a substituted or unsubstituted heterocyclic group together with the adjacent nitrogen atom, the carbon atom adjacent to the nitrogen atom and either R^{3a} or R^{3b} on the carbon atom; and
R⁵ represents amino, hydroxy, substituted or unsubstituted lower alkoxy, mono(substituted or unsubstituted lower alkyl)amino, di(substituted or unsubstituted lower alkyl)amino, or an alicyclic heterocyclic group],
provided that when all the amino acids or amino acid derivatives are amino acids or amino acid derivatives represented by formula (I), at least one of R^{1a} and R^{1b} is a hydrogen atom, and when all the amino acids or amino acid derivatives are amino acids or amino acid derivatives represented by formula (II), R⁵ is hydroxy.
(6) The process according to any of the above (1) to (4), wherein the amino acids or amino acid derivatives are amino acids or amino acid derivatives represented by formula (III):

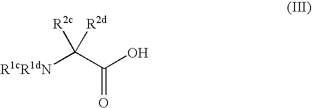

(wherein R^{1c} and R^{1d}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl; and
R^{2c} and R^{2d}, which may be the same or different, each represent a hydrogen atom or substituted or unsubstituted lower alkyl),
or formula (IV):

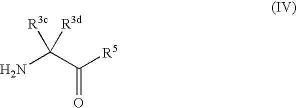

(wherein R^{3c} and R^{3d}, which may be the same or different, each represent a hydrogen atom, substituted or unsubstituted lower alkyl, substituted or unsubstituted aralkyl, or substituted or unsubstituted aryl; and
R⁵ has the same significance as defined above), provided that when all the amino acids or amino acid derivatives are amino acids or amino acid derivatives represented by formula (III), at least one of R^{1c} and R^{1d} is a hydrogen atom, and when all the amino acids or amino acid derivatives are amino acids or amino acid derivatives represented by formula (IV), R⁵ is hydroxy.
(7) The process according to any of the above (1) to (4), wherein the amino acids or amino acid derivatives are amino acids or amino acid derivatives represented by formula (V):

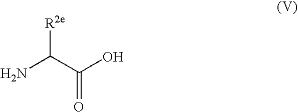

(wherein R^{2e} represents substituted or unsubstituted methyl),
or formula (VI):

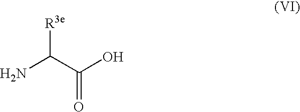

(wherein R^{3e} represents substituted or unsubstituted lower alkyl, substituted or unsubstituted aralkyl, or substituted or unsubstituted aryl).
(8) The process according to any of the above (1) to (4), wherein the amino acids or amino acid derivatives are amino acids selected from the group consisting of L-amino acids, glycine and β-alanine, or derivatives thereof.
(9) The process according to the above (8), wherein the L-amino acid is an L-amino acid selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine, L-citrulline and L-6-diazo-5-oxo-norleucine.
(10) The process according to any of the above (1) to (5), wherein the dipeptide or dipeptide derivative PI is a dipeptide or a dipeptide derivative represented by formula (VIIa):

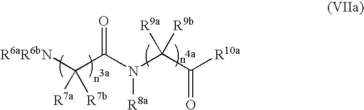

[wherein n^{3a} and n^{4a} each have the same significance as the above n¹;
R^{6a} and R^{6b}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or either R^{6a} or R^{6b} may form a substituted or unsubstituted heterocyclic group together with the adjacent nitrogen atom, the carbon atom adjacent to the nitrogen atom and either R^{7a} or R^{7b} on the carbon atom;
R^{7a} and R^{7b}, which may be the same or different, each represents a hydrogen atom, substituted or unsubstituted lower alkyl, substituted or unsubstituted aralkyl, substituted or unsubstituted aryl, or substituted or unsubstituted heterocyclic alkyl, or either R^{7a} or R^{7b} on the carbon atom adjacent to R^{6a}R^{6b}N may form a substituted or unsubstituted heterocyclic group together with the adjacent carbon atom, the nitrogen atom adjacent to the carbon atom and either R^{6a} or R^{6b}, and when n^{3a} is 2 or 3, two or three R^{7a}s and two or three R^{7b}s may be the same or different, respectively;
R^{8a} represents a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or R^{8a} may form a substituted or unsubstituted heterocyclic group together with the adjacent nitrogen atom, the carbon atom adjacent to the nitrogen atom and bound to R^{9a} and R^{9b}, and either R^{9a} or R^{9b} on the carbon atom;
R^{9a} and R^{9b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{9a} or R^{9b} on the carbon atom adjacent to —R^{8a}N— may form a substituted or unsubstituted heterocyclic group together with the adjacent carbon atom, the nitrogen atom adjacent to the carbon atom and R^{8a}, and when n^{4a} is 2 or 3, two or three R⁹ as and two or three R^{9b}s may be the same or different, respectively; and
R^{10a} represents amino, hydroxy, substituted or unsubstituted lower alkoxy, mono(substituted or unsubstituted lower alkyl)amino, di(substituted or unsubstituted lower alkyl)amino, or an alicyclic heterocyclic group].
(11) The process according to any of the above (1) to (5), wherein the dipeptide or dipeptide derivative PII is a dipeptide or a dipeptide derivative represented by formula (VIIb):

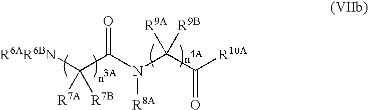

(wherein n^{3A} and n^{4A} each have the same significance as the above n¹;
R^{6A} and R^{6B}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or either R^{6A} or R^{6B} may form a substituted or unsubstituted heterocyclic group together with the adjacent nitrogen atom, the carbon atom adjacent to the nitrogen atom and either R^{7A} or R^{7B} on the carbon atom;
R^{7A} and R^{7B}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{7A} or R^{7B} on the carbon atom adjacent to R^{6A}R^{6B}N may form a substituted or unsubstituted heterocyclic group together with the adjacent carbon atom, the nitrogen atom adjacent to the carbon atom and either R^{6A} or R^{6B}, and when n^{3A} is 2 or 3, two or three R^{7A}s and two or three R^{7b}s may be the same or different, respectively;
R^{8A} represents a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or R^{8A} may form a substituted or unsubstituted heterocyclic group together with the adjacent nitrogen atom, the carbon atom adjacent to the nitrogen atom and bound to R^{9A} and R^{9B}, and either R^{9A} or R^{9B} on the carbon atom;
R^{9A} and R^{9B}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{9A} or R^{9B} on the carbon atom adjacent to —R^{8A}N— may form a substituted or unsubstituted heterocyclic group together with the adjacent carbon atom, the nitrogen atom adjacent to the carbon atom and R^{8A}, and when n^{4A} is 2 or 3, two or three R^{9A}s and two or three R^{9B}s may be the same or different, respectively; and
R^{10A} has the same significance as the above R^{10a}).
(12) The process according to any of the above (1) to (6), wherein the dipeptide or dipeptide derivative PI is a dipeptide or a dipeptide derivative represented by formula (VIIIa):

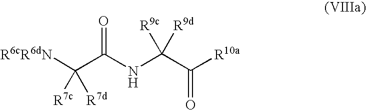

(wherein R^{6c} and R^{6d}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl;
R^{7c} and R^{7d}, which may be the same or different, each represent a hydrogen atom or substituted or unsubstituted lower alkyl;
R^{9c} and R^{9d}, which may be the same or different, each represent a hydrogen atom, substituted or unsubstituted lower alkyl, substituted or unsubstituted aralkyl, or substituted or unsubstituted aryl; and
R^{10a} has the same significance as defined above).
(13) The process according to any of the above (1) to (6), wherein the dipeptide or dipeptide derivative PII is a dipeptide or a dipeptide derivative represented by formula (VIIIb):

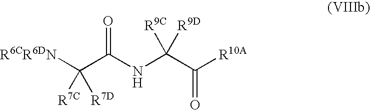

(wherein R^{6C}, R^{6D}, R^{7C}, R^{7D}, R^{9C} and R^{9D} have the same significances as the above R^{6c}, R^{6d}, R^{7c}, R^{7d}, R^{9c} and R^{9d}, respectively; and
R^{10A} has the same significance as defined above).
(14) The process according to any of the above (1) to (7),
   wherein the dipeptide or dipeptide derivative PI is a dipeptide or a dipeptide derivative represented by formula (IXa):

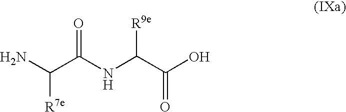

(wherein R^{7e} represents substituted or unsubstituted methyl; and
R^{9e} represents substituted or unsubstituted lower alkyl, substituted or unsubstituted aralkyl, or substituted or unsubstituted aryl).
(15) The process according to any of the above (1) to (7), wherein the dipeptide or dipeptide derivative PII is a dipeptide or a dipeptide derivative represented by formula (IXb):

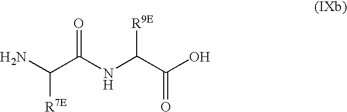

(wherein R^{7E} and R^{9E} have the same significances as the above R^{7e} and R^{9e}, respectively).
(16) The process according to any of the above (1) to (8), wherein the dipeptide or dipeptide derivative PI or dipeptide or dipeptide derivative PII is a dipeptide or a dipeptide derivative in which the same or different amino acids or amino acid derivatives selected from the group consisting of L-amino acids, glycine, 1-alanine and their derivatives are bound by peptide bond.
(17) The process according to the above (16), wherein the L-amino acid is an L-amino acid selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine, L-citrulline and L-6-diazo-5-oxo-norleucine.
(18) The process according to any of the above (3) to (17), wherein the cells are cells of a microorganism.
(19) The process according to the above (18), wherein the microorganism is a procaryote.
(20) The process according to the above (19), wherein the procaryote is a microorganism in which the activities of one or more kinds of peptidases and one or more kinds of proteins having peptide-permeating/transporting activity (hereinafter referred to also as peptide-permeating/transporting proteins) are reduced or lost.
(21) The process according to the above (20), wherein the procaryote is a microorganism in which the activities of three or more kinds of peptidases are reduced or lost.
(22) The process according to the above (20) or (21), wherein the peptidase is a protein having the amino acid sequence shown in any of SEQ ID NOS: 43 to 46, or a protein having an amino acid sequence which has 80% or more homology to the amino acid sequence shown in any of SEQ ID NOS: 43 to 46 and having peptidase activity.
(23) The process according to the above (20) or (22), wherein the peptide-permeating/transporting protein is a protein having the amino acid sequence shown in any of SEQ ID NOS: 47 to 51, or a protein having an amino acid sequence which has 80% or more homology to the amino acid sequence shown in any of SEQ ID NOS: 47 to 51 and having peptide-permeating/transporting activity.
(24) The process according to any of the above (19) to (23), wherein the procaryote is a microorganism belonging to the genus Escherichia, Bacillus or Corynebacterium.
(25) The process according to the above (24), wherein the microorganism belonging to the genus Escherichia, Bacillus or Corynebacterium is Escherichia coli, Corynebacterium glutamicum, Corynebacterium ammoniagenes, Corynebacterium lactofermentum, Corynebacterium flavum, Corynebacterium efficiens, Bacillus subtilis or Bacillus megaterium.
(26) The process according to any of the above (3) to (25), wherein the treated matter of the culture is a treated matter which is selected from the group consisting of concentrated culture, dried culture, cells obtained by centrifuging the culture, dried culture, cells obtained by centrifuging the culture, dried cells, freeze-dried cells, surfactant-treated cells, ultrasonicated cells, mechanically, disrupted cells, solvent-treated cells, enzymatic-treated cells, protein fractionation of cells, immobilized cells, and an enzyme preparation obtained by extracting the cells, and which has the activity to form dipeptide or dipeptide derivative from one or more kinds of amino acids or amino acid derivatives, and which has the activity to form dipeptide or dipeptide derivative from one or more kinds of amino acids or amino acid derivatives.

### EFFECT OF THE INVENTION

In accordance with the present invention, dipeptide derivatives can be efficiently produced from one or more kinds of amino acids or amino acid derivatives.

### BRIEF DESCRIPTION OF THE DRAWINGS

FIG. 1 shows the steps for constructing plasmid pPE43.

FIG. 2 shows the steps for constructing plasmid pQE60ywfE.

FIG. 3 shows the steps for constructing pAL-nou and pAL-alb, which are plasmid vectors for the expression of proteins having the activity to synthesize a linear dipeptide.

FIG. 4 shows the steps for constructing ywfE gene expression-enhanced vector pPE56.

### EXPLANATION OF SYMBOLS

ywfE: ywfE gene derived from Bacillus subtilis 168
Ptrp: Tryptophan promoter gene
PT5: T5 promoter
Amp^{r}: Ampicillin resistance gene
lacI^{q}: Lactose repressor gene
albC: albC gene or albC-analogous gene

### BEST MODES FOR CARRYING OUT THE INVENTION

### 1. Proteins Used in the Present Invention

Examples of the proteins used in the present invention include:

[1] a protein having the amino acid sequence shown in any of SEQ ID NOS: 1 to 8;
[2] a protein consisting of an amino acid sequence wherein one or more amino acid residues are deleted, substituted or added in the amino acid sequence shown in any of SEQ ID NOS: 1 to 8 and having the activity to form a dipeptide or a dipeptide derivative from one or more kinds of amino acids or amino acid derivatives;
[3] a protein consisting of an amino acid sequence which has 65% or more homology to the amino acid sequence shown in any of SEQ ID NOS: 1 to 8 and having the activity to form a dipeptide or a dipeptide derivative from one or more kinds of amino acids or amino acid derivatives;
[4] a protein consisting of an amino acid sequence which has 80% or more homology to the amino acid sequence shown in SEQ ID NO: 17 and having the activity to form a dipeptide or a dipeptide derivative from one or more kinds of amino acids or amino acid derivatives;
[5] a protein having the amino acid sequence shown in SEQ ID NO: 37 or 38;
[6] a protein consisting of an amino acid sequence wherein one or more amino acid residues are deleted, substituted or added in the amino acid sequence shown in SEQ ID NO.: 37 or 38 and having the activity to form a dipeptide or a dipeptide derivative from one or more kinds of amino acids or amino acid derivatives;
[7] a protein consisting of an amino acid sequence which has 65% or more homology to the amino acid sequence shown in SEQ ID NO: 37 or 38 and having the activity to form a dipeptide or a dipeptide derivative from one or more kinds of amino acids or amino acid derivatives.

The above protein consisting of an amino acid sequence wherein one or more amino acid residues are deleted, substituted or added and having the activity to form dipeptide represented by formula (I) can be obtained by introducing a site-directed mutation into DNA encoding a protein consisting of the amino acid sequence shown in any of SEQ ID NOS: 1 to 8, 37 and 38, by site-directed mutagenesis described in Molecular Cloning, A Laboratory Manual, Third Edition, Cold Spring Harbor Laboratory Press (2001) (hereinafter referred to as Molecular Cloning, Third Edition); Current Protocols in Molecular Biology, John Wiley & Sons (1987-1997) (hereinafter referred to as Current Protocols in Molecular Biology); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc.

The number of amino acid residues which are deleted, substituted or added is not specifically limited, but is within the range where deletion, substitution or addition is possible by known methods such as the above site-directed mutagenesis. The suitable number is 1 to dozens, preferably 1 to 20, more preferably 1 to 10, further preferably 1 to 5.

The expression “one or more amino acid residues are deleted, substituted or added in the amino acid sequence shown in any of SEQ ID NOS: 1 to 8, 37 and 38” means that the amino acid sequence may contain deletion, substitution or addition of a single or plural amino acid residues at an arbitrary position therein.

Amino acid residues that may be substituted are, for example, those which are not conserved in all of the amino acid sequences shown in SEQ ID NOS: 1 to 8, 37 and 38 when the sequences are compared using known alignment software. An example of known alignment software is alignment analysis software contained in gene analysis software Genetyx (Software Development Co., Ltd.). As analysis parameters for the analysis software, default values can be used.

Deletion or addition of amino acid residues may be contained, for example, in the N-terminal region or the C-terminal region of the amino acid sequence shown in any of SEQ ID NOS: 1 to 8, 37 and 38.

Deletion, substitution and addition may be simultaneously contained in one sequence, and amino acids to be substituted or added may be either natural or not. Examples of the natural amino acids are L-alanine, L-asparagine, L-aspartic acid, L-arginine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine and L-cysteine.

The following are examples of the amino acids capable of mutual substitution. The amino acids in the same group can be mutually substituted.
Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2-aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

In order that the protein using in the present invention may have the activity to form dipeptides or dipeptide derivatives, it is desirable that the homology of its amino acid sequence to the amino acid sequence shown in any of SEQ ID NOS: 1 to 8, 37 and 38, preferably SEQ ID NO: 1 or 37, is 65% or more, preferably 75% or more, more preferably 85% or more, further preferably 90% or more, particularly preferably 95% or more, and most preferably 98% or more.

The homology among amino acid sequences and nucleotide sequences can be determined by using algorithm BLAST by Karlin and Altschul [Proc. Natl. Acad. Sci. USA, 90, 5873 (1993)] and FASTA [Methods Enzymol., 183, 63 (1990)]. On the basis of the algorithm BLAST, programs such as BLASTN and BLASTX have been developed [J. Mol. Biol., 215, 403 (1990)]. When a nucleotide sequence is analyzed by BLASTN on the basis of BLAST, the parameters, for instance, are as follows: score=100 and wordlength=12. When an amino acid sequence is analyzed by BLASTX on the basis of BLAST, the parameters, for instance, are as follows: score=50 and wordlength=3. When BLAST and Gapped BLAST programs are used, default parameters of each program are used. The specific techniques for these analyses are known (http://www.ncbi.nlm.nih.gov.).

A protein consisting of an amino acid sequence which has 65% or more, preferably 75% or more, more preferably 85% or more, further preferably 90% or more, particularly preferably 95% or more, most preferably 98% or more homology to the amino acid sequence shown in any of SEQ ID NOS: 1 to 8, 37 and 38, preferably SEQ ID NO: 1 or 37, and having the activity to form dipeptides or dipeptide derivatives is also included in the proteins used in the present invention. The homology among amino acid sequences can be determined by using BLAST or FASTA as described above.

The amino acid sequence shown in SEQ ID NO: 17 is a region conserved among the proteins having the amino acid sequences shown in SEQ ID NOS: 1 to 7 and is also a region corresponding to the consensus sequence of proteins having Ala-Ala ligase activity derived from various microorganisms.

A protein consisting of an amino acid sequence which has 80% or more, preferably 90% or more, more preferably 95% or more homology to the amino acid sequence shown in SEQ ID NO: 17 and having the activity to form dipeptides of dipeptide derivatives is also included in the proteins used in the present invention.

In order that the protein having an amino acid sequence which has 80% or more, preferably 90% or more, further preferably 95% or more homology to the amino acid sequence shown in SEQ ID NO: 17 may have the activity to form dipeptides or dipeptide derivatives, it is desirable that the homology of its amino acid sequence to the amino acid sequence shown in any of SEQ ID NOS: 1 to 8 is at least 80% or more, usually 90% or more, and particularly 95% or more.

The homology among amino acid sequences can be determined by using BLAST or FASTA as described above.

It is possible to confirm that the protein used in the present invention is a protein having the activity to form dipeptides or dipeptide derivatives, for example, in the following manner. That is, a transformant expressing the protein used in the present invention is prepared by recombinant DNA techniques, the protein used in the present invention is produced using the transformant, and then the protein, one or more kinds of L-amino acids and amino acid derivatives and ATP are allowed to be present in an aqueous medium, followed by HPLC analysis or the like to know whether a dipeptide or a dipeptide derivative is formed and accumulated in the aqueous medium.

### 2. DNAs Used in the Present Invention

Examples of the DNAs used in the present invention include DNAs:

[1] DNA having the nucleotide sequence shown in any of SEQ ID NOS: 9 to 16 and 36;
[2] DNA which hybridizes with DNA having a nucleotide sequence complementary to the nucleotide sequence shown in any of SEQ ID NOS: 9 to 16 and 36 under stringent conditions and which encodes a protein having the activity to form a dipeptide or a dipeptide derivative from one or more kinds of amino acids or amino acid derivatives;
[3] DNA which hybridizes with DNA having a nucleotide sequence complementary to the nucleotide sequence in SEQ ID NO: 18 and encoding a protein having the activity to form a dipeptide or a dipeptide derivative from one or more kinds of amino acids or amino acid derivatives;
[4] DNA having the nucleotide sequence shown in SEQ ID NO: 39 or 40; and
[5] DNA which hybridizes with DNA having a nucleotide sequence complementary to the nucleotide sequence shown in SEQ ID NO: 39 or 40 under stringent conditions and which encodes a protein having the activity to form a dipeptide or a dipeptide derivative from one or more kinds of amino acids or amino acid derivatives.

The above DNA capable of hybridization under stringent conditions refers to DNA which is obtained by colony hybridization, plaque hybridization, Southern blot hybridization, or the like using a part or the whole of the DNA having a nucleotide sequence complementary to the nucleotide sequence shown in any of SEQ ID NOS: 9 to 16, 18, 36, 39 and 40 as a probe. A specific example of such DNA is DNA which can be identified by performing hybridization at 65° C. in the presence of 0.7 to 1.0 mol/l, preferably 0.9 mol/l sodium chloride using a filter with colony- or plaque-derived DNA immobilized thereon, and then washing the filter at 65° C. with a 0.1 to 2-fold conc., preferably 0.1-fold conc. SSC solution (1-fold conc. SSC solution: 150 mmol/l sodium chloride and 15 mmol/l sodium citrate). Hybridization can be carried out according to the methods described in Molecular Cloning, Third Edition; Current Protocols in Molecular Biology; DNA Cloning 1: Core Techniques, A Practical Approach, Second Edition, Oxford University (1995), etc. Specifically, the hybridizable DNA includes DNA having at least 75% or more homology, preferably 85% or more homology, further preferably 90% or more homology, particularly preferably 95% or more homology to the nucleotide sequence shown in any of SEQ ID NOS: 9 to 16, 18, 36, 39 and 40 as calculated by use of BLAST or FASTA described above based on the above parameters.

It is possible to confirm that the DNA which hybridizes with DNA having the nucleotide sequence shown in any of SEQ ID NOS: 9 to 16, 18, 36, 39 and 40 under stringent conditions is DNA encoding a protein having the activity to form dipeptides or dipeptide derivatives, for example, by producing a protein encoded by the DNA by recombinant DNA techniques and measuring the activity of the protein as described above.

### 4. Preparation of DNA Used in the Present Invention

The DNA used in the present invention can be obtained by Southern hybridization of a chromosomal DNA library from a microorganism belonging to the genus Bacillus or Streptomyces using a probe designed based on the nucleotide sequence shown in SEQ ID NOS: 9 to 16, 36, 39 or 40, or by PCR [PCR Protocols, Academic Press (1990)] using primer DNAs designed based on the nucleotide sequence shown in SEQ ID NOS: 9 to 16, 36, 39 or 40, as a template, the chromosomal DNA of a microorganism belonging to the genus Bacillus.

The DNA used in the present invention can also be obtained by conducting a search through various gene sequence databases for a sequence having 75% or more homology, preferably 85% or more homology, more preferably 90% or more homology, further preferably 95% or more homology, particularly preferably 98% or more homology to the nucleotide sequence of DNA encoding the amino acid sequence shown in any of SEQ ID NOS: 1 to 8, 17, 37 and 38, and obtaining the desired DNA, based on the nucleotide sequence obtained by the search, from a chromosomal DNA or cDNA library of an organism having the nucleotide sequence according to the above-described method.

The obtained DNA, as such or after cleavage with appropriate restriction enzymes, is inserted into a vector by a conventional method, and the obtained recombinant DNA is introduced into a host cell. Then, the nucleotide sequence of the DNA can be determined by a conventional sequencing method such as the dideoxy method [Proc. Natl. Acad. Sci., USA, 74, 5463 (1977)] or by using a nucleotide sequencer such as 373A DNA Sequencer (Perkin-Elmer Corp.).

In cases where the obtained DNA is found to be a partial DNA by the analysis of nucleotide sequence, the full length DNA can be obtained by Southern hybridization of a chromosomal DNA library using the partial DNA as a probe.

It is also possible to prepare the desired DNA by chemical synthesis using a DNA synthesizer (e.g., Model 8905, PerSeptive Biosystems) based on the determined nucleotide sequence of the DNA.

Examples of the DNAs that can be obtained by the above-described method are DNAs having the nucleotide sequences shown in SEQ ID NOS: 9 to 16, 36, 39 and 40.

Examples of the vectors for inserting the DNA of the present invention and the DNA used in the production process of the present invention include pBluescriptII KS(+) (Stratagene), pDIRECT [Nucleic Acids Res., 18, 6069 (1990)], pCR-Script Amp SK(+) (Stratagene), pT7 Blue (Novagen, Inc.), pCR II (Invitrogen Corp.) and pCR-TRAP (Genhunter Corp.).

As the host cell, microorganisms belonging to the genus Escherichia, etc. can be used. Examples of the microorganisms belonging to the genus Escherichia include Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli MP347, Escherichia coli NM522 and Escherichia coli ME8415.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

Examples of the microorganisms carrying the DNAs used in the production process of the present invention obtained by the above method are Escherichia coli NM522/pPE43, which is a microorganism carrying a recombinant DNA comprising DNA having the nucleotide sequence shown in SEQ ID NO: 1.

### 4. Preparation of Cells Used in the Present Invention

(1) The cells used in the present invention include:
i) bacteria belonging to the genus Bacillus which carry the DNA of the above 3 on the chromosomal DNA, preferably, bacteria belonging to the genus Bacillus which have bacilysin-synthesizing activity, more preferably, bacteria belonging to a species selected from the group consisting of Bacillus subtilis, Bacillus amyloliquefaciens, Bacillus coagulans, Bacillus licheniformis, Bacillus megaterium and Bacillus pumilus, further preferably, bacteria selected from the group consisting of the strains Bacillus subtilis ATCC 15245, Bacillus subtilis ATCC 6633, Bacillus subtilis IAM 1213, Bacillus subtilis IAM 1107, Bacillus subtilis IAM 1214, Bacillus subtilis ATCC 9466, Bacillus subtilis IAM 1033, Bacillus subtilis ATCC 21555, Bacillus amyloliquefaciens IFO 3022 and Bacillus pumilus NRRL B-12025;
ii)-bacteria belonging to the genus Streptomyces which carry the DNA of the above 3 on the chromosomal DNA, preferably, bacteria belonging to the genus Streptomyces which have albonoursin-synthesizing activity, more preferably, bacteria belonging to a species selected from Streptomyces albulus and Streptomyces noursei, further preferably, Streptomyces albulus IFO 14147, Streptomyces noursei ATCC 11455 or IFO 15452; and
iii)transformants that can be prepared by introducing DNA obtained by the method of the above 3 into host cells using the methods described in Molecular Cloning, Third Edition, Current Protocols in Molecular Biology, etc., for example, according to the method described below.

On the basis of the DNA used in the present invention, a DNA fragment of an appropriate length comprising a region encoding the protein used in the present invention is prepared according to need. Cells with enhanced productivity of the protein can be obtained by replacing a nucleotide in the nucleotide sequence of the region encoding the protein so as to make a codon most suitable for the expression in a host cell.

The DNA fragment is inserted downstream of a promoter in an appropriate expression vector to prepare a recombinant DNA.

A transformant producing the protein of the present invention can be obtained by introducing the recombinant DNA into a host cell suited for the expression vector.

As the host cell, any cells that are capable of expressing the desired gene can be used. Suitable cells include cells of microorganisms such as procaryotes and yeast, animal cells, insect cells, plant cells, etc., preferably microorganisms, more preferably procaryotes, further preferably bacteria.

The expression vectors that can be employed are those capable of autonomous replication or integration into the chromosome in the above host cells and comprising a promoter at a position appropriate for the transcription of the DNA of the present invention or the DNA used in the production process of the present invention.

When a procaryote such as a bacterium is used as the host cell, it is preferred that the recombinant DNA comprising the DNA used in the present invention is a recombinant DNA which is capable of autonomous replication in the procaryote and which comprises a promoter, a ribosome binding sequence, the DNA of the present invention or the DNA used in the production process of the present invention, and a transcription termination sequence. The recombinant DNA may further comprise a gene regulating the promoter.

Examples of suitable expression vectors are pBTrp2, pBTac1 and pBTac2 (products of Boehringer Mannheim GmbH), pHelixl (Roche Diagnostics Corp.), pKK233-2 (Amersham Pharmacia Biotech), pSE280 (Invitrogen Corp.), pGEMEX-1 (Promega Corp.), pQE-8 (Qiagen, Inc.), pET-3 (Novagen, Inc.), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Shuzo Co., Ltd.), pUC118 (Takara Shuzo Co., Ltd.), pPA1 (Japanese Published Unexamined Patent Application No. 233798/88), pWH1520 (MoBiTec), pCS299P (WO00/63388), pVLT31 [Gene, 123, 17 (1993)] and pIJ702 (Genetic Manipulation of Streptomyces: a Laboratory Manual: John Innes Foundation).

As the promoter, any promoters capable of functioning in host cells such as Escherichia coli can be used. For example, promoters derived from Escherichia coli or phage, such as trp promoter (Pₜᵣₚ), lac promoter (P_{lac}), P_{L} promoter, P_{R} promoter and P_{SE} promoter, S_{POL} promoter, SPO2 promoter and penP promoter can be used. Artificially designed and modified promoters such as a promoter in which two Pₜᵣₚs are combined in tandem, tac promoter, lacT7 promoter and letI promoter, etc. can also be used.

Also useful are xylA promoter for the expression in microorganisms belonging to the genus Bacillus [Appl. Microbiol. Biotechnol., 35, 594-599 (1991)], P54-6 promoter for the expression in microorganisms belonging to the genus Corynebacterium [Appl. Microbiol. Biotechnol., 53, 674-679 (2000)], tac promoter for the expression in microorganisms belonging to the genus Pseudomonas [Gene, 123, 17-24 (1993)] and xylA promoter for the expression in microorganisms belonging to the genus Streptomyces (Genetic Manipulation of Streptomyces: a Laboratory Manual: John Innes Foundation).

It is preferred to use a plasmid in which the distance between the Shine-Dalgarno sequence (ribosome binding sequence) and the initiation codon is adjusted to an appropriate length (e.g., 6 to 18 nucleotides).

In the recombinant DNA wherein the DNA used in the present invention is ligated to an expression vector, the transcription termination sequence is not essential, but it is preferred to place the transcription termination sequence immediately downstream of the structural gene.

Examples of such recombinant DNAs are pPE43.

Examples of suitable procaryotes include microorganisms belonging to the genera Escherichia, Serratia, Bacillus, Brevibacterium, Corynebacterium, Microbacterium, Pseudomonas, Agrobacterium, Alicyclobacillus, Anabaena, Anacystis, Arthrobacter, Azotobacter, Chromatium, Erwinia, Methylobacterium, Phormidium, Rhodobacter, Rhodopseudomonas, Rhodospirillum, Scenedesmus, Streptomyces, Synechoccus and Zymomonas. Specific examples are Escherichia coli XL1-Blue, Escherichia coli XL2-Blue, Escherichia coli DH1, Escherichia coli DH5α, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli MP347, Escherichia coli NM522, Bacillus subtilis ATCC 33712, Bacillus megaterium, Bacillus sp. FERM BP-6030, Bacillus amyloliquefaciens, Bacillus coagulans, Bacillus licheniformis, Bacillus pumilus, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 14297, Corynebacterium acetoacidophilum ATCC 13870, Microbacterium ammoniaphilum ATCC 15354, Serratia ficaria, Serratia fonticola, Serratia liquefaciens, Serratia marcescens, Pseudomonas sp. D-0110, Agrobacterium radiobacter, Agrobacterium rhizogenes, Agrobacterium rubi, Anabaena cylindrica, Anabaena doliolum, Anabaena flos-aquae, Arthrobacter aurescens, Arthrobacter citreus, Arthrobacter globformis, Arthrobacter hydrocarboglutamicus, Arthrobacter mysorens, Arthrobacter nicotianae, Arthrobacter paraffineus, Arthrobacter protophormiae, Arthrobacter roseoparaffinus, Arthrobacter sulfureus, Arthrobacter ureafaciens, Chromatium buderi, Chromatium tepidum, Chromatium vinosum, Chromatium warmingii, Chromatium fluviatile, Erwinia uredovora, Erwinia carotovora, Erwinia ananas, Etwinia herbicola, Erwinia punctata, Erwinia terreus, Methylobacterium rhodesianum, Methylobacterium extorquens, Phormidium sp. ATCC 29409, Rhodobacter capsulatus, Rhodobacter sphaeroides, Rhodopseudomonas blastica, Rhodopseudomonas marina, Rhodopseudomonas palustris, Rhodospirillum rubrum, Rhodospirillum salexigens, Rhodospirillum salinarum, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus and Zymomonas mobilis. Preferred procaryotes include bacteria belonging to the genera Escherichia, Serratia, Bacillus, Brevibacterium, Corynebacterium, Pseudomonas and Streptomyces, for example, the above-mentioned species belonging to the genera Escherichia, Serratia, Bacillus, Brevibacterium, Corynebacterium, Pseudomonas and Streptomyces. More preferred bacteria include Escherichia coli, Corynebacterium glutamicum, Corynebacterium ammoniagenes, Corynebacterium lactofermentum, Corynebacterium flavum, Corynebacterium efficiens, Bacillus subtilis, Bacillus megaterium, Serratia marcescens, Pseudomonas putida, Pseudomonas aeruginosa, Streptomyces coelicolor and Streptomyces lividans, among which Escherichia coli is particularly preferred.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

When a yeast strain is used as the host cell, YEp13 (ATCC 37115), YEp24 (ATCC 37051), YCp50 (ATCC 37419), pHS19, pHS15, etc. can be used as the expression vector.

As the promoter, any promoters capable of functioning in yeast strains can be used. Suitable promoters include PHO5 promoter, PGK promoter, GAP promoter, ADH promoter, gal 1 promoter, gal 10 promoter, heat shock polypeptide promoter, MFα1 promoter and CUP 1 promoter.

Examples of suitable host cells are yeast strains belonging to the genera Saccharomyces, Schizosaccharomyces, Kluyveromyces, Trichosporon, Schwanniomyces, Pichia and Candida, specifically, Saccharomyces cerevisiae, Schizosaccharomyces pombe, Kluyveromyces lactis, Trichosporon pullulans, Schwanniomyces alluvius, Pichia pastoris and Candida utilis.

Introduction of the recombinant DNA can be carried out by any of the methods for introducing DNA into yeast, for example, electroporation [Methods Enzymol., 194, 182 (1990)], the spheroplast method [Proc. Natl. Acad. Sci. USA, 81, 4889 (1984)] and the lithium acetate method [J. Bacteriol., 153, 163 (1983)].

When an animal cell is used as the host cell, pcDNAI, pcDM8 (commercially available from Funakoshi Co., Ltd.), pAGE107 (Japanese Published Unexamined Patent Application No. 22979/91), pAS3-3 (Japanese Published Unexamined Patent Application No. 227075/90), pCDM8 [Nature, 329, 840 (1987)], pcDNAI/Amp (Invitrogen Corp.), pREP4 (Invitrogen Corp.), pAGE103 [J. Biochem., 101, 1307 (1987)], pAGE210, pAMo, pAMoA, etc. can be used as the expression vector.

As the promoter, any promoters capable of functioning in animal cells can be used. Suitable promoters include the promoter of IE (immediate early) gene of cytomegalovirus (CMV), SV40 early promoter, metallothionein promoter, the promoter of a retrovirus, heat shock promoter, SR α promoter, etc. The enhancer of IE gene of human CMV may be used in combination with the promoter.

Examples of suitable host cells are mouse myeloma cells, rat myeloma cells, mouse hybridomas, human-derived Namalwa cells and Namalwa KJM-1 cells, human embryonic kidney cells, human leukemia cells, African green monkey kidney cells, Chinese hamster-derived CHO cells, and HBT5637 (Japanese Published Unexamined Patent Application No. 299/88).

The mouse myeloma cells include SP2/0 and NSO; the rat myeloma cells include YB2/0; the human embryonic kidney cells include HEK293 (ATCC CRL-1573); the human leukemia cells include BALL-1; and the African green monkey kidney cells include COS-1 and COS-7.

Introduction of the recombinant DNA can be carried out by any of the methods for introducing DNA into animal cells, for example, electroporation [Cytotechnology, 3, 133 (1990)], the calcium phosphate method (Japanese Published Unexamined Patent Application No. 227075/90), lipofection [Proc. Natl. Acad. Sci. USA, 84, 7413 (1987)], and the method described in Virology, 52, 456 (1973).

When an insect cell is used as the host cell, the protein can be produced by using the methods described in Baculovirus Expression Vectors, A Laboratory Manual, W. H. Freeman and Company, New York (1992); Current Protocols in Molecular Biology; Molecular Biology, A Laboratory Manual; Bio/Technology, 6, 47 (1988), etc.

That is, the recombinant gene transfer vector and a baculovirus are cotransfected into insect cells to obtain a recombinant virus in the culture supernatant of the insect cells, and then insect cells are infected with the recombinant virus, whereby the protein can be produced.

The gene transfer vectors useful in this method include pVL1392, pVL1393 and pBlueBacIII (products of Invitrogen Corp.).

An example of the baculovirus is Autographa californica nuclear polyhedrosis virus, which is a virus infecting insects belonging to the family Barathra.

Examples of the insect cells are ovarian cells of Spodoptera frugiperda, ovarian cells of Trichoplusia ni, and cultured cells derived from silkworm ovary.

The ovarian cells of Spodoptera frugiperda include Sf9 and Sf21 (Baculovirus Expression Vectors, A Laboratory Manual); the ovarian cells of Trichoplusia ni include High 5 and BTI-TN-5B1-4 (Invitrogen Corp.); and the cultured cells derived from silkworm ovary include Bombyx mori N4.

Cotransfection of the above recombinant gene transfer vector and the above baculovirus into insect cells for the preparation of the recombinant virus can be carried out by the calcium phosphate method (Japanese Published Unexamined Patent Application No. 227075/90), lipofection [Proc. Natl. Acad. Sci. USA, 84, 7413 (1987)], etc.

When a plant cell is used as the host cell, Ti plasmid, tobacco mosaic virus vector, etc. can be used as the expression vector.

As the promoter, any promoters capable of functioning in plant cells can be used. Suitable promoters include 35S promoter of cauliflower mosaic virus (CaMV), rice actin 1 promoter, etc.

Examples of suitable host cells are cells of plants such as tobacco, potato, tomato, carrot, soybean, rape, alfalfa, rice, wheat and barley.

Introduction of the recombinant vector can be carried out by any of the methods for introducing DNA into plant cells, for example, the method using Agrobacterium (Japanese Published Unexamined Patent Application Nos. 140885/84 and 70080/85, WO94/00977), electroporation (Japanese Published Unexamined Patent Application No. 251887/85) and the method using particle gun (gene gun) (Japanese Patent Nos. 2606856 and 2517813).

When the DNA is expressed in yeast, an animal cell, an insect cell or a plant cell, a cell producing a glycosylated protein can be obtained.

(2) The microorganisms preferrably used in the production process of the present invention include microorganisms prepared by the method of the above (1) in which the activities of one or more kinds of peptidases and one or more kinds of proteins having peptide-permeating/transporting activity (hereinafter referred to as peptide-permeating/transporting proteins) are reduced or lost, and those in which the activities of three or more kinds of peptidases are reduced or lost.

Such microorganisms can be obtained, for example, by any of the following methods: (a) methods of imparting, by the method of the above (1), the ability to produce the protein having the dipeptide forming activity or the ability to produce the protein having polyphosphate kinase activity to microorganisms in which the functions of one or more kinds of peptidases and one or more kinds of proteins having peptide-permeating/transporting activity are reduced or lost, or microorganisms in which the functions of three or more kinds of peptidases are reduced or lost; and (b) methods of reducing or causing loss of the functions of a) one or more kinds of peptidases and one or more kinds of peptide-permeating/transporting proteins or b) three or more kinds of peptidases of microorganisms having the ability to produce the protein having the dipeptide-forming activity or the ability to produce the protein having polyphosphate kinase activity which can be prepared by the method of the above (1).

The microorganisms in which the activities of one or more kinds of peptidases and one or more kinds of peptide-permeating/transporting proteins are reduced or lost include microorganisms in which the activities of one or more arbitrary kinds of peptidases and one or more arbitrary kinds of peptide-permeating/transporting proteins are reduced or lost provided that the microorganisms can normally grow, specifically, microorganisms in which the activities of preferably one to nine kinds, more preferably one to seven kinds, further preferably one to four kinds of peptidases and preferably one to five kinds, more preferably one to three kinds, further preferably one or two kinds, particularly preferably one kind of peptide-permeating/transporting protein are reduced or lost.

Examples of such microorganisms are microorganisms in which the activities of one or more kinds of peptidases and one or more kinds of peptide-permeating/transporting proteins are reduced or lost because the nucleotide sequences of one or more kinds of genes encoding peptidases (hereinafter referred to as peptidase genes) and one or more kinds of genes encoding peptide-permeating/transporting proteins (hereinafter referred to as peptide-permeating/transporting protein genes) among the peptidase genes and peptide-permeating/transporting protein genes existing on the genomic DNA of the microorganisms are entirely or partially deleted or said nucleotide sequences contain nucleotide substitutions or additions.

The expression “the activity of peptidase is reduced” means that the peptidolytic activity is reduced, or reduced to normally 80% or less, preferably 50% or less, more preferably 30% or less, further preferably 20% or less, particularly preferably 10% or less, most preferably 5% or less compared with peptidase encoded by the gene having none of the above deletions, substitutions and additions of nucleotides.

The peptidolytic activity of a microorganism can be measured by allowing a peptide as a substrate and microorganism cells to be present in an aqueous medium, thereby performing peptidolytic reaction, and then determining the amount of the remaining peptide by a known method, e.g., HPLC analysis.

The above peptidases may be any proteins having peptidolytic activity. Preferred are proteins having high dipeptide-hydrolyzing activity. More preferred are dipeptidases.

Examples of peptidases include: those existing in Escherichia coli such as PepA having the amino acid sequence shown in SEQ ID NO: 43, PepB having the amino acid sequence shown in SEQ ID NO: 44, PepD having the amino acid sequence shown in SEQ ID NO: 45, PepN having the amino acid sequence shown in SEQ ID NO: 46, PepP [GenBank accession No. (hereinafter abbreviated as Genbank) AAC75946], PepQ (GenBank AAC76850), PepE (GenBank AAC76991), PepT (GenBank AAC74211), Dcp (GenBank AAC74611) and IadA (GenBank AAC77284); those existing in Bacillus subtilis such as AmpS (GenBank AF012285), PepT (GenBank X99339), YbaC (GenBank Z99104), YcdD (GenBank Z99105), YjbG (GenBank Z99110), YkvY (GenBank Z99111), YqjE (GenBank Z99116) and YwaD (GenBank Z99123); and those existing in Corynebacterium glutamicum such as proteins having the amino acid sequences represented by BAB97732, BAB97858, BAB98080, BAB98880, BAB98892, BAB99013, BAB99598 and BAB99819 (registration Nos. of DNA Data Bank of Japan). Examples of dipeptidases include PepA, PepB, PepD and PepN having the amino acid sequences shown in SEQ ID NOS: 43 to 46, PepQ, PepE and IadA. Proteins having amino acid sequences which have 80% or more, preferably 90% or more, more preferably 95% or more homology to the amino acid sequence shown in any of SEQ ID NOS: 43 to 46 and having peptidase activity are also included in the proteins having high dipeptide-hydrolyzing activity. The homology among amino acid sequences and nucleotide sequences can be determined by using BLAST, FASTA or the like described above.

The expression “the activity of a peptide-permeating/transporting protein is reduced” means that the peptide-uptaking activity is reduced, or reduced to normally 80% or less, preferably 50% or less, more preferably 30% or less, further preferably 20% or less, particularly preferably 10% or less, most preferably 5% or less compared with a peptide-permeating/transporting protein encoded by the gene having none of the above deletions, substitutions and additions of nucleotides.

The peptide-uptaking activity of a microorganism can be measured by allowing a peptide as a substrate and microorganism cells to be present in an aqueous medium, thereby performing peptide-uptaking reaction, and then determining the amount of the remaining peptide by a known method, e.g., HPLC analysis.

The above peptide-permeating/transporting proteins may be any proteins involved in peptide uptake of microorganisms, for example, proteins encoded by genes forming an operon on chromosomal DNA which form a complex on cell membrane to express dipeptide-uptaking activity and those which have peptide-uptaking activity as individual proteins. Preferred are proteins having high peptide-uptaking activity.

Examples of the peptide-permeating/transporting proteins include: those existing in Escherichia coli such as DppA having the amino acid sequence shown in SEQ ID NO: 47, DppB having the amino acid sequence shown in SEQ ID NO: 48, DppC having the amino acid sequence shown in SEQ ID NO: 49, DppD having the amino acid sequence shown in SEQ ID NO: 50, DppF having the amino acid sequence shown in SEQ ID NO: 51, OppA (GenBank AAC76569), OppB (GenBank AAC76568), OppC (GenBank AAC76567), OppD (GenBank AAC76566), OppF (GenBank AAC76565), YddO (GenBank AAC74556), YddP (GenBank AAC74557), YddQ (GenBank AAC74558), YddR (GenBank AAC74559), YddS (GenBank AAC74560), YbiK (GenBank AAC73915), MppA (GenBank AAC74411), SapA (GenBank AAC74376), SapB (GenBank AAC74375), SapC (GenBank AAC74374), SapD (GenBank AAC74373) and SapF (GenBank AAC74372); those existing in Bacillus subtilis such as DppA (GenBank CAA40002), DppB (GenBank CAA40003), DppC (GenBank CAA40004), DppD (GenBank CAA40005), DppE (GenBank CAA40006), OppA (GenBank CAA39787), OppB (GenBank CAA39788), OppC (GenBank CAA39789), OppD (GenBank CAA39790), OppF (GenBank CAA39791), AppA (GenBank CAA62358), AppB (GenBank CAA62359), AppC (GenBank CAA62360), AppD (GenBank CAA62356), AppF (GenBank CAA62357), YclF (GenBank CAB12175) and YkfD (GenBank CAB13157); and those existing in Corynebacterium glutamicum such as proteins having the amino acid sequences represented by BAB99048, BAB99383, BAB99384, BAB99385, BAB99713, BAB99714, BAB99715, BAB99830, BAB99831 and BAB99832 (registration Nos. of DNA Data Bank of Japan). Examples of the proteins having high peptide-permeating/transporting activity include DppA, DppB, DppC, DppD and DppF having the amino acid sequences shown in SEQ ID NOS: 47 to 51, and proteins having amino acid sequences which have 80% or more, preferably 90% or more, more preferably 95% or more homology to the amino acid sequence shown in any of SEQ ID NOS: 47 to 51.

The homology among amino acid sequences can be determined by using programs such as BLAST and FASTA described above.

The microorganisms in which the activities of three or more kinds of peptidases are reduced or lost include microorganisms in which the activities of three or more arbitrary kinds of peptidases are reduced or lost provided that the microorganisms can normally grow, specifically, microorganisms in which the activities of preferably three to nine kinds, more preferably three to six kinds, further preferably three or four kinds of peptidases are reduced or lost.

Examples of peptidases include the above-described peptidases and dipeptidases existing in Escherichia coli, Bacillus subtilis and Corynebacterium glutamicum. Proteins consisting of amino acid sequences which have 80% or more, preferably 90% or more, more preferably 95% or more homology to the amino acid sequence shown in any of SEQ ID NOS: 43 to 46 and having peptidase activity are also included in the proteins having high dipeptide-hydrolyzing activity.

The homology among amino acid sequences can be determined by using programs such as BLAST and FASTA described above.

The microorganisms in which the activities of peptidases and peptide-permeating/transporting proteins are reduced or lost may be obtained by any method capable of preparing such microorganisms. For example, they can be obtained by introducing a deletion, substitution or addition of a nucleotide into peptidase genes and peptide-permeating/transporting protein genes on chromosomal DNAs of microorganisms as described below.

The methods for introducing a deletion, substitution or addition of a nucleotide into a gene on the chromosomal DNA of a microorganism include methods utilizing homologous recombination. An example of the methods utilizing general homologous recombination is a method using a plasmid for homologous recombination prepared by ligating a mutant gene having an introduced nucleotide deletion, substitution or addition to a plasmid DNA incapable of autonomous replication in a host cell into which the nucleotide deletion or the like is to be introduced and carrying a drug resistance gene.

The plasmid for homologous recombination is introduced into a host cell by an ordinary method, followed by selection of a transformant in which the plasmid for homologous recombination has been integrated into the chromosomal DNA by homologous recombination using the drug resistance as a marker. The obtained transformant is cultured using a medium which does not contain the drug for several hours to one day, and then spread on an agar medium containing the drug and on an agar medium without the drug. By selecting a strain which does not grow on the former medium but can grow on the latter medium, the strain in which second homologous recombination occurred on the chromosomal DNA can be obtained. Introduction of a nucleotide deletion, substitution or addition into a desired gene on the chromosomal DNA can be confirmed by determining the nucleotide sequence of a region of the chromosomal DNA containing the gene into which the deletion or the like has been introduced.

By use of the above method, a nucleotide deletion, substitution or addition can be introduced into desired genes on chromosomal DNAs of microorganisms such as those belonging to the genera Escherichia, Bacillus and Corynebacterium.

Further, a nucleotide deletion, substitution or addition can be efficiently introduced into plural genes by utilizing homologous recombination according to a method using a linear DNA.

Specifically, a linear DNA containing a gene into which a nucleotide deletion, substitution or addition is to be introduced is incorporated into a cell to cause homologous recombination between chromosomal DNA and the introduced linear DNA. This method is applicable to any microorganisms capable of efficiently incorporating a linear DNA. Preferred microorganisms are those belonging to the genera Escherichia and Bacillus. Escherichia coli is more preferred, and Escherichia coli expressing a group of recombinant proteins derived from λ phage (Red recombination system) is further preferred.

An example of Escherichia coli expressing λ Red recombination system is Escherichia coli JM101 carrying pKD46, which is a plasmid DNA comprising a λ Red recombination system gene (available from Escherichia coli Genetic Stock Center, Yale University, U.S.A.).

Examples of the DNAs useful for homologous recombination are as follows:

(a) linear DNA in which DNAs present on the outside of a region of chromosomal DNA to be subjected to introduction of a nucleotide deletion, substitution or addition are present at both termini of a drug resistance gene;
(b) linear DNA in which DNAs present on the outside of a region of chromosomal DNA to be subjected to introduction of a nucleotide deletion, substitution or addition are directly ligated to each other;
(c) linear DNA in which DNAs present on the outside of a region of chromosomal DNA to be subjected to introduction of a nucleotide deletion, substitution or addition are present at both termini of a drug resistance gene and a gene that can be used for negative selection; (d) linear DNA of the above (a) in which a nucleotide sequence recognized by yeast-derived Flp recombinase [Proc. Natl. Acad. Sci. USA., 82, 5875 (1985)] is additionally present between the drug resistance gene and the DNAs present on the outside of a region of chromosomal DNA.

As the drug resistance gene, any drug resistance genes that impart resistance to a drug to which the host microorganism shows sensitivity can be used. When Escherichia coli is used as the host microorganism, examples of the drug resistance genes are kanamycin resistance gene, chloramphenicol resistance gene, gentamicin resistance gene, spectinomycin resistance gene, tetracycline resistance gene and ampicillin resistance gene.

The “gene that can be used for negative selection” refers to a gene that is fatal to a host microorganism under certain culture conditions when the gene is expressed in the host microorganism. Examples of the genes are sacB gene derived from a microorganism belonging to the genus Bacillus [Appl. Environ. Microbiol., 59, 1361-1366 (1993)] and rpsL gene derived from a microorganism belonging to the genus Escherichia [Genomics, 72, 99-104 (2001)].

The DNAs having homology to the DNAs present on the outside of a region of chromosomal DNA to be subjected to introduction of a substitution or deletion in the above linear DNAs are located in the same direction as that on the chromosomal DNA, and their length is preferably about 10 bp to 100 bp, more preferably about 20 bp to 50 bp, and further preferably about 30 bp to 40 bp.

The nucleotide sequence recognized by yeast-derived Flp recombinase is not specifically limited so long as it is a nucleotide sequence recognized by the said protein and catalyzing homologous recombination. Preferred examples are DNA having the nucleotide sequence shown in SEQ ID NO: 52, and DNA having a nucleotide sequence wherein one to several nucleotides are deleted, substituted or added in the said DNA and having a nucleotide sequence recognized by yeast-derived Flp recombinase and catalyzing homologous recombination.

The expression “having homology” refers to having such a degree of homology that allows occurrence of homologous recombination between the subject region of chromosomal DNA and the above linear DNA, specifically, having 80% or more homology, preferably 90% or more homology, more preferably 95% or more homology, further preferably 100% homology.

The homology among nucleotide sequences can be determined by using programs such as BLAST and FASTA described above.

The above linear DNA fragments can be prepared by PCR. The desired linear DNA can also be obtained by constructing DNA containing the above linear DNA on plasmid and then carrying out treatment with restriction enzymes.

Examples of the methods for introducing a nucleotide deletion, substitution or addition into the chromosomal DNA of a microorganism include the following Methods 1 to 4.

### Method 1:

A method which comprises introducing the linear DNA of the above (a) or (d) into a host microorganism and selecting a transformant carrying the linear DNA inserted on its chromosomal DNA by homologous recombination using the drug resistance as a marker.

### Method 2:

A method which comprises introducing the linear DNA of the above (b) into the transformant obtained according to the above Method 1 and eliminating the drug resistance gene inserted on its chromosomal DNA by Method 1 to substitute or delete a region of the chromosomal DNA of the microorganism.

### Method 3:

A method which comprises:

[1] introducing the linear DNA of the above (c) into a host microorganism and selecting a transformant carrying the linear DNA inserted on its chromosomal DNA by homologous recombination using the drug resistance as a marker;
[2] synthesizing DNA by ligating DNAs having homology to the DNAs present on the outside of a region of chromosomal DNA to be subjected to introduction of a substitution or deletion in the same direction as that on the chromosomal DNA, and introducing the synthesized DNA into the transformant obtained in the above [1]; and
[3] culturing the transformant subjected to the operation of the above [2] under conditions such that the gene that can be used for negative selection is expressed, and selecting a strain capable of growing by the culturing as a strain in which the drug resistance gene and the gene that can be used for negative selection are eliminated from the chromosomal DNA.

### Method 4:

A method which comprises:

[1] introducing the linear DNA of the above (d) into a host microorganism and selecting a transformant carrying the linear DNA inserted on its chromosomal DNA by homologous recombination using the drug resistance as a marker; and
[2] introducing a Flp recombinase gene expression plasmid into the transformant obtained in the above [1], and after expression of the gene, obtaining a strain sensitive to the drug used in the above [1].

In the above methods, introduction of the linear DNA into a host microorganism can be carried out by any of the methods for introducing DNA into the microorganism, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

By using a linear DNA in which an arbitrary gene to be inserted to chromosomal DNA is incorporated in the center part of the linear DNA used in Method 2 or Method 3 [2], it is possible to eliminate the drug resistance gene and at the same time to insert an arbitrary gene to the chromosomal DNA.

The above Methods 2 to 4 are methods that leave no foreign genes such as a drug resistance gene and a gene usable for negative selection on the chromosomal DNA of the transformant to be finally obtained. Therefore, it is possible to readily produce a microorganism having nucleotide deletions, substitutions or additions in two or more different regions of the chromosomal DNA by repeating the operations of the Methods using the same drug resistance gene and the same gene usable for negative selection.

### 5. Preparation of the Protein Used in the Present Invention

The protein can be produced by culturing the transformant obtained by the method of the above 4 in a medium, allowing the protein used in the present invention to form and accumulate in the culture, and recovering the protein from the culture.

The host of the above transformant for producing the protein used in the present invention may be any microorganism such as procaryote or yeast, animal cell, insect cell, plant cell or the like, but is preferably a microorganism, more preferably a procaryote, further preferably a bacterium, particularly preferably a bacterium belonging to the genus Escherichia, and most preferably Escherichia coli.

Culturing of the above transformant in a medium can be carried out by conventional methods for culturing the host cell.

For the culturing of the transformant microorganism obtained by using a procaryote such as Escherichia coli or a eucaryote such as yeast as the host cell, any of natural media and synthetic media can be used insofar as it is a medium suitable for efficient culturing of the transformant which contains carbon sources, nitrogen sources, inorganic salts, etc. which can be assimilated by the host used.

As the carbon sources, any carbon sources that can be assimilated by the host can be used. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

As the nitrogen sources, ammonia, ammonium salts of organic or inorganic acids such as ammonium chloride, ammonium sulfate, ammonium acetate and ammonium phosphate, and other nitrogen-containing compounds can be used as well as peptone, meat extract, yeast extract, corn steep liquor, casein hydrolyzate, soybean cake, soybean cake hydrolyzate, and various fermented microbial cells and digested products thereof.

Examples of the inorganic salts include potassium dihydrogenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration. The culturing temperature is preferably 15 to 40° C., and the culturing period is usually 5 hours to 7 days. The pH is maintained at 3.0 to 9.0 during the culturing. The pH adjustment is carried out by using an organic or inorganic acid, an alkali solution, urea, calcium carbonate, ammonia, etc.

If necessary, antibiotics such as ampicillin and tetracycline may be added to the medium during the culturing.

When a microorganism transformed with an expression vector comprising an inducible promoter is cultured, an inducer may be added to the medium, if necessary. For example, in the case of a microorganism transformed with an expression vector comprising lac promoter, isopropyl-β-D-thiogalactopyranoside or the like may be added to the medium; and in the case of a microorganism transformed with an expression vector comprising trp promoter, indoleacrylic acid or the like may be added.

For the culturing of the transformant obtained by using an animal cell as the host cell, generally employed media such as RPMI1640 medium [J. Am. Med. Assoc., 199, 519 (1967)], Eagle's MEM [Science, 122, 501 (1952)], DMEM [Virology, 8, 396 (1959)] and 199 medium [Proc. Soc. Biol. Med., 73, 1 (1950)], media prepared by adding fetal calf serum or the like to these media, etc. can be used as the medium.

Culturing is usually carried out at pH 6 to 8 at 25 to 40° C. for 1 to 7 days in the presence of 5% CO₂.

If necessary, antibiotics such as kanamycin, penicillin and streptomycin may be added to the medium during the culturing.

For the culturing of the transformant obtained by using an insect cell as the host cell, generally employed media such as TNM-FH medium (PharMingen, Inc.), Sf-900 II SFM medium (Life Technologies, Inc.), ExCell 400 and ExCell 405 (JRH Biosciences, Inc.) and Grace's Insect Medium [Nature, 195, 788 (1962)] can be used as the medium.

Culturing is usually carried out at pH 6 to 7 at 25 to 30° C. for 1 to 5 days.

If necessary, antibiotics such as gentamicin may be added to the medium during the culturing.

The transformant obtained by using a plant cell as the host cell may be cultured in the form of cells as such or after differentiation into plant cells or plant organs. For the culturing of such transformant, generally employed media such as Murashige-Skoog (MS) medium and White medium, media prepared by adding phytohormones such as auxin and cytokinin to these media, etc. can be used as the medium.

Culturing is usually carried out at pH 5 to 9 at 20 to 40° C. for 3 to 60 days.

If necessary, antibiotics such as kanamycin and hygromycin may be added to the medium during the culturing.

As described above, the protein used in the present invention can be produced by culturing the transformant derived from microorganisms, insect cells, animal cells or plant cells, and carrying the recombinant DNA wherein the DNA used in the present invention is ligated to an expression vector, allowing the protein used in the present invention to form and accumulate in the culture, and recovering the protein from the culture.

The protein used in the present invention may be produced by intracellular production by host cells, extracellular secretion by host cells or production on outer membranes by host cells. A desirable production method can be adopted by changing the kind of the host cells used or the structure of the protein to be produced.

When the protein used in the present invention is produced in host cells or on outer membranes of host cells, it is possible to force the protein to be secreted outside the host cells by applying the method of Paulson, et al. [J. Biol. Chem., 264, 17619 (1989)], the method of Lowe, et al. [Proc. Natl. Acad. Sci. USA, 86, 8227 (1989); Genes Develop., 4, 1288 (1990)], or the methods described in Japanese Published Unexamined Patent Application No. 336963/93, WO94/23021, etc.

That is, extracellular secretion of the protein of the present invention by host cells can be caused by expressing it in the form of a protein in which a signal peptide is added upstream of a protein containing the active site of the protein of the present invention by the use of recombinant DNA techniques.

It is also possible to increase the protein production by utilizing a gene amplification system using a dihydrofolate reductase gene or the like according to the method described in Japanese Published Unexamined Patent Application No. 227075/90.

Further, the protein used in the present invention can be produced using an animal having an introduced gene (non-human transgenic animal) or a plant having an introduced gene (transgenic plant) constructed by redifferentiation of animal or plant cells carrying the introduced gene.

When the transformant producing the protein used in the present invention is an animal or plant, the protein can be produced by raising or culturing the animal or plant in a usual manner, allowing the protein to form and accumulate therein, and recovering the protein from the animal or plant.

Production of the protein used in the present invention using an animal can be carried out, for example, by producing the protein in an animal constructed by introducing the gene according to known methods [Am. J. Clin. Nutr., 63, 639S (1996); Am. J. Clin. Nutr., 63, 627S (1996); Bio/Technology, 9, 830 (1991)].

In the case of an animal, the protein used in the present invention can be produced, for example, by raising a non-human transgenic animal carrying the introduced DNA used in the present invention, allowing the protein to form and accumulate in the animal, and recovering the protein from the animal. The places where the protein is formed and accumulated include milk (Japanese Published Unexamined Patent Application No. 309192/88), egg, etc. of the animal. As the promoter in this process, any promoters capable of functioning in an animal can be used. Preferred promoters include mammary gland cell-specific promoters such as α casein promoter, βcasein promoter, β lactoglobulin promoter and whey acidic protein promoter.

Production of the protein used in the present invention using a plant can be carried out, for example, by culturing a transgenic plant carrying the introduced DNA encoding the protein used in the present invention according to known methods [Soshiki Baiyo (Tissue Culture), 20, (1994); Soshiki Baiyo, 21, (1995); Trends Biotechnol., 15, 45 (1997)], allowing the protein to form and accumulate in the plant, and recovering the protein from the plant.

The protein-used in the present invention produced by using the transformant producing the protein can be isolated and purified by conventional methods for isolating and purifying enzymes.

For example, when the protein used in the present invention is produced in a soluble form in cells, the cells are recovered by centrifugation after the completion of culturing and suspended in an aqueous buffer, followed by disruption using a sonicator, French press, Manton Gaulin homogenizer, Dynomill or the like to obtain a cell-free extract.

A purified protein preparation can be obtained by centrifuging the cell-free extract to obtain the supernatant and then subjecting the supernatant to ordinary means for isolating and purifying enzymes, e.g., extraction with a solvent, salting-out with ammonium sulfate, etc., desalting, precipitation with an organic solvent, anion exchange chromatography using resins such as diethylaminoethyl (DEAE)-Sepharose and DIAION HPA-75 (Mitsubishi Chemical Corporation), cation exchange chromatography using resins such as S-Sepharose FF (Pharmacia), hydrophobic chromatography using resins such as butyl Sepharose and phenyl Sepharose, gel filtration using a molecular sieve, affinity chromatography, chromatofocusing, and electrophoresis such as isoelectric focusing, alone or in combination.

When the protein is produced as an inclusion body in cells, the cells are similarly recovered and disrupted, followed by centrifugation to obtain a precipitate fraction. After the protein is recovered from the precipitate fraction by an ordinary method, the inclusion body of the protein is solubilized with a protein-denaturing agent.

The solubilized protein solution is diluted with or dialyzed against a solution containing no protein-denaturing agent or a solution containing the protein-denaturing agent at such a low concentration that denaturation of protein is not caused, whereby the protein is renatured to have normal higher-order structure. Then, a purified protein preparation can be obtained by the same isolation and purification steps as described above.

When the protein used in the present invention or its derivative such as a glycosylated form is extracellularly secreted, the protein or its derivative such as a glycosylated form can be recovered in the culture supernatant.

That is, the culture is treated by the same means as above, e.g., centrifugation, to obtain a soluble fraction. A purified protein preparation can be obtained from the soluble fraction by using the same isolation and purification methods as described above.

Examples of the proteins obtained in the above manner are proteins respectively consisting of the amino acid sequences shown in SEQ ID NOS: 1 to 8, 37 and 38.

It is also possible to produce the protein used in the present invention as a fusion protein with another protein and to purify it by affinity chromatography using a substance having affinity for the fused protein.

Examples of the proteins to be fused include β-galactosidase, protein A, immunoglobulin G-binding region of protein A, chloramphenicol acetyltransferase, poly(Arg), poly(Glu), protein G, maltose-binding protein, glutathione S-transferase, polyhistidine chain (His-tag), S peptide, DNA-binding protein domain, Tac antigen, thioredoxin, green fluorescent protein, FLAG peptide and arbitrary antibody epitopes [Akio Yamakawa, Experimental Medicine, 13, 469-474 (1995)].

Examples of the substances having affinity for the above proteins to be fused include antibodies recognizing β-galactosidase, protein A, immunoglobulin G-binding region of protein A, chloramphenicol acetyltransferase, poly(Arg), poly(Glu), protein G, maltose-binding protein, glutathione S-transferase, polyhistidine chain (His-tag), S peptide, DNA-binding protein domain, Tac antigen, thioredoxin, green fluorescent protein, FLAG peptide and arbitrary antibody epitopes, such as immunoglobulin G.

Specifically, when the protein used in the present invention is produced as a fusion protein with protein A, the fusion protein can be purified according to the method of Lowe, et al. [Proc. Natl. Acad. Sci. USA, 86, 8227 (1989); Genes Develop., 4, 1288 (1990)] and the methods described in Japanese Published Unexamined Patent Application No. 336963/93 and WO94/23021. When the protein used in the present invention is produced as a fusion protein with a Flag peptide, the fusion protein can be purified according to the methods described in Proc. Natl. Acad. Sci. USA, 86, 8227 (1989); Genes Develop., 4, 1288 (1990), etc. The protein can also be purified by affinity chromatography using an antibody against said protein.

The protein used in the present invention can also be produced by chemical synthetic methods such as the Fmoc method (the fluorenylmethyloxycarbonyl method) and the tBoc method (the t-butyloxycarbonyl method) based on the amino acid information on the protein obtained above. Further, the protein can be chemically synthesized by using peptide synthesizers from Advanced ChemTech, Perkin-Elmer, Pharmacia, Protein Technology Instrument, Synthecell-Vega, PerSeptive, Shimadzu Corporation, etc.

### 6. Process for Producing Dipeptide Derivatives of the Present Invention

### (1) The Production Process by the Enzymatic Method

The production processes of dipeptide derivatives by the enzymatic method include:

i) a process for producing dipeptide or dipeptide derivative PI, which comprises:
allowing the protein above 1, one or more kinds of amino acids or amino acid derivatives and ATP to be present in an aqueous medium;
allowing dipeptide or dipeptide derivative PI to form and accumulate in the aqueous medium; and
recovering dipeptide or dipeptide derivative PI from the medium; and
ii) a process for producing dipeptide or dipeptide derivative PII, which comprises:
allowing the protein above 1, one or more kinds of amino acids or amino acid derivatives and ATP to be present in an aqueous medium;
allowing dipeptide or dipeptide derivative PI to form and accumulate in the aqueous medium;
subjecting the dipeptide or dipeptide derivative PI, as such or after recovery, to modification to form the dipeptide or dipeptide derivative PII; and
recovering the dipeptide or dipeptide derivative PII.

Modification of dipeptide or dipeptide derivative PI to form dipeptide or dipeptide derivative PII can be carried out by known organic synthesis techniques.

In the process above, as one or more kinds of amino acids or amino acid derivatives, any amino acid and amino acid derivative that can be used as the substrate of the protein of above 1 can be used as a substrate.

The above amino acids or amino acid derivatives include amino acids or amino acid derivatives represented by formula (I):

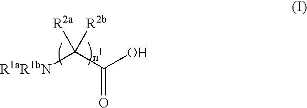

(wherein n¹, R^{1a}, R^{1b}, R^{2a} and R^{2b} have the same significances as defined above) or formula (II):

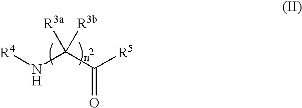

(wherein n², R^{3a}, R^{3b}, R⁴ and R⁵ have the same significances as defined above), provided that when all the amino acids or amino acid derivatives used are amino acids or amino acid derivatives represented by formula (I), at least one of R^{1a} and R^{1b} is a hydrogen atom, and when all the amino acids or amino acid derivatives used are amino acids or amino acid derivatives represented by formula (II), R⁵ is hydroxy. Preferred amino acids or amino acid derivatives are those represented by formula (III):

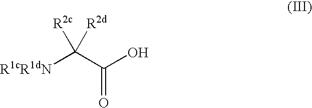

(wherein R^{1c}, R^{1d}, R^{2c} and R^{2d} have the same significances as defined above) or formula (IV):

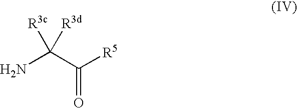

(wherein R^{3c}, R^{3d} and R⁵ have the same significances as defined above), provided that when all the amino acids or amino acid derivatives used are amino acids or amino acid derivatives represented by formula (III), at least one of R^{1c} and R^{1d} is a hydrogen atom, and when all the amino acids or amino acid derivatives used are amino acids or amino acid derivatives represented by formula (IV), R⁵ is hydroxy. More preferred amino acids or amino acid derivatives are those represented by formula (V):

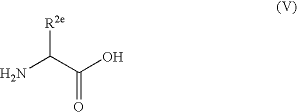

(wherein R^{2e} has the same significance as defined above) or formula (VI):

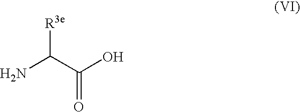

(wherein R^{3e} has the same significance as defined above).

Examples of dipeptide or dipeptide derivative PI produced by the above process include dipeptides or dipeptide derivatives represented by formula (VIIa):

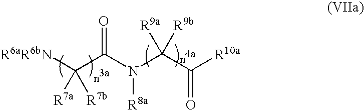

(wherein n^{3a}, n^{4a}, R^{6a}, R^{6b}, R^{7a}, R^{7b}, R^{8a}, R^{9a}, R^{9b} and R^{10a} have the same significances as defined above), preferably, dipeptides or dipeptide derivatives represented by formula (VIIIa):

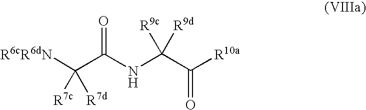

(wherein R^{6c}, R^{6d}, R^{7c}, R^{7d}, R^{9c}, R^{9d} and R^{10a} have the same significances as defined above), more preferably, dipeptides or dipeptide derivatives represented by formula (IXa):

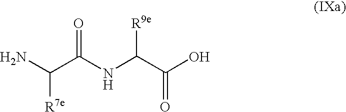

(wherein R^{7e} and R^{9e} have the same significances as defined above).

Examples of dipeptide or dipeptide derivative PII produced by the above process include dipeptides or dipeptide derivatives represented by formula (VIIb):

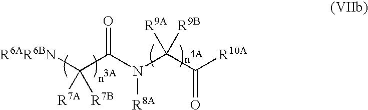

(wherein n^{3A}, n^{4A}, R^{6A}, R^{6B}, R^{7A}, R^{7B}, R^{8A}, R^{9A}, R^{9B} and R^{10A} have the same significances as defined above), preferably, dipeptides or dipeptide derivatives represented by formula (VIIIb):

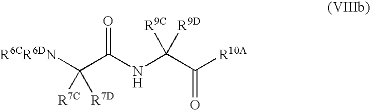

(wherein R^{6C}, R^{6D}, R^{7C}, R^{7D}, R^{9C}, R^{9D} and R^{10A} have the same significances as defined above), more preferably, dipeptides or dipeptide derivatives represented by formula (IXb):

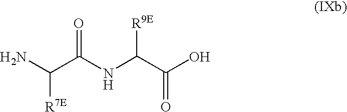

(wherein R^{7E} and R^{9E} have the same significances as defined above). The above dipeptides or dipeptide derivatives do not include compounds in which the same or different amino acids selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine, L-citrulline, L-6-diazo-5-oxo-norleucine, glycine and β-alanine are bound by peptide bond.

In the definitions of the groups in formulae (I) to (VI), (VIIa), (VIIb), (VIIIa), (VIIIb), (IXa) and (IXb), the lower alkyl moiety of the lower alkyl, the lower alkoxy, the lower alkanoyl, the lower alkoxycarbonyl, the mono(lower alkyl)amino and the di(lower alkyl)amino includes alkyl groups with 1 to 10 carbon atoms having a linear-structure, a branched-chain structure, a cyclic structure or a combination thereof. Examples of the linear or branched-chain alkyl groups are methyl, ethyl, n-propyl, isopropyl, n-butyl, isobutyl, sec-butyl, tert-butyl, n-pentyl, neopentyl, n-hexyl, n-heptyl, n-octyl, n-nonyl and n-decyl. Examples of the cyclic alkyl groups are cyclopropyl, cyclobutyl, cyclopentyl, cyclohexyl, cycloheptyl, cyclooctyl, cyclodecyl, noradamantyl, adamantyl, bicyclo[2.2.1]heptyl, bicyclo[2.2.2]octyl, bicyclo[3.3.0]octyl and bicyclo[3.3.1]nonyl. Examples of the alkyl groups having a combination of a linear or branched-chain structure and a cyclic structure are cyclopropylmethyl, cyclopentylmethyl and cyclooctylethyl. Two lower alkyl moieties of the di(lower alkyl)amino may be the same or different.

The lower alkenyl includes linear or branched-chain alkenyl groups having 2 to 10 carbon atoms, such as vinyl, allyl, 1-propenyl, 1-butenyl, 3-butenyl, 2-pentenyl, 4-pentenyl, 2-hexenyl, 5-hexenyl, 2-decenyl and 9-decenyl.

The lower alkynyl includes linear or branched-chain alkynyl groups having 2 to 10 carbon atoms, such as ethynyl, 2-propynyl, 3-butynyl, 4-pentynyl, 5-hexynyl and 9-decynyl.

The aryl moiety of the aryl, the aralkyl and the aroyl includes monocyclic aryl groups and condensed-ring aryl groups in which two or more rings are condensed, specifically, aryl groups having 6 to 14 ring-constituting carbon atoms, such as phenyl, naphthyl, indenyl and anthranyl.

The alicyclic heterocyclic group includes monocyclic ones and condensed-ring ones in which two or more rings are condensed. Though the kind and number of heteroatoms contained in the alicyclic heterocyclic group are not specifically limited, the alicyclic heterocyclic group may contain, for example, one or more heteroatoms selected from the group consisting of nitrogen atom, sulfur atom and oxygen atom. Specific examples of the alicyclic heterocyclic groups are pyrrolidinyl, 2,5-dioxopyrrolidinyl, thiazolidinyl, oxazolidinyl, piperidyl, 1,2-dihydropyridyl, piperazinyl, homopiperazinyl, morpholinyl, thiomorpholinyl, pyrazolinyl, oxazolinyl, dioxolanyl, tetrahydropyranyl, tetrahydrothiopyranyl, tetrahydrofuryl, tetrahydroquinolyl, tetrahydroisoquinolyl, tetrahydroquinoxalinyl, octahydroquinolyl, dihydroindolyl and 1,3-dioxoisoindolinyl.

The heterocyclic group moiety of the heterocyclic alkyl includes, for example, aromatic heterocyclic groups and alicyclic heterocyclic groups. The aromatic heterocyclic groups include monocyclic ones and condensed-ring ones in which two or more rings are condensed. Though the kind and number of heteroatoms contained in the aromatic heterocyclic group are not specifically limited, the aromatic heterocyclic group may contain, for example, one or more heteroatoms selected from the group consisting of nitrogen atom, sulfur atom and oxygen atom. Specific examples of the aromatic heterocyclic groups are those having 5 to 14 ring-constituting atoms, such as furyl, thienyl, pyrrolyl, imidazolyl, pyrazolyl, triazolyl, tetrazolyl, oxazolyl, oxadiazolyl, thiazolyl, pyridyl, pyrazinyl, pyrimidinyl, pyridazinyl, triazinyl, indolyl, indazolyl, benzimidazolyl, benzoxazolyl, benzothiazolyl, quinolyl, isoquinolyl, phthalazinyl, naphthyridinyl, quinoxalinyl, quinazolinyl, cinnolinyl, purinyl and coumarinyl. The alicyclic heterocyclic group has the same significance as defined above.

The alkylene moiety of the aralkyl and the heterocyclic alkyl has the same significance as the linear or branched-chain alkyl among the above-described lower alkyl groups except one hydrogen atom is removed therefrom.

The heterocyclic group formed together with the adjacent nitrogen atom and the carbon atom adjacent to the nitrogen atom and the heterocyclic group formed together with the adjacent carbon atom and the nitrogen atom adjacent to the carbon atom include 5- or 6-membered monocyclic alicyclic heterocyclic groups containing at least one nitrogen atom (the monocyclic alicyclic heterocyclic groups may also contain another nitrogen atom, oxygen atom or sulfur atom), and bicyclic or tricyclic condensed-ring heterocyclic groups containing at least one nitrogen atom in which 3- to 8-membered rings are condensed (the condensed-ring heterocyclic groups may also contain another nitrogen atom, oxygen atom or sulfur atom), specifically, pyrrolidinyl, piperidyl, piperazinyl, morpholinyl, thiomorpholinyl, homopiperidinyl, homopiperazinyl, tetrahydropyridyl, tetrahydroquinolyl and tetrahydroisoquinolyl.

The substituted lower alkyl, the substituted lower alkenyl, the substituted lower alkynyl, the substituted lower alkoxy, the substituted lower alkanoyl, the substituted lower alkoxycarbonyl, the substituted aralkyl, the substituted aryl, the substituted aroyl, the substituted heterocyclic alkyl, the mono(substituted lower alkyl)amino, the lower alkyl(substituted lower alkyl)amino, the di(substituted lower alkyl)amino, the substituted heterocyclic group formed together with the adjacent nitrogen atom and the carbon atom adjacent to the nitrogen atom, and the substituted heterocyclic group formed together with the adjacent carbon atom and the nitrogen atom adjacent to the carbon atom each have one to a substitutable number, preferably 1 to 3 substituents which are the same or different. Examples of the substituents include halogen, amino, nitro, hydroxy, mercapto, guanidino, ureido, cyano, formyl, carboxyl, aminocarbonyl, diazoacetyl, lower alkyl, lower alkoxy, lower alkanoyl, lower alkoxycarbonyl, mono or di(lower alkyl)aminocarbonyl, lower alkylthio, aryl, aralkyl, aroyl and heterocyclic carbonyl. The lower alkyl moiety of the lower alkyl, the lower alkoxy, the lower alkanoyl and the lower alkoxycarbonyl, the aryl moiety of the aryl, the aralkyl and the aroyl, and the alkylene moiety of the aralkyl have the same significances as defined above, respectively. The lower alkyl moiety of the mono or di(lower alkyl)aminocarbonyl and the lower alkylthio has the same significance as the above lower alkyl, and the heterocyclic group moiety of the heterocyclic carbonyl has the same significance as the heterocyclic group moiety of the above heterocyclic alkyl. The halogen includes fluorine, chlorine, bromine and iodine atoms. Two lower alkyl moieties of the di(lower alkyl)aminocarbonyl may be the same or different.

Preferred amino acids or amid acid derivatives above include those selected from the group consisting of L-amino acids, glycine (Gly), β-alanine (βAla) and their derivatives. Examples of L-amino acids are L-alanine (L-Ala), L-glutamine (L-Gln), L-glutamic acid (L-Glu), L-valine (L-Val), L-leucine (L-Leu), L-isoleucine (L-Ile), L-proline (L-Pro), L-phenylalanine (L-Phe), L-tryptophan (L-Trp), L-methionine (L-Met), L-serine (L-Ser), L-threonine (L-Thr), L-cysteine (L-Cys), L-asparagine (L-Asn), L-tyrosine (L-Tyr), L-lysine (L-Lys), L-arginine (L-Arg), L-histidine (L-His), L-aspartic acid (L-Asp), L-α-aminobutyric acid (L-α-AB), L-azaserine, L-theanine, L-4-hydroxyproline (L-4-HYP), L-3-hydroxyproline (L-3-HYP), L-ornithine (L-Orn), L-citrulline (L-Cit) and L-6-diazo-5-oxo-norleucine.

Combinations of amino acids or amino acid derivatives used in the above production processes include the following: a combination of one kind of amino acid selected from the group consisting of L-Ala, Gly, L-Met, L-Ser, L-Thr and β-Ala, or its derivative, and one kind of amino acid selected from the group consisting of L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-azaserine, L-theanine, L-4-HYP, L-3-HYP, L-Orn, L-Cit and L-6-diazo-5-oxo-norleucine, or its derivative; a combination of L-Gln or its derivative and L-Phe or its derivative; and a combination of L-α-AB or its derivative, and one kind of amino acid selected from the group consisting of L-Gln, L-Arg and L-α-AB, or its derivative. Further preferred combinations are: a combination of L-Ala or its derivative, and one kind of amino acid selected from the group consisting of L-Gln, Gly, L-Val, L-Leu, L-Ile, L-Phe, L-Trp, L-Met, L-Ser, L-Thr, L-Cys, L-Asn, L-Tyr, L-Lys, L-Arg, L-His, L-α-AB, L-azaserine, L-Cit and L-theanine, or its derivative; a combination of Gly or its derivative, and one kind of amino acid selected from the group consisting of L-Gln, Gly, L-Phe, L-Trp, L-Met, L-Ser, L-Thr, L-Cys, L-Tyr, L-Lys, L-Arg, L-α-AB and L-Cit, or its derivative; a combination of L-Met or its derivative, and one kind of amino acid selected from the group consisting of L-Phe, L-Met, L-Ser, L-Thr, L-Cys, L-Tyr, L-Lys and L-His, or its derivative; a combination of L-Ser or its derivative, and one kind of amino acid selected from the group consisting of L-Gln, L-Phe, L-Ser, L-Thr, L-Tyr, L-His and L-α-AB, or its derivative; a combination of L-Thr or its derivative, and one kind of amino acid selected from the group consisting of L-Gln, L-Phe, L-Leu, L-Thr and L-α-AB, or its derivative; a combination of L-Gln or its derivative and L-Phe orbits derivative; a combination of β-Ala or its derivative, and one kind of amino acid selected from the group consisting of L-Phe, L-Met, L-His and L-Cit, or its derivative; and a combination of L-α-AB or its derivative, and one kind of amino acid selected from the group consisting of L-Gln, L-Arg and L-α-AB, or its derivative.

The amount of the protein used in the present invention in the aqueous medium is 0.01 to 100 mg, preferably 0.1 to 10 mg per mg of amino acid or amino acid derivative used as a substrate.

In the above production processes, the amino acids or amino acid derivatives used as substrates are added to the aqueous medium at the start or in the course of reaction to give a concentration of 0.1 to 500 g/l, preferably 0.2 to 200 g/l.

In the above production processes, ATP used as energy source is added to the aqueous medium at the concentration of 0.5 mmol/l to 10 mol/l.

The aqueous medium used in the above production processes may comprise any components and may have any composition so far as the dipeptide- or dipeptide derivative-forming reaction is not inhibited. Suitable aqueous media include water and buffers such as phosphate buffer, carbonate buffer, acetate buffer, borate buffer, citrate buffer and Tris buffer. The aqueous medium may comprise alcohols such as methanol and ethanol, esters such as ethyl acetate, ketones such as acetone, and amides such as acetamide.

The dipeptide-forming reaction is carried out in the aqueous medium at pH 5 to 11, preferably pH 6 to 10, at 20 to 50° C., preferably 25 to 45° C., for 2 to 150 hours, preferably 6 to 120 hours.

### (2) The Production Process Using the Culture of the Cells or the Treated Matters of the Culture as an Enzyme Source

The production processes of dipeptides or dipeptide derivatives using the culture of the cells or the treated matters of the culture as an enzyme source include:

i) the process for the production of a dipeptide or a dipeptide derivative PI which comprise:
using the culture of the cells or the treated matters of the culture obtained by the method of above 4 as an enzyme source;
allowing the enzyme source and one or more kinds of amino acids of amino acid derivatives to be present in an aqueous medium;
allowing the dipeptide or dipeptide derivative PI to form and accumulate in the aqueous medium; and
recovering the dipeptide or dipeptide derivative PI from the medium; and
ii) the process for the production of dipeptides or dipeptide derivatives which comprises:
using the culture of cells or the treated matters of the culture as an enzyme source;
allowing the enzyme source and one or more kinds of amino acids or amino acid derivatives to be present in an aqueous medium;
allowing the dipeptide or dipeptide derivative PI to form and accumulate in the medium;
subjecting the dipeptide or dipeptide derivative PI, as such or after recovery, to modification to form dipeptide or dipeptide derivative PII; and
recovering the dipeptide or dipeptide derivative PII.

Modification of dipeptide or dipeptide derivative PI to form dipeptide or dipeptide derivative PII can be carried out by known organic synthesis techniques.

In the production process above, species of amino acids and amino acid derivatives used as substrates, the concentration and the time of addition of the substrate and dipeptide which can produced are the same as the enzymatic method above 6(1).

The treated matters of the culture include treated matters which are selected from the group consisting of concentrated culture, dried culture, cells obtained by centrifuging the culture, dried cells, freeze-dried cells, surfactant-treated cells, ultrasonicated cells, mechanically-disrupted cells, solvent-treated cells, enzymatic-treated cells, protein fractionation of cells, immobilized cells, and an enzyme preparation obtained by extracting the cells, and which have the activity to form dipeptides or dipeptide derivatives from one or more kinds of amino acids or amino acid derivatives.

The culture of cells used as an enzyme source also used as an aqueous medium in addition to the aqueous medium used in the enzymatic method above 6(1) in the production process using the culture of the cells or the treated matters of the culture as an enzyme source.

In the above production processes, as sources of ATP, ATP or substances from which cells can produce ATP by the metabolism, for example, sugars such as glucose, alcohols such as ethanol, and organic acids such as acetic acid, can be used instead of ATP according to need.

Further, a surfactant or an organic solvent may be added to the aqueous medium according to need. Suitable surfactants include nonionic surfactants such as polyoxyethylene octadecylamine (e.g., Nymeen S-215, NOF Corporation), cationic surfactants such as cetyltrimethylammonium bromide and alkyldimethylbenzylammonium chloride (e.g., Cation F2-40E, NOF Corporation), anionic surfactants such as lauroyl sarcosinate, and tertiary amines such as alkyldimethylamine (e.g., Tertiary Amine FB, NOF Corporation). Suitable organic solvents include xylene, toluene, aliphatic alcohols, acetone and ethyl acetate. Any surfactants and organic solvents may be used alone or in combination provided that the cells used in the production processes of the present invention have the activity to form dipeptide or dipeptide derivative PI from amino acids or amino acid derivatives. The kind and concentration of the surfactant and organic solvent can be arbitrarily selected so long as the cells used in the present invention have the above activity. For example, the surfactant is usually used at a concentration of 0.1 to 50 g/l, and the organic solvent is usually used at a concentration of 0.1 to 50 ml/l.

When the culture of cells or a treated matter of the culture is used as the enzyme source, the amount of the enzyme source to be added varies according to its specific activity, etc., but is, for example, 5 to 1000 mg (wet cell weight), preferably 10 to 400 mg per mg of amino acid or amino acid derivative used as a substrate.

The dipeptide- or dipeptide derivative-forming reaction is carried out in the aqueous medium at pH 5 to 11, preferably pH 6 to 10, at 20 to 65° C., preferably 25 to 55° C., more preferably 30 to 45° C., usually for 1 minute to 150 hours, preferably 3 minutes to 120 hours, more preferably 30 minutes to 100 hours.

In the above-production processes of 6(1) or (2), recovery of the dipeptide or dipeptide derivative formed and accumulated in the aqueous medium can be carried out by ordinary methods using active carbon, ion-exchange resins, etc. or by means such as extraction with an organic solvent, crystallization, thin layer chromatography and high performance liquid chromatography.

### (3) The Production Process for Dipeptide Derivative by Modifying Dipeptide or Dipeptide Derivative

Various dipeptide derivatives can be obtained by subjecting the dipeptide or dipeptide derivative obtained in the above 6(1) or (2) to known organic synthesis method [see Comprehensive Organic Transformations, R. C. Larock, 1989, for example] and the like.

The product of the production process above can be isolated or purified by the method of the usual organic synthesis like filtration, extraction, washing, drying, condensation, crystallization, various chromatography or the appropriate combination thereof.

The methods for preparing the DNA, protein and cells used in the present invention are illustrated in the following experimental examples, but the preparation methods are not limited to the following experimental examples.

### EXPERIMENTAL EXAMPLE 1

### Search for a Protein Having the Dipeptide-Synthesizing Activity Utilizing a Database

By using, as a query, the amino acid sequence of D-Ala-D-Ala ligase gene derived from Bacillus subtilis 168 [Nature, 390, 249-256 (1997)], a search for a gene encoding a protein having homology which is present in the genomic DNA sequences of Bacillus subtilis 168 was carried out using the homology search function of Subtilist (http://genolist.pasteur.fr/SubtiList/) which is a database of the genomic DNA of Bacillus subtilis 168.

From the sequences obtained as a result of the search, genes encoding the amino acid sequences shown in SEQ ID NOS: 33, 34 and 35 which are D-Ala-D-Ala ligase motifs [Biochemistry, 30, 1673 (1991)] and encoding proteins whose function had already been clarified were excluded. Of the remaining sequences, the sequence showing the highest homology (29.1%) to the D-Ala-D-Ala ligase motif was selected as a gene of unknown function ywfE.

The nucleotide sequence of ywfE is shown in SEQ ID NO: 9, and the amino acid sequence of the protein encoded by the nucleotide sequence is shown in SEQ ID NO: 1.

### EXPERIMENTAL EXAMPLE 2

### Construction of a Strain Expressing ywfE Gene

On the basis of the information on the nucleotide sequence obtained in Experimental Example 1, a ywfE gene fragment of Bacillus subtilis was obtained in the following manner.

That is, Bacillus subtilis 168 (ATCC 23857) was inoculated into LB medium [10 g/l Bacto-tryptone (Difco), 5 g/l yeast extract (Difco) and 5 g/l sodium chloride] and subjected to static culture overnight at 30° C. After the culturing, the chromosomal DNA of the microorganism was isolated and purified according to the method using saturated phenol described in Current Protocols in Molecular Biology.

By using a DNA synthesizer (Model 8905, PerSeptive Biosystems, Inc.), DNAs having the nucleotide sequences shown in SEQ ID NOS: 19 to 22 (hereinafter referred to as primer A, primer B, primer C and primer D, respectively) were synthesized. Primer A has a sequence wherein a nucleotide sequence containing the XhoI recognition sequence is added to the 5′ end of a region of the Bacillus subtilis chromosomal DNA containing the initiation codon of ywfE. Primer B has a sequence wherein a nucleotide sequence containing the BamHI recognition sequence is added to the 5′ end of a nucleotide sequence complementary to a sequence containing the termination codon of ywfE. Primer C has a sequence wherein a nucleotide sequence containing the EcoRI recognition sequence is added to the 5′ end of the nucleotide sequence of trp promoter region of expression vector pTrS30 containing trp promoter [prepared from Escherichia coli JM109/pTrS30 (FERM BP-5407)]. Primer D has a sequence wherein a nucleotide sequence containing the XhoI recognition sequence is added to the 5′ end of a sequence complementary to the sequence of trp promoter region of expression vector pTrS30 containing trp promoter.

A ywfE gene fragment was amplified by PCR using the above primer A and primer B, and as a template, the chromosomal DNA of Bacillus subtilis. A trp promoter region fragment was amplified by PCR using primer C and primer D, and as a template, pTrS30. PCR was carried out by 30 cycles, one cycle consisting of reaction at 94° C. for one minute, reaction at 55° C. for 2, minutes and reaction at 72° C. for 3 minutes, using 40 μl of a reaction mixture comprising 0.1 μg of the chromosomal DNA or 10 ng of pTrS30 as a template, 0.5 μmol/l each of the primers, 2.5 units of Pfu DNA polymerase (Stratagene), 4 μl of buffer for Pfu DNA polymerase (10×) (Stratagene) and 200 μmol/l each of dNTPs (DATP, dGTP, dCTP and dTTP).

One-tenth of each of the resulting reaction mixtures was subjected to agarose gel electrophoresis to confirm that a ca. 1.4 kb DNA fragment corresponding to the ywfE gene fragment and a ca. 0.3 kb DNA fragment corresponding to the trp promoter region fragment were respectively amplified in the PCR using primer A and primer B and the PCR using primer C and primer D. Then, the remaining reaction mixture was mixed with an equal amount of phenol/chloroform (1 vol/1 vol) saturated with TE [10 mmol/l Tris-HCl (pH 8.0), 1 mmol/l EDTA]. The resulting solution was centrifuged, and the obtained upper layer was mixed with a two-fold volume of cold ethanol and allowed to stand at −80° C. for 30 minutes. The resulting solution was centrifuged to precipitate DNA, and the obtained DNA was dissolved in 20 μl of TE.

The thus obtained solutions (5 μl each) were respectively subjected to reaction to cleave the DNA amplified using primer A and primer B with restriction enzymes XhoI and BamHI and to reaction to cleave the DNA amplified using primer C and primer D with restriction enzymes EcoRI and XhoI. DNA fragments were separated by agarose gel electrophoresis, and a 1.4 kb fragment containing ywfE and a 0.3 kb fragment containing trp promoter region were respectively recovered using GENECLEAN II Kit (BIO 101).

pTrS30 [a trp promoter-containing expression vector prepared from Escherichia coli JM109/pTrS30 (FERM BP-5407), 0.2 μg] was cleaved with restriction enzymes EcoRI and BamHI. DNA fragments were separated by agarose gel electrophoresis and a 4.5 kb DNA fragment was recovered in the same manner as above.

The 1.4 kb fragment containing ywfE, the 0.3 kb fragment containing trp promoter region and the 4.5 kb DNA fragment obtained above were subjected to ligation reaction using a ligation kit (Takara Bio Inc.) at 16° C. for 16 hours.

Escherichia coli NM522 (Stratagene) was transformed using the reaction mixture according to the method using calcium ion [Proc. Natl. Acad. Sci. USA, 69, 2110 (1972)], spread on LB agar medium containing 50 μg/ml ampicillin, and cultured overnight at 30° C.

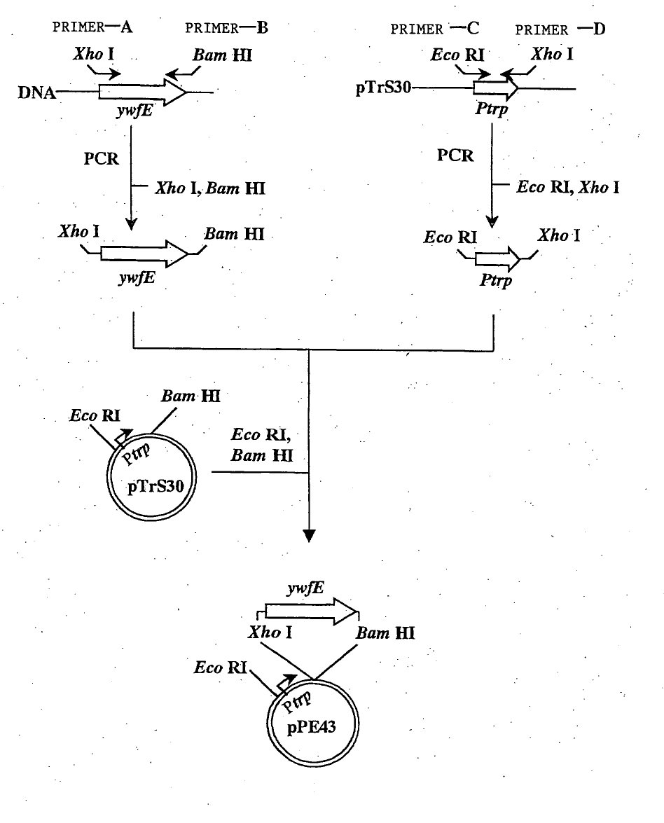

A plasmid was extracted from a colony of the transformant that grew on the medium according to a known method and the structure of the plasmid was analyzed using restriction enzymes, whereby it was confirmed that expression vector pPE43 containing ywfE ligated downstream of the trp promoter was obtained (FIG. 1).

### EXPERIMENTAL EXAMPLE 3

### Production of a Dipeptide

Escherichia coli NM522 carrying pPE43 (Escherichia coli NM522/pPE43) obtained in Experimental Example 2 was inoculated into 8 ml of LB medium containing 50 μg/ml ampicillin in a test tube, and cultured at 28° C. for 17 hours. The resulting culture was centrifuged to obtain wet cells.

A reaction mixture (0.1 ml) comprising 60 mg/ml (final concentration) wet cells, 120 mmol/l potassium phosphate buffer (pH 7.4), 60 mmol/1 magnesium chloride, 60 mmol/l ATP, 30 mmol/l L-Ala, 30 mmol/l L-Gln and 0.4% Nymeen S-215 was prepared, and reaction was carried out at 37° C. for 3 minutes.

After the completion of reaction, the reaction product was derivatized by the dinitrophenol method and then analyzed by HPLC. The HPLC analysis was carried out using, as a separation column, Lichrosorb-RP-18 column (Kanto Kagaku) and, as an eluent, 1% (v/v) phosphoric acid and 25% (v/v) acetonitrile at a flow rate of 0.7 ml/min. As a result, it was confirmed that 120 mg/l L-alanyl-L-glutamine (L-Ala-L-Gln) was formed and accumulated in the reaction mixture.

Formation of L-Ala-L-Gln was not observed when the reaction was carried out using cells of Escherichia coli NM522/pTrS31, which is a control strain carrying only a vector.

### EXPERIMENTAL EXAMPLE 4

### Purification of C-Terminal His-Tagged Recombinant Dipeptide Synthetase

By using the above DNA synthesizer, DNAs having the nucleotide sequences shown in SEQ ID NOS: 23 and 24 (hereinafter referred to as primer E and primer F, respectively) were synthesized. Primer E has a nucleotide sequence containing a region wherein the initiation codon of ywfE (atg) is substituted by the NcoI recognition sequence (ccatgg). Primer F has a nucleotide sequence containing a region wherein the termination codon of ywfE is substituted by the BamHI recognition sequence (ggatcc).

PCR was carried out using the chromosomal DNA of Bacillus subtilis 168 (ATCC 23857) as a template and the above primer E and primer F as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

One-tenth of the resulting reaction mixture was subjected to agarose gel electrophoresis to confirm that a ca. 1.4 kb fragment corresponding to the ywfE fragment was amplified. Then, the remaining reaction mixture was mixed with an equal amount of phenol/chloroform saturated with TE. The resulting solution was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained solution (5 μl) was subjected to reaction to cleave the amplified DNA with restriction enzymes NcoI and BamHI. DNA fragments were separated by agarose gel electrophoresis, and a 1.4 kb DNA fragment containing ywfE was recovered using GENECLEAN II Kit.

C-Terminal His-tagged recombinant expression vector pQE60 (Qiagen, Inc.) (0.2 g) was cleaved with restriction enzymes NcoI and BamHI. DNA fragments were separated by agarose gel electrophoresis, and a 3.4 kb DNA fragment was recovered in the same manner as above.

The 1.4 kb DNA fragment containing ywfE and the 3.4 kb DNA fragment obtained above were subjected to ligation reaction using a ligation kit at 16° C. for 16 hours.

Escherichia coli NM522 was transformed using the ligation reaction mixture according to the method using calcium ion, spread on LB agar medium containing 50 μg/ml ampicillin, and cultured overnight at 30° C.

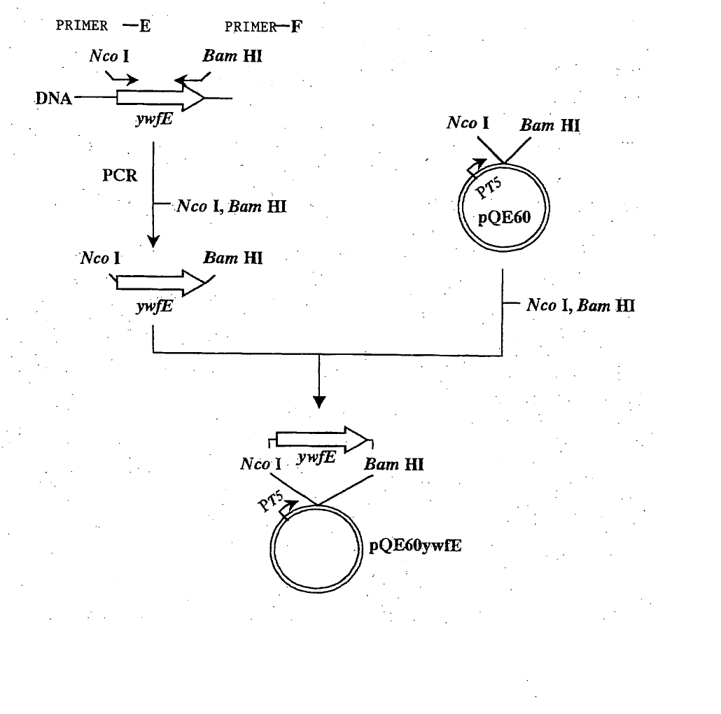

A plasmid was extracted from a colony of the transformant that grew on the medium according to a known method and the structure of the plasmid was analyzed using restriction enzymes, whereby it was confirmed that pQE60ywfE, which is a C-terminal His-tagged ywfE expression vector, was obtained (FIG. 2).

Escherichia coli NM522 carrying pQE60ywfE (Escherichia coli NM522/pQE60ywfE) was inoculated into 8 ml of LB medium containing 50 μg/ml ampicillin in a test tube, and cultured at 28° C. for 17 hours. The resulting culture was inoculated into 50 ml of LB medium containing 50 μg/ml ampicillin in a 250-ml Erlenmeyer flask, and cultured at 30° C. for 3 hours. Then, isopropyl-1-D-thiogalactopyranoside (IPTG) was added to give a final concentration of 1 mmol/l, followed by further culturing at 30° C. for 4 hours. The resulting culture was centrifuged to obtain wet cells, and a His-tagged recombinant enzyme was purified from the wet cells using HisTrap (His-tagged protein purification kit, Amersham Pharmacia Biotech) according to the instructions attached thereto.

### EXPERIMENTAL EXAMPLE 5

### Production of Dipeptides Using the His-Tagged Recombinant Enzyme (1)

(i) A reaction mixture (0.1 ml) comprising 0.04 mg of the purified His-tagged recombinant enzyme obtained in Experimental Example 4, 100 mmol/l Tris-HCl (pH 8.0), 60 mmol/1 magnesium chloride, 60 mmol/l ATP, 30 mmol/l L-Ala and 30 mmol/l L-Gln was prepared, and reaction was carried out at 37° C. for 16 hours.

After the completion of reaction, the reaction product was analyzed in the same manner as in Experimental Example 3 above, whereby it was confirmed that 3.7 g/l L-Ala-L-Gln and 0.3 g/l L-alanyl-L-alanine (L-Ala-L-Ala) were formed and accumulated in the reaction mixture.

(ii) Reactions were carried out under the same conditions as in the above (i) using reaction mixtures having the same composition as that of the reaction mixture of the above (i) except that 0.01 mg of the enzyme was used and L-Phe, L-Met, L-Leu and L-Val, respectively, were used in place of L-Gln.

After the completion of reactions, the reaction products were analyzed in the same manner as in Experimental Example 3 above, whereby it was confirmed that the following dipeptides were formed and accumulated in the respective reaction mixtures: 7.0 g/l L-alanyl-L-phenylalanine (L-Ala-L-Phe) alone; 7.0 g/l L-alanyl-L-methionine (L-Ala-L-Met) and 0.03 g/l L-Ala-L-Ala; 5.0 g/l L-alanyl-L-leucine (L-Ala-L-Leu) and 0.2 g/l L-Ala-L-Ala; and 1.6 g/l L-alanyl-L-valine (L-Ala-L-Val) and 0.3 g/l L-Ala-L-Ala.

(iii) Reactions were carried out under the same conditions as in the above (i) using reaction mixtures having the same composition as that of the reaction mixture of the above (i) except that 0.01 mg of the enzyme was used, Gly was used in place of L-Ala, and L-Phe and L-Met, respectively, were used in place of L-Gln.

After the completion of reactions, the reaction products were analyzed in the same manner as in Experimental Example 3 above, whereby it was confirmed that 5.2 g/l glycyl-L-phenylalanine (Gly-L-Phe) and 1.1 g/l glycyl-L-methionine (Gly-L-Met) were formed and accumulated in the respective reaction mixtures.

When ATP was excluded from the compositions of the above reaction mixtures, no dipeptide was formed.

The above results revealed that the ywfE gene product has the activity to produce, in the presence of ATP, the following dipeptides: L-Ala-L-Gln plus L-Ala-L-Ala, L-Ala-L-Phe, L-Ala-L-Met plus L-Ala-L-Ala, L-Ala-L-Leu plus L-Ala-L-Ala, or L-Ala-L-Val plus L-Ala-L-Ala from L-Ala plus L-Gln, L-Phe, L-Met, L-Leu or L-Val; and Gly-L-Phe or Gly-L-Met from Gly plus L-Phe or L-Met.

### EXPERIMENTAL EXAMPLE 6

### Production of Dipeptides Using the His-Tagged Recombinant Enzyme (2)

A reaction mixture (0.1 ml) comprising 0.04 mg of the purified His-tagged recombinant enzyme obtained in Experimental Example 4, 100 mmol/l Tris-HCl (pH 8.0), 60 mmol/1 magnesium chloride and 60 mmol/l ATP was prepared. To this mixture were respectively added combinations of various L-amino acids, Gly and β-Ala selected from the amino acids shown in the first row of Table 1 and in the leftmost column of Table 1 to give a concentration of 30 mmol/l each, and the resulting mixtures were subjected to reaction at 37° C. for 16 hours. After the completion of reactions, the reaction products were analyzed by HPLC, whereby it was confirmed that the dipeptides shown in Table 1 were formed.

TABLE 1

| | Ala | Gln | Glu | Gly | Val | Leu | Ile | Pro |
|---|---|---|---|---|---|---|---|---|
| Ala | AlaAla | AlaGln | AlaAla | AlaGly | AlaVal | AlaLeu | AlaIle | AlaAla |
| | | AlaAla | | AlaAla | AlaAla | AlaAla | AlaAla | |
| Gln | | X | X | GlyGln | X | X | X | X |
| | | | | GlyGly | | | | |
| Glu | | | | GlyGly | | | | |
| Gly | | | | GlyGly | | | | GlyGly |
| Val | | | | | | | | |
| Leu | | | | | | | | |
| Ile | | | | | | | | |
| Pro | | | | | | | | |
| Phe | | | | | | | | |
| Trp | | | | | | | | |
| Met | | | | | | | | |
| Ser | | | | | | | | |
| Thr | | | | | | | | |
| Cys | | | | | | | | |
| Asn | | | | | | | | |
| Tyr | | | | | | | | |
| Lys | | | | | | | | |
| Arg | | | | | | | | |
| His | | | | | | | | |
| Asp | | | | | | | | |
| αAB | | | | | | | | |
| β-Ala | | | | | | | | |
| Cit | | | | | | | | |
| | Phe | Trp | Met | Ser | Thr | Cys | Asn | Tyr |
| Ala | AlaPhe | AlaTrp | AlaMet | AlaSer | AlaThr | AlaAla | AlaAsn | AlaTyr |
| | AlaAla | AlaAla | | AlaAla | AlaAla | ◯ | AlaAla | AlaAla |
| Gln | ◯ | X | MetMet | SerGln | ThrGln | ◯ | X | X |
| | | | | SerSer | ThrThr | | | |
| Glu | | | | | | | | |
| Gly | GlyPhe | GlyGly | GlyMet | GlySer | GlyThr | GlyGly | GlyGly | GlyTyr |
| | | ◯ | GlyGly | GlyGly | GlyGly | ◯ | | GlyGly |
| | | | | SerGly | ThrGly | | | |
| | | | | SerSer | ThrThr | | | |
| Val | | | X | | | | | |
| Leu | | | MetMet | | ThrLeu | | | |
| Ile | | | MetMet | | | | | |
| Pro | | | MetMet | SerSer | ThrThr | | | |
| Phe | | | MetPhe | SerPhe | ThrPhe | | | |
| | | | MetMet | | ThrThr | | | |
| Trp | | | | | | | | |
| Met | | | MetMet | SerMet | ThrMet | MetMet | | MetTyr |
| | | | | | ThrThr | ◯ | | MetMet |
| Ser | | | | SerSer | SerThr | | | SerTyr |
| | | | | | SerSer | | | SerSer |
| | | | | | ThrSer | | | |
| | | | | | ThrThr | | | |
| Thr | | | | | ThrThr | | | |
| Cys | | | | | | | | |
| Asn | | | | | | | | |
| Tyr | | | | | | | | |
| Lys | | | | | | | | |
| Arg | | | | | | | | |
| His | | | | | | | | |
| Asp | | | | | | | | |
| α-AB | | | | | | | | |
| β-Ala | | | | | | | | |
| Cit | | | | | | | | |

| | Lys | Arg | His | Asp | α-AB | β-Ala | Cit | Azaserine | Theanine |
|---|---|---|---|---|---|---|---|---|---|
| Ala | AlaAla | AlaArg | AlaHis | AlaAla | AlaAla | | AlaAla | AlaAla | AlaAla |
| | ◯ | AlaAla | AlaAla | | ◯ | | ◯ | ◯ | ◯ |
| Gln | X | X | X | X | ◯ | | | | |
| Glu | | | | | | | | | |
| Gly | GlyGly | GlyArg | GlyGly | GlyGly | GlyGly | | ◯ | | |
| | ◯ | GlyGly | | | ◯ | | | | |
| Val | | | | | | | | | |
| Leu | | | | | | | | | |
| Ile | | | | | | | | | |
| Pro | | | | | | | | | |
| Phe | | | | X | | ◯ | | | |
| Trp | | | | | | | | | |
| Met | MetMet | | MetMet | | | ◯ | | | |
| | ◯ | | ◯ | | | | | | |
| Ser | | | SerHis | | SerSer | | | | |
| | | | | | ◯ | | | | |
| Thr | | | | | ThrThr | | | | |
| | | | | | ◯ | | | | |
| Cys | | | | | | | | | |
| Asn | | | | | | | | | |
| Tyr | | | | | | | | | |
| Lys | | | | | | | | | |
| Arg | | | | | ◯ | | | | |
| His | | | | | | β-AlaHis | | | |
| Asp | | | | | | | | | |
| α-AB | | | | | ◯ | | | | |
| β-Ala | | | | | | | | | |
| Cit | | | | | | ◯ | | | |

The dipeptides formed by the reaction using, as substrates, two (or one) kinds of L-amino acids, Gly and β-Ala shown in the first row and the leftmost column of Table 1 are shown in the respective cells of the table. In the table, ◯ means that a dipeptide was formed though its sequence was unidentified; X means that formation of a dipeptide was not confirmed; and a blank means that reaction was not carried out.

### EXPERIMENTAL EXAMPLE 7

### Production of a Dipeptide Using the Strain Expressing the His-Tagged Recombinant Enzyme

Escherichia coli NM522/pQE60ywfE obtained in Experimental Example 4 was inoculated into 8 ml of LB medium containing 50 μg/ml ampicillin in a test tube, and cultured at 28° C. for 17 hours. The resulting culture was inoculated into 50 ml of LB medium containing 50 μg/ml ampicillin in a 250-ml Erlenmeyer flask, and cultured at 30° C. for 3 hours. Then, IPTG was added to give a final concentration of 1 mmol/l, followed by further culturing at 30° C. for 4 hours. The resulting culture was centrifuged to obtain wet cells.

A reaction mixture (20 ml, pH 7.2) comprising 200 g/l wet cells, 50 g/l glucose, 5 g/l phytic acid (diluted to neutrality with 33% conc. sodium hydroxide solution), 15 g/l potassium dihydrogenphosphate, 5 g/l magnesium sulfate heptahydrate, 4 g/l Nymeen S-215, 10 ml/l xylene, 200 mmol/l L-Ala and 200 mmol/l L-Gln was put in a 50-ml beaker, and reaction was carried out at 32° C. at 900 rpm for 2 hours. During the reaction, the pH of the reaction mixture was maintained at 7.2 by using 2 mol/l potassium hydroxide.

The reaction product was analyzed by the same method as in Experimental Example 3, whereby it was confirmed that 25 mg/l L-Ala-L-Gln was accumulated.

### EXPERIMENTAL EXAMPLE 8

Cloning of Genes Corresponding to the ywfE Gene from Various Microorganisms of the Genus Bacillus and Analysis Thereof

On the basis of the nucleotide sequence shown in SEQ ID NO: 9, genes corresponding to the ywfE gene which exist in Bacillus subtilis ATCC 15245, ATCC 6633, IAM 1213, IAM 1107, IAM 1214, ATCC 9466, IAM 1033 and ATCC 21555, Bacillus amyloliquefaciens IFO 3022 and Bacillus pumilus NRRL B-12025 were obtained in the following manner.

That is, Bacillus subtilis ATCC 15245, ATCC 6633, IAM 1213, IAM 1107, IAM 1214, ATCC 9466, IAM 1033 and ATCC 21555, Bacillus amyloliquefaciens IFO 3022 and Bacillus pumilus NRRL B-12025 were respectively inoculated into LB medium and subjected to static culture overnight at 30° C. After the culturing, the chromosomal DNAs of the respective microorganisms were isolated and purified according to the method using saturated phenol described in Current Protocols in Molecular Biology.

By using a DNA synthesizer (Model 8905, PerSeptive Biosystems, Inc.), DNAs having the nucleotide sequences shown in SEQ ID NOS: 25 and 26 (hereinafter referred to as primer G and primer H, respectively) were synthesized. Primer G has a sequence containing a region upstream of the initiation codon of ywfE on the chromosomal DNA of Bacillus subtilis 168, and primer H has a sequence complementary to a sequence containing a region downstream of the termination codon of ywfE.

PCR was carried out using each of the chromosomal DNAs of Bacillus subtilis ATCC 15245, ATCC 6633, IAM 1213, IAM 1107, IAM 1214, ATCC 9466, IAM 1033 and ATCC 21555 and Bacillus amyloliquefaciens IFO 3022 as a template and the above primer G and primer H as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

One-tenth of each of the resulting reaction mixtures was subjected to agarose gel electrophoresis to confirm that a ca. 1.6 kb fragment corresponding to the ywfE fragment was amplified. Then, the remaining reaction mixture was mixed with an equal amount of phenol/chloroform saturated with TE. The resulting solution was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

Each of the thus obtained 1.4 kb DNA fragments derived from the chromosomal DNAs of the respective strains and pCR-blunt (Invitrogen Corp.) were subjected to ligation reaction using a ligation kit at 16° C. for 16 hours.

Escherichia coli NM522 was transformed using each ligation reaction mixture according to the method using calcium ion, spread on LB agar medium containing 50 μg/ml ampicillin, and cultured overnight at 30° C.

A plasmid was extracted from a colony of each transformant that grew on the medium according to a known method and the structure of each plasmid was analyzed using restriction enzymes. As a result, it was confirmed that the following plasmids containing a gene corresponding to the ywfE gene were obtained: pYWFE1 (derived from ATCC 15245, DNA having the nucleotide sequence shown in SEQ ID NO: 36), pYWFE2 (derived from ATCC 6633, DNA having the nucleotide sequence shown in SEQ ID NO: 10), pYWFE3 (derived from IAM 1213, DNA having the nucleotide sequence shown in SEQ ID NO: 11), pYWFE4 (derived from IAM 1107, DNA having the nucleotide sequence shown in SEQ ID NO: 12), pYWFE5 (derived from IAM 1214, DNA having the nucleotide sequence shown in SEQ ID NO: 13), pYWFE6 (derived from ATCC 9466, DNA having the nucleotide sequence shown in SEQ ID NO: 9), pYWFE7 (derived from IAM 1033, DNA having the nucleotide sequence shown in SEQ ID NO: 36), pYWFE8 (derived from ATCC 21555, DNA having the nucleotide sequence shown in SEQ ID NO: 14) and pYWFE9 (derived from IFO 3022, DNA having the nucleotide sequence shown in SEQ ID NO: 15).

On the other hand, a gene corresponding to ywfE derived from Bacillus pumilus NRRL B-12025 (DNA having the nucleotide sequence shown in SEQ ID NO: 16) was obtained in the following manner.

PCR was carried out using the chromosomal DNA of the NRRL B-12025 strain prepared above as a template and DNAs respectively consisting of the nucleotide sequences shown in SEQ ID NOS: 27 and 28 as a set of primers. That is, PCR was carried out by 30 cycles, one cycle consisting of reaction at 98° C. for 5 seconds, reaction at 55° C. for 30 seconds and reaction at 72° C. for one minute, using 50 μl of a reaction mixture comprising 0.1 μg of the chromosomal DNA, 0.5 μmol/l each of the primers, 2.5 units of Z-taq polymerase (Takara Bio Inc.), 5 μl of buffer for Z-taq polymerase (10×) (Takara Bio Inc.) and 200 μmol/l each of dNTPs.

One-tenth of the resulting reaction mixture was subjected to agarose gel electrophoresis to confirm that a ca. 0.8 kb fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained 0.8 kb fragment derived from the chromosomal DNA and pGEM T-easy (Promega Corp.) were subjected to ligation reaction using a ligation kit at 16° C. for 16 hours.

Escherichia coli DH5a was transformed using the reaction mixture according to the method using calcium ion, spread on LB agar medium containing 50 μg/ml ampicillin, and cultured overnight at 30° C.

A plasmid was extracted from the transformant obtained above and the nucleotide sequence of the ca. 0.8 kb DNA insert was determined, whereby a sequence from nucleotides 358 to 1160 in the nucleotide sequence shown in SEQ ID NO: 16 was confirmed.

The above plasmid was cleaved with EcoRI and then subjected to agarose gel electrophoresis to separate a DNA fragment. The DNA fragment was purified using GENECLEAN II Kit, and about 0.5 μg of the purified DNA fragment was DIG-labeled using DIG-High Prime DNA Labeling & Detection Starter Kit I (Roche Diagnostics Corp.) according to the instructions attached thereto.

Southern analysis of the chromosomal DNA of the NRRL B-12025 strain was carried out using the DIG-labeled DNA obtained above.

The chromosomal DNA of the NRRL B-12025 strain was completely digested with BamHI, EcoRI, HindIII, KpnI, PstI, SacI, SalI and SphI, respectively, and subjected to agarose gel electrophoresis to separate DNA fragments, followed by transfer to nylon membrane plus charge (Roche Diagnostics Corp.) according to an ordinary method.

After the DNA fragments were fixed on the nylon membrane by UV irradiation, Southern hybridization was carried out using the above probe DNA and the nylon membrane.

The hybridization was carried out by contacting the nylon membrane with the probe DNA at 65° C. for 16 hours, washing the nylon membrane twice with a solution consisting of 0.1% SDS and 2×SSC at room temperature for 5 minutes, and further washing the membrane twice with a solution consisting of 0.1% SDS and 0.5×SSC at 65° C. for 15 minutes. The other operations and conditions and detection of the hybridized DNA were carried out according to the instructions attached to the above-mentioned DIG-High Prime DNA Labeling & Detection Starter Kit I.

As a result, color development was observed at around 3.5 kbp of the fragments completely digested with HindIII and PstI.

Subsequently, the chromosomal DNA of the NRRL B-12025 strain was completely digested with HindIII and PstI, respectively, and subjected to agarose gel electrophoresis to separate DNA fragments. From the respective restriction enzyme-digested DNAs, 3-4 kbp fragments were purified using GENECLEAN II Kit, followed by autocyclization using a ligation kit.

On the basis of the nucleotide sequence of the 0.8 kb DNA fragment determined above, the nucleotide sequences shown in SEQ ID NOS: 29 and 30 were designed and synthesized, and they were used in PCR using the cyclized DNA obtained above as a template. PCR was carried out by 30 cycles, one cycle consisting of reaction at 98° C. for 5 seconds, reaction at 55° C. for 30 seconds and reaction at 72° C. for 3 minutes and 30 seconds, using 50 μl of a reaction mixture comprising 10 ng of the cyclized DNA, 0.5 μmol/l each of the primers, 2.5 units of pyrobest polymerase (Takara Bio Inc.), 5 μl of buffer for pyrobest polymerase (10×) (Takara Bio Inc.) and 200 μmol/l each of dNTPs.

One-tenth of the resulting reaction mixture was subjected to agarose gel electrophoresis to confirm that a ca. 3.0 kb fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained DNA fragment and Zero Blunt PCR Cloning Kit (Invitrogen Corp.) were subjected to ligation reaction using a ligation kit.

Escherichia coli NM522 was transformed using the reaction mixture according to the method using calcium ion, spread on LB agar medium containing 50 μg/ml ampicillin, and cultured overnight at 30° C.

A plasmid was extracted from a colony of the transformant that grew on the medium according to a known method and the structure of the plasmid was analyzed using restriction enzymes. As a result, it was confirmed that plasmid pYWFE10 (derived from NRRL B-12025, DNA having the nucleotide sequence shown in SEQ ID NO: 16) containing a gene corresponding to the ywfE gene was obtained.

The nucleotide sequences of the genes corresponding to the ywfE gene which are respectively contained in the plasmids pYWFE1 to pYWFE10 obtained above were determined using 373A DNA Sequencer.

The amino acid sequences of the proteins encoded by the genes respectively contained in pYWFE1, pYWFE6 and pYWFE7 were identical with the amino acid sequence of the protein encoded by the ywfE gene, whereas those of the proteins encoded by the genes respectively contained in pYWFE2, pYWFE3, pYWFE4, pYWFE5, pYWFE8, pYWFE9 and pYWFE10 were different from the amino acid sequence of the protein encoded by the ywfE gene.

The amino acid sequences of the proteins encoded by the genes corresponding to the ywfE gene which are contained in pYWFE2, pYWFE3, pYWFE4, pYWFE5, pYWFE8, pYWFE9 and pYWFE10, and pYWFE1 and pYWFE7 are shown in SEQ ID NOS: 2 to 8 and 1, respectively, and the nucleotide sequences of these genes are shown in SEQ ID NOS: 10 to 16 and 36, respectively.

### EXPERIMENTAL EXAMPLE 9

### Purification of C-Terminal His-Tagged Recombinant Dipeptide Synthetase

PCR was carried out using each of the chromosomal DNAs of Bacillus subtilis ATCC 15245, ATCC 6633, IAM 1213, IAM 1107, IAM 1214, ATCC 9466, IAM 1033 and ATCC 21555 and Bacillus amyloliquefaciens IFO 3022 as a template and primer A and primer B described in Experimental Example 2 as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

When the chromosomal DNA of Bacillus pumilus NRRL B-12025 was used as a template, PCR was carried out using DNAs respectively having the nucleotide sequences shown in SEQ ID NOS: 31 and 32 as a set of primers under the same conditions as above.

One-tenth of each of the resulting reaction mixtures was subjected to agarose gel electrophoresis to confirm that a ca. 1.4 kb DNA fragment corresponding to the ywfE fragment was amplified. Then, the remaining reaction mixture was mixed with an equal amount of phenol/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

Each of the thus obtained solutions (5 μl) was subjected to reaction to cleave the amplified DNA with restriction enzymes NcoI and BamHI. DNA fragments were separated by agarose gel electrophoresis, and a 1.4 kb DNA fragment containing a gene corresponding to the ywfE gene was recovered using GENECLEAN II Kit.

Subsequently, 0.2 μg of the C-terminal His-tagged recombinant expression vector pQE60 was cleaved with restriction enzymes NcoI and BamHI. DNA fragments were separated by agarose gel electrophoresis, and a 3.4 kb DNA fragment was recovered in the same manner as above.

Each of the 1.4 kb DNA fragments containing a gene corresponding to the ywfE gene of Bacillus subtilis 168 and the 3.4 kb DNA fragment obtained above were subjected to ligation reaction using a ligation kit at 16° C. for 16 hours. Escherichia coli NM522 was transformed using each ligation reaction mixture according to the method using calcium ion, spread on LB agar medium containing 50 μg/ml ampicillin, and cultured overnight at 30° C.

A plasmid was extracted from a colony of each transformant that grew on the medium according to a known method and the structure of each plasmid was analyzed using restriction enzymes. As a result, it was confirmed that the following C-terminal His-tagged gene expression vectors were obtained: pQE60ywfE1 (a vector containing the gene derived from ATCC 15245), pQE60ywfE2 (a vector containing the gene derived from ATCC 6633), pQE60ywfE3 (a vector containing the gene derived from IAM 1213), pQE60ywfE4 (a vector containing the gene derived from IAM 1107), pQE60ywfE5 (a vector containing the gene derived from IAM 1214), pQE60ywfE6 (a vector containing the gene derived from ATCC 9466), pQE60ywfE7 (a vector containing the gene derived from IAM 1033), pQE60ywfE8 (a vector containing the gene derived from ATCC 21555), pQE60ywfE9 (a vector containing the gene derived from IFO 3022) and pQE60ywfE10 (a vector containing the gene derived from NRRL B-12025).

Escherichia coli NM522/pQE60ywfE1 to NM522/pQE60ywfE10 strains obtained above were respectively inoculated into 8 ml of LB medium containing 50 μg/ml ampicillin in a test tube, and cultured at 28° C. for 17 hours. Each of the resulting cultures was inoculated into 50 ml of LB medium containing 50 μg/ml ampicillin in a 250-ml Erlenmeyer flask, and cultured at 30° C. for 3 hours. Then, IPTG was added to give a final concentration of 1 mmol/l, followed by further culturing at 30° C. for 4 hours. The resulting culture was centrifuged to obtain wet cells, and His-tagged recombinant enzymes were purified from the respective wet cells using HisTrap according to the instructions attached thereto.

### EXPERIMENTAL EXAMPLE 10

### Production of Dipeptides Using Purified Enzymes

Reaction mixtures (0.1 ml each) comprising 0.04 mg of the respective recombinant enzymes obtained in Experimental Example 9, 100 mmol/l Tris HCl (pH 8.0), 60 mmol/1 magnesium chloride, 60 mmol/l ATP, 30 mmol/l L-Ala and 30 mmol/l L-Gln were prepared, and reactions were carried out at 37° C. for 16 hours.

After the completion of reactions, the reaction mixtures were analyzed by the method described in Experimental Example 3, whereby it was confirmed that 3.0 to 3.5 g/l L-Ala-L-Gln and 0.25 to 0.3 g/l L-Ala-L-Ala were formed and accumulated.

When ATP was excluded from the compositions of the above reaction mixtures, L-Ala-L-Gln or L-Ala-L-Ala was not formed at all.

The above results revealed that all of the products of the genes obtained in Experimental Example 8 have the activity to produce L-Ala-L-Gln and L-Ala-L-Ala from L-Ala and L-Gln in the presence of ATP.

### EXPERIMENTAL EXAMPLE 11

### Acquisition of the albC Gene and Its Analogous Gene

The albC gene and its analogous gene were obtained from Streptomyces noursei and Streptomyces albulus based on the nucleotide sequence of the albC gene of Streptomyces noursei [Chemistry & Biol., 9, 1355 (2002)] in the following manner.

Streptomyces noursei IFO15452 and Streptomyces albulus IFO14147 were inoculated into KM73 medium [2 g/l yeast extract (Difco) and 10 g/l soluble starch (Wako Pure Chemical Industries, Ltd.)] containing 1% glycine and KP medium [15 g/l glucose, 10 g/l glycerol, 10 g/l polypeptone (Nihon Pharmaceutical Co., Ltd.), 10 g/1 meat extract (Kyokuto Pharmaceutical Industrial Co., Ltd.) and 4 g/l calcium carbonate)], respectively, and subjected to shaking culture overnight at 28° C. Streptomyces noursei IFO15452 and Streptomyces albulus IFO14147 were distributed by National Institute of Technology and Evaluation (NITE) Biological Resource Center (BRC) (2-5-8, Kazusakamatari, Kisarazu-shi, Chiba 292-0818 Japan).

After the culturing, the chromosomal DNAs of the respective microorganisms were isolated and purified according to the method described in Genetic Manipulation of Streptomyces: a Laboratory Manual: John Innes Foundation.

On the basis of the nucleotide sequence of the albC gene, DNAs having the nucleotide sequences shown in SEQ ID NOS: 41 and 42 (hereinafter referred to as primer J and primer K, respectively) were synthesized by using a DNA synthesizer (Model 8905, PerSeptive Biosystems, Inc.). Primer J has a sequence wherein a sequence containing the NcoI recognition sequence is added to the 5′ end of a region containing the initiation codon of the albC gene on the chromosomal DNA of Streptomyces noursei. Primer K has a sequence wherein a sequence containing the BglII recognition sequence is added to the 5′ end of a sequence complementary to a sequence containing the termination codon of the albC gene.

PCR was carried out using each of the chromosomal DNAs of Streptomyces noursei and Streptomyces albulus as a template and the above primer J and primer K as a set of primers. That is, PCR was carried out by 30 cycles, one cycle consisting of reaction at 94° C. for one minute, reaction at 55° C. for 30 seconds and reaction at 72° C. for one minute, using 50 μl of a reaction mixture comprising 0.14 g of the chromosomal DNA as a template, 0.5 μmol/l each of the primers, 2.5 units of Ex Taq DNA polymerase (Takara Bio Inc.), 5 μl of buffer for Ex Taq DNA polymerase (10×) (Takara Bio Inc.), 200 μmol/l each of dNTPs and 5 μl of dimethyl sulfoxide.

One-tenth of each of the resulting reaction mixtures was subjected to agarose gel electrophoresis to confirm that a ca. 0.7 kb DNA fragment was amplified. Then, the remaining reaction mixture was mixed with an equal amount of phenol/chloroform saturated with TE. The resulting solution was centrifuged, and the obtained upper layer was mixed with a two-fold volume of cold ethanol and allowed to stand at −80° C. for 30 minutes. The resulting solution was centrifuged to precipitate DNA, and the obtained DNA was dissolved in 20 μl of TE.

Each of the thus obtained solutions (5 μl) was subjected to reaction to cleave the amplified DNA with restriction enzymes NcoI and BglII. DNA fragments were separated by agarose gel electrophoresis, and a 700 bp DNA fragment was recovered using GENECLEAN II Kit.

Subsequently, 0.2 μg of the expression vector pQE60 containing phage T5 promoter was cleaved with restriction enzymes NcoI and BglII. DNA fragments were separated by agarose gel electrophoresis, and a 3.4 kb DNA fragment was recovered in the same manner as above.

Each of the actinomycetes-derived 0.7 kb DNA fragments and the pQE60-derived 3.4 kb DNA fragment obtained above were subjected to ligation reaction using a ligation kit at 16° C. for 16 hours.

Escherichia coli NM522 was transformed using each ligation reaction mixture according to the method using calcium ion, spread on LB agar medium containing 50 μg/ml ampicillin, and cultured overnight at 30° C.

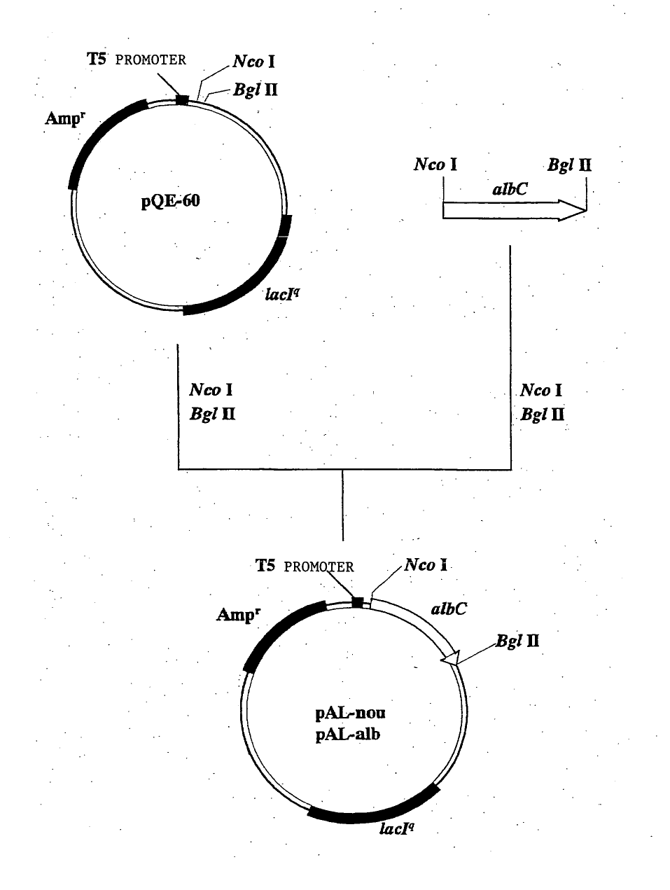

A plasmid was extracted from a colony of each transformant that grew on the medium according to a known method, and the structure of each plasmid was analyzed using restriction enzymes. As a result, it was confirmed that expression vector pAL-nou containing the DNA derived from Streptomyces noursei in a downstream position of the phage T5 promoter and expression vector pAL-alb containing the DNA derived from Streptomyces albulus were obtained (FIG. 3).

The nucleotide sequence of each actinomycete-derived DNA inserted into the respective plasmid was determined by using a nucleotide sequencer (373A DNA Sequencer), whereby it was confirmed that pAL-alb contained DNA encoding a protein having the amino acid sequence shown in SEQ ID NO: 37, i.e. DNA having the nucleotide sequence shown in SEQ ID NO: 39, and pAL-nou contained DNA encoding a protein having the amino acid sequence shown in SEQ ID NO: 38, i.e. DNA having the nucleotide sequence shown in SEQ ID NO: 40.

### EXPERIMENTAL EXAMPLE 12

### Production of Straight-Chain Dipeptides by the Use of Cells as an Enzyme Source

Escherichia coli NM522 carrying pAL-nou or pAL-alb obtained in Experimental Example 11 (Escherichia coli NM522/pAL-nou or NM522/pAL-alb) and Escherichia coli NM522 without a plasmid were respectively inoculated into 10 ml of LB medium containing 50 μg/ml ampicillin in a test tube (no addition of ampicillin in the case of a strain carrying no plasmid, hereinafter the same shall apply), and cultured at 30° C. for 17 hours. Each of the resulting cultures (0.5 ml) was inoculated into 50 ml of LB medium in a 250-ml Erlenmeyer flask and subjected to shaking culture at 30° C. for one hour. Then, IPTG was added to give a final concentration of 1 mmol/l, followed by further culturing for 4 hours. The resulting culture was centrifuged to obtain wet cells.

A reaction mixture (3.0 ml) comprising 100 mg/ml (final concentration) wet cells, 60 mmol/l potassium phosphate buffer (pH 7.2), 10 mmol/1 magnesium chloride, 10 mmol/l ATP, 1 g/l L-Leu and 1 g/l L-Phe was prepared, and reaction was carried out at 30° C. One hour after the start of the reaction, the reaction mixture was sampled and acetonitrile was added thereto to a concentration of 20% (v/v). Then, the obtained reaction product was analyzed by HPLC. The HPLC analysis was carried out by using ODS-HA column (YMC Co., Ltd.) as a separation column and 30% (v/v) acetonitrile as an eluent at a flow rate of 0.6 ml/min, and by measuring ultraviolet absorption at 215 nm.

As a result, it was confirmed that 36.7 mg/l cyclo(L-leucyl-L-phenylalanine) [cyclo(L-Leu-L-Phe)] was accumulated in the reaction mixture of Escherichia coli NM522/pAL-nou. However, no cyclo(L-Leu-L-Phe) was detected in the reaction mixture of Escherichia coli NM522. The same reaction mixtures were analyzed by HPLC under the following conditions to measure linear dipeptides (hereinafter referred to as dipeptides) L-leucyl-L-phenylalanine (L-Leu-L-Phe) and L-phenylalanyl-L-leucine (L-Phe-L-Leu).

Both the dipeptides were derivatized by the F-moc method and then analyzed by HPLC. The HPLC analysis was carried out by using ODS-HG5 (Nomura Kagaku Co., Ltd.) as a separation column and solution A (6 ml/l acetic acid and 20% (v/v) acetonitrile, pH adjusted to 4.8 with triethylamine) and solution B (6 ml/l acetic acid and 70% (v/v) acetonitrile, pH adjusted to 4.8 with triethylamine) as eluents at a flow rate of 0.6 ml/min, and by detecting the dipeptides at an excitation wavelength of 254 nm and a fluorescence wavelength of 630 nm. The ratio of solution A to solution B was 8:2 during the first 5 minutes of elution and thereafter changed with a linear gradient so that the ratio became 1:1 at 20 minutes after the start of elution.

As a result, it was confirmed that 21.9 mg/l L-Leu-L-Phe and 12.0 mg/l L-Phe-L-Leu were accumulated in the reaction mixture of Escherichia coli NM522/pAL-nou and no linear dipeptide was detected in the reaction mixture of Escherichia coli NM522 used as a control strain.

The above result revealed that the cyclodipeptide-synthesizing enzyme obtained in Experimental Example 11 as the ability to synthesize dipeptides.

### EXPERIMENTAL EXAMPLE 13

### Production of Straight-Chain Dipeptides Using the Purified Enzyme (1)

Escherichia coli NM522/pAL-nou was cultured in the same manner as in Experimental Example 12. After the completion of the culturing, centrifugation was carried out to obtain wet cells. The obtained wet cells were washed with a 60 mmol/l potassium phosphate buffer (pH 7.2) and suspended in a 20 mmol/l potassium phosphate buffer containing 10 mmol/l imidazole. The resulting suspension was subjected to ultrasonication at 4° C. to obtain a disrupted cell suspension. The obtained suspension (10 ml: containing 0.863 mg of protein) was passed through a His-tag purification column (Amersham Biosciences K.K.) and then 15 ml of a 20 mmol/l potassium phosphate buffer containing 10 mmol/l imidazole was passed through the column for washing to purify a His-tagged albC protein in the column. Then, 2 ml of a reaction mixture having the same composition as that in Example 2 [composition: 60 mmol/l potassium phosphate buffer (pH 7.2), 10 mmol/1 magnesium chloride, 10 mmol/l ATP, 1 g/l. L-Leu, 1 g/l L-Phe] was put into the column containing the His-tagged albC protein, followed by incubation at 30° C., during which the substrates were held in the column. After 24 hours, the reaction mixture in the column was eluted with 3 ml of a reaction mixture having the same composition, and the cyclodipeptide and linear dipeptides in the reaction mixture were determined in the same manner as in Experimental Example 12.

As a result, it was confirmed that 6.8 mg/l cyclo(L-Leu-L-Phe), 28.7 mg/l L-Leu-L-Phe and 18.5 mg/l L-Phe-L-Leu were formed. No cyclodipeptide or dipeptide was detected in the reaction mixture without ATP incubated in the same manner.

### EXPERIMENTAL EXAMPLE 14

### Production of Dipeptides Using the Purified Enzyme (2)

Enzymatic reaction was carried out in the same manner as in Experimental Example 13 except that the amino acids as substrates were replaced by another amino acid and the obtained product was analyzed. As the reaction mixture, a mixture having the same composition as that of Experimental Example 13 except that the amino acids as the substrates were replaced by 1 g/l L-Ala, L-Leu or L-Phe was used.

As a result, it was revealed that 9.41 mg/l L-Ala-L-Ala, 7.85 mg/l L-Leu-L-Leu and 5.20 mg/l L-Phe-L-Phe were respectively formed in 24 hours after the start of the reaction.

### EXPERIMENTAL EXAMPLE 15

### Construction of Escherichia coli for Enhanced Expression of the ywfE Gene

By using a DNA synthesizer (Model 8905, PerSeptive Biosystems, Inc.), DNAs having the sequences shown in SEQ ID NOS: 82 to 85 (hereinafter referred to as primer L, primer M, primer N and primer 0, respectively) were synthesized. The sequence of SEQ ID NO: 82 is a sequence wherein a sequence containing the XhoI recognition sequence is added to the 5′ end of a region containing the Shine-Dalgarno sequence (ribosome binding sequence) of the ywfE gene on the plasmid pQE60ywfE. The sequence of SEQ ID NO: 83 is a sequence wherein a sequence containing the BamHI recognition sequence is added to the 5′ end of a sequence complementary to a sequence containing the termination codon of the ywfE gene. The sequence of SEQ ID NO: 84 is a sequence wherein a sequence containing the EcoRI recognition sequence is added to the 5′ end of the sequence of trp promoter region of expression vector pTrS30 containing trp promoter. The sequence of SEQ ID NO: 85 is a sequence wherein a sequence containing the XhoI recognition sequence is added to the 5′ end of a sequence complementary to the sequence of trp promoter region of expression vector pTrS30 containing trp promoter.

A ywfE gene fragment and a trp promoter region fragment were amplified by PCR using the above primers L and M and primers N and O as a set of primers, respectively, and the plasmid pQE60ywfE as a template. PCR was carried out by 30 cycles, one cycle consisting of reaction at 94° C. for one minute, reaction at 55° C. for 2 minutes and reaction at 72° C. for 3 minutes, using 40 μl of a reaction mixture comprising 10 ng of pQE60ywfE, 0.5 μmol/l each of the primers, 2.5 units of Pfu DNA polymerase, 4 μl of buffer for Pfu DNA polymerase (10×) and 200 μmol/l each of dNTPs.

One-tenth of each of the resulting reaction mixtures was subjected to agarose gel electrophoresis to confirm that a ca. 1.4 kb fragment corresponding to the ywfE gene fragment and a ca. 0.3 kb fragment corresponding to the trp promoter region fragment were respectively amplified in the PCR using primer L and primer M and the PCR using primer N and primer O. Then, the remaining reaction mixture was mixed with an equal amount of phenol/chloroform saturated with TE. The resulting solution was centrifuged, and the obtained upper layer was mixed with a two-fold volume of cold ethanol and allowed to stand at −80° C. for 30 minutes. The resulting solution was centrifuged, and the obtained DNA was dissolved in 20 μl of TE.

The thus obtained DNA solutions (5 μl each) were respectively subjected to reaction to cleave the DNA amplified using primer L and primer M with restriction enzymes XhoI and BamHI and to reaction to cleave the DNA amplified using primer N and primer 0 with restriction enzymes EcoRI and XhoI. DNA fragments were separated by agarose gel electrophoresis, and a 1.4 kb fragment containing the ywfE gene and a 0.3 kb fragment containing trp promoter region were respectively recovered using GENECLEAN II Kit.

pTrS30 (a trp promoter-containing expression vector, 0.2 μg) was cleaved with restriction enzymes EcoRI and BamHI. DNA fragments were separated by agarose gel electrophoresis and a 4.5 kb DNA fragment was recovered in the same manner as above.

The 1.4 kb fragment containing the ywfE gene, the 0.3 kb fragment containing trp promoter region and the 4.5 kb DNA fragment obtained above were subjected to ligation reaction using a ligation kit at 16° C. for 16 hours.

Escherichia coli NM522 was transformed using the reaction mixture according to the method using calcium ion, spread on LB agar medium containing 50 μg/ml ampicillin, and cultured overnight at 30° C.

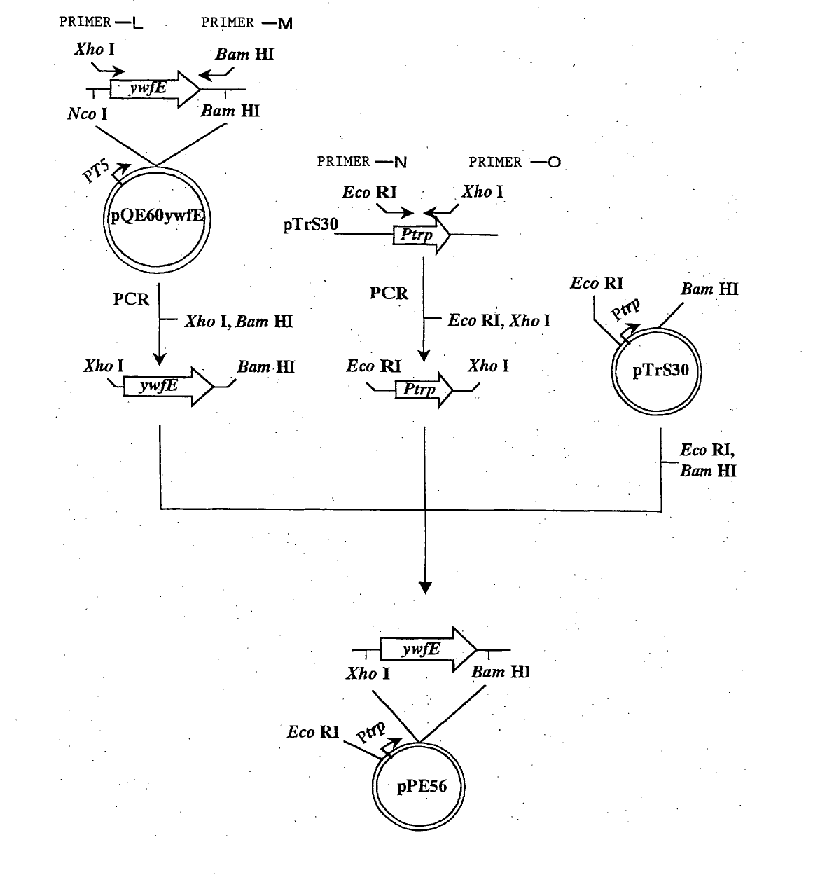

A plasmid was extracted from a colony of the transformant that grew on the medium according to a known method, whereby expression vector pPE56 containing the ywfE gene in a downstream position of the trp promoter was obtained. The structure of the vector was confirmed by digestion with restriction enzymes (FIG. 4).

### EXPERIMENTAL EXAMPLE 16

### Preparation of Strains Having Deletions of the Pepd, Pepn, pepB and pepA Genes and the dpp Operon

Strains in which specific genes on Escherichia coli chromosomal DNA are deleted were prepared according to the method utilizing the homologous recombination system of lambda phage [Proc. Natl. Acad. Sci. USA, 97, 6641-6645 (2000)].

Plasmids pKD46, pKD3 and pCP20 used below were prepared by extraction, according to a known method, from Escherichia coli strains carrying them which were obtained from Escherichia coli Genetic Stock Center, Yale University, U.S.A.

### (1) Cloning of DNA Fragments for Gene Deletion

For the purpose of deleting the following genes existing on the chromosomal DNA of Escherichia coli K12, DNAs having nucleotide sequences homologous to 36-bp nucleotide sequences that lie upstream and downstream of the respective genes to be deleted on the chromosomal DNA of Escherichia coli K12 and the nucleotide sequence shown in SEQ ID NO: 52 which is recognized by yeast-derived Flp recombinase were synthesized using a DNA synthesizer (Model 8905, PerSeptive Biosystems, Inc.). The genes to be deleted are the pepD gene having the nucleotide sequence shown in SEQ ID NO: 53, the pepN gene having the nucleotide sequence shown in SEQ ID NO: 54, the pepB gene having the nucleotide sequence shown in SEQ ID NO: 55, the pepA gene having the nucleotide sequence shown in SEQ ID NO: 56, the dppA gene having the nucleotide sequence shown in SEQ ID NO: 57, the dppB gene having the nucleotide sequence shown in SEQ ID NO: 58, the dppC gene having the nucleotide sequence shown in SEQ ID NO: 59, the dppD gene having the nucleotide sequence shown in SEQ ID NO: 60 and the dppF gene having the nucleotide sequence shown in SEQ ID NO: 61. In the case of the dppA, dppB, dppC, dppD and dppF genes, which form an operon, DNAs having nucleotide sequences homologous to the nucleotide sequences that lie upstream and downstream of the operon were synthesized.

That is, DNAs consisting of the following nucleotide sequences were synthesized as respective sets of primers for amplification of DNA fragments for gene deletion: SEQ ID NOS: 62 and 63 for pepD gene deletion; SEQ ID NOS: 64 and 65 for pepN gene deletion; SEQ ID NOS: 66 and 67 for pepA gene deletion; SEQ ID NOS: 68 and 69 for pepB gene deletion; and SEQ ID NOS: 70 and 71 for dpp operon deletion.

Subsequently, PCR was carried out using each set of the above synthetic DNAs as a set of primers and pKD3 DNA as a template. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 10 ng of the plasmid DNA, 0.5 μmol/l each of the primers, 2.5 units of Pfu DNA polymerase, 4 μl of buffer for Pfu DNA polymerase (10×) and 200 μmol/l each of deoxyNTPs.

One-tenth of each of the resulting reaction mixtures was subjected to agarose gel electrophoresis to confirm that the desired fragment was amplified. Then, the remaining reaction mixture was mixed with an equal amount of phenol/chloroform saturated with TE.

The resulting mixture was centrifuged, and the obtained upper layer was mixed with a two-fold volume of cold ethanol and allowed to stand at −80° C. for 30 minutes, followed by centrifugation. By this procedure, chloramphenicol resistance gene-containing DNA fragments for deletion of the pepD, pepN, pepB and pepA genes and the dpp operon were obtained.

(2) Preparation of Escherichia coli JM101 Having pepD Gene Deletion

Escherichia coli JM101 was transformed with pKD46, spread on LB agar medium containing 100 mg/l ampicillin, and cultured at 30° C. to select Escherichia coli JM101 carrying pKD46 (hereinafter referred to as Escherichia coli JM101/pKD46).

The plasmid pKD46 carries A Red recombinase gene the expression of which can be induced by L-arabinose. Accordingly, when Escherichia coli carrying pKD46 grown in the presence of L-arabinose is transformed using a linear DNA, homologous recombination occurs with high frequency. Further, as pKD46 has a thermosensitive replication origin, curing of the plasmid can be readily caused by culturing the strain at 42° C.

The chloramphenicol resistance gene-containing DNA fragment for pepD gene deletion obtained above was introduced into Escherichia coli JM101/pKD46 obtained by culturing in the presence of 10 mmol/l L-arabinose and 50 μg/ml ampicillin by electroporation. The resulting cells were spread on LB agar medium (10 g/l Bacto-tryptone, 5 g/l Bacto-yeast extract, 5 g/l sodium chloride and 15 g/l agar) containing 25 mg/l chloramphenicol and cultured at 30° C. to select a transformant in which the chloramphenicol resistance gene-containing DNA fragment for pepD gene deletion was integrated into the chromosomal DNA of Escherichia coli JM101 by homologous recombination.

The selected chloramphenicol-resistant strain was inoculated onto LB agar medium containing 25 mg/l chloramphenicol and cultured at 42° C. for 14 hours, followed by single colony isolation. Replicas of the obtained colonies were made on LB agar medium containing 25 mg/l chloramphenicol and LB agar medium containing 100 mg/l ampicillin, followed by culturing at 37° C. By selecting a colony showing chloramphenicol resistance and ampicillin sensitivity, a pKD46-cured strain was obtained.

The pKD46-cured strain thus obtained was transformed using pCP20, followed by selection on LB agar medium containing 100 mg/l ampicillin to obtain a pKD46-cured strain carrying pCP20.

The plasmid pCP20 carries yeast-derived Flp recombinase gene the expression of which can be induced at a temperature of 42° C.

The chloramphenicol resistance gene-containing DNA fragments for deletion of the pepD, pepN, pepB and pepA genes and the dpp operon prepared above contain nucleotide sequences recognized by Flp recombinase at both termini of the chloramphenicol resistance gene. Therefore, the resistance gene can be readily deleted by homologous recombination catalyzed by Flp recombinase.

Further, as pCP20 has a thermosensitive replication origin, expression of Flp recombinase and curing of pCP20 can be simultaneously induced by culturing the pCP20-carrying strain at 42° C.

The pCP20-carrying pKD46-cured strain obtained above was inoculated onto drug-free LB agar medium and cultured at 42° C. for 14 hours, followed by single colony isolation. Replicas of the obtained colonies were made on drug-free LB agar medium, LB agar medium containing 25 mg/l chloramphenicol and LB agar medium containing 100 mg/l ampicillin, followed by culturing at 30° C. Then, colonies showing chloramphenicol sensitivity and ampicillin sensitivity were selected.

Chromosomal DNAs were prepared from the respective strains selected above according to an ordinary method [Seibutsukogaku Jikkensho (Experiments in Biotechnology), edited by The Society for Biotechnology, Japan, p. 97-98, Baifukan (1992)]. PCR was carried out using, as a set of primers, DNAs having the nucleotide sequences shown in SEQ ID NOS: 72 and 73 which were designed based on an inner nucleotide sequence of the pepD gene to be deleted, and using each of the chromosomal DNAs as a template. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of deoxyNTPs.

A strain with which no amplified DNA fragment was detected in the above PCR was identified as a strain having pepD gene deletion and was designated as Escherichia coli JPD1.

(3) Preparation of a Strain in which the pepD and pepN
Genes on the Chromosomal DNA of Escherichia coli JM101 are Deleted

Escherichia coli JPD1 obtained in the above (2) was transformed with pKD46, spread on LB agar medium containing 100 mg/l ampicillin, and cultured at 30° C. to select Escherichia coli JPD1 carrying pKD46 (hereinafter referred to as Escherichia coli JPD1/pKD46). The chloramphenicol resistance gene-containing DNA fragment for pepN gene deletion was introduced into Escherichia coli JPD1/pKD46 by electroporation to obtain a transformant in which the chloramphenicol resistance gene-containing DNA fragment for pepN gene deletion was integrated into the chromosomal DNA of Escherichia coli JPD1/pKD46 by homologous recombination.

Subsequently, the same procedure as in the above (2) was carried out to obtain a strain in which the chloramphenicol resistance gene was deleted from the chromosomal DNA, which was designated as Escherichia coli JPDN2.

(4) Preparation of Strains in which the pepN, pepA or pepB Gene or the dpp Operon on the Chromosomal DNA of Escherichia coli JM101 is Deleted and Strains Having Multiple Gene Deletion

The strains having pepN, pepA or pepB gene or dpp operon deletion were prepared according to the same procedure as in the above (2) using the respective chloramphenicol resistance gene-containing DNA fragments for gene or operon deletion prepared in the above (1).

Acquisition of the strains having gene deletions by the above method was confirmed by carrying out PCR in the same manner as in the above (2) using, as sets of primers, DNAs having the nucleotide sequences shown in SEQ ID NOS: 74 to 81 which were designed and syntheseized based on inner nucleotide sequences of the respective genes to be deleted. That is, DNAs having the following nucleotide sequences were used as respective sets of primers for the confirmation of gene deletion: SEQ ID NOS: 74 and 75 for pepN deletion; SEQ ID NOS: 76 and 77 for pepA deletion; SEQ ID NOS.: 78 and 79 for pepB deletion; and SEQ ID NOS: 80 and 81 for dpp operon deletion.

The thus obtained dpp operon-deleted strain, pepN gene-deleted strain, pepA gene-deleted strain and pepB gene-deleted strain were designated as Escherichia coli JDPP1, Escherichia coli JPN1, Escherichia coli JPA1 and Escherichia coli JPB7, respectively.

Further, strains having multiple gene deletions, i.e., deletions of two or more genes or operon selected from the group consisting of the pepD, pepN, pepA and pepB genes and the dpp operon were prepared according to the method of the above (3). Acquisition of the strains having multiple gene deletions was confirmed by PCR similar to that in the above (2). The thus obtained double gene-deleted strain having pepD gene and dpp operon deletions was designated as Escherichia coli JPDP49, triple gene-deleted strain having pepB, pepD and pepN gene deletions as Escherichia coli JPDNB43, triple gene-deleted strain having pepD and pepN gene and dpp operon deletions as Escherichia coli JPNDDP36, quadruple gene-deleted strain having pepA, pepD and pepN gene and dpp operon deletions as Escherichia coli JPNDAP5, and quadruple gene-deleted strain having pepB, pepD and pepN gene and dpp operon deletions as Escherichia coli JPNDBP7. The genes deleted in the gene-deleted strains are shown in Table 2.

TABLE 2

| Strain | Deleted gene |
|---|---|
| JM101 | none |
| JDPP1 | dpp operon |
| JPN1 | pepN |
| JPA1 | pepA |
| JPB7 | pepB |
| JPD1 | pepD |
| JPDN2 | pepD, pepN |
| JPNDB43 | pepB, pepD, pepN |
| JPDP49 | pepD, dpp operon |
| JPNDDP36 | pepD, pepN, dpp operon |
| JPNDAP5 | pepA, pepD, pepN, dpp operon |
| JPNDBP7 | pepB, pepD, pepN, dpp operon |

### EXPERIMENTAL EXAMPLE 17

### Evaluation of Productivity of L-Ala-L-Gln and L-Ala-L-Ala by Escherichia coli Strains in which Peptidase and Dipeptide-Permeating/Transporting Protein Activities are Lost

The strains having deletions of genes encoding various peptidases and dipeptide-permeating/transporting protein which were obtained in Experimental Example 16 were transformed using the plasmid pPE56 constructed in Experimental Example 15 to obtain ampicillin-resistant transformants.

Each of the obtained transformants was inoculated into 8 ml of LB medium containing 50 μg/ml ampicillin in a test tube and cultured at 28° C. for 17 hours. The resulting culture was added to 8 ml of an aqueous medium [16 g/l dipotassium hydrogenphosphate, 14 g/l potassium dihydrogenphosphate, 5 g/l ammonium sulfate, 1 g/l citric acid (anhydrous), 0.5 g/l Casamino acid (Difco), 1 g/l L-Pro, 2.5 g/l L-Ala, 2.5 g/l L-Gln, 10 g/l glucose, 10 mg/l vitamin B₁, 25 mg/l magnesium sulfate heptahydrate and 50 mg/l ferrous sulfate heptahydrate; pH adjusted to 7.2 with 10 mol/l sodium hydroxide solution; L-Gln was added after sterilization by filtration of a 10-fold conc. solution; glucose, vitamin B₁, magnesium sulfate heptahydrate and ferrous sulfate heptahydrate were added after separate steam sterilization] containing 100 μg/ml ampicillin and amino acids in a test tube in an amount of 1% and subjected to reaction at 30° C. for 24 hours. The resulting aqueous medium was centrifuged to obtain a supernatant.

The product in the supernatant was derivatized by the F-moc method and then analyzed by HPLC. The HPLC analysis was carried out using ODS-HG5 (Nomura Kagaku Co., Ltd.) as a separation column and solution A (6 ml/l acetic acid and 20% (v/v) acetonitrile, pH adjusted to 4.8 with triethylamine) and solution B (6 ml/l acetic acid and 70% (v/v) acetonitrile, pH adjusted to 4.8 with triethylamine) as eluents. The ratio of solution A to solution B was 8:2 during the first 5 minutes of elution and thereafter changed with a linear gradient so that the ratio became 1:1 at 20 minutes after the start of elution. The results of analysis are shown in Table 3.

TABLE 3

| | | Ala-Gln | Ala-Ala |
|---|---|---|---|
| Strain | Deleted gene | (g/l) | (g/l) |
| JM101 | none | 0 | 0 |
| JDPP1 | dpp operon | 0.02 | 0.01 |
| JPN1 | pepN | 0.01 | 0.01 |
| JPA1 | pepA | 0.01 | 0.01 |
| JPB7 | pepB | 0.01 | 0.01 |
| JPD1 | pepD | 0.01 | 0.01 |
| JPDN2 | pepD, pepN | 0.02 | 0.03 |
| JPNDB43 | pepB, pepD, pepN | 0.05 | 0.12 |
| JPDP49 | pepD, dpp operon | 0.11 | 0.08 |
| JPNDDP36 | pepD, pepN, dpp operon | 0.16 | 0.21 |
| JPNDAP5 | pepA, pepD, pepN, dpp operon | 0.28 | 0.26 |
| JPNDBP7 | pepB, pepD, pepN, dpp operon | 0.43 | 0.22 |

As can be seen from Table 3, small amounts of dipeptides were formed and accumulated by use of the microorganisms having deletions of two or less kinds of peptidase genes or one operon encoding a peptide-permeating/transporting protein, whereas the amounts of dipeptides formed and accumulated were greatly increased by use of the microorganisms having deletions of one or more kinds of peptidase genes and one operon encoding a peptide-permeating/transporting protein or microorganisms having deletions of three or more kinds of peptidase genes.

### EXPERIMENTAL EXAMPLE 18

### Evaluation of Productivity of L-Alanyl-L-valine (Hereinafter Referred to as L-Ala-L-Val) by Escherichia coli Strains in which Peptidase and Peptide-Permeating/Transporting Protein Activities are Lost

Similarly to Experimental Example 17, the Escherichia coli strains having deletions of genes encoding various peptidases and peptide-permeating/transporting protein were transformed using pPE56. Each of the obtained, transformants was inoculated into 8 ml of LB medium containing 50 μg/ml ampicillin in a test tube and cultured at 28° C. for 17 hours. The resulting culture was added to 8 ml of an aqueous medium [16 g/l dipotassium hydrogenphosphate, 14 g/l potassium dihydrogenphosphate, 5 g/l ammonium sulfate, 1 g/l citric acid (anhydrous), 0.5 g/l Casamino acid (Difco), 1 g/l L-Pro, 2.5 g/l L-Ala, 2.5 g/l L-Val, 10 g/l glucose, 10 mg/l vitamin B₁, 25 mg/l magnesium sulfate heptahydrate and 50 mg/l ferrous sulfate heptahydrate; pH adjusted to 7.2 with 10 mol/l sodium hydroxide solution; glucose, vitamin B₁, magnesium sulfate heptahydrate and ferrous sulfate heptahydrate were added after separate steam sterilization] containing 100 μg/ml ampicillin and amino acids in a test tube in an amount of 1% and subjected to reaction at 30° C. for 24 hours. The resulting aqueous medium was centrifuged to obtain a supernatant.

The product in the culture supernatant was analyzed by the method described in Experimental Example 17. The results are shown in Table 4.

TABLE 4

| | | Ala-Val |
|---|---|---|
| Strain | Deleted gene | (g/l) |
| JM101 | none | 0 |
| JDPP1 | dpp operon | 0 |
| JPN1 | pepN | 0 |
| JPA1 | pepA | 0 |
| JPB7 | pepB | 0 |
| JPD1 | pepD | 0 |
| JPDN2 | pepD, pepN | 0 |
| JPNDB43 | pepB, pepD, pepN | 0.04 |
| JPDP49 | pepD, dpp operon | 0.11 |
| JPNDDP36 | pepD, pepN, dpp operon | 0.22 |
| JPNDBP7 | pepB, pepD, pepN, dpp operon | 0.20 |

As can be seen from Table 4, the dipeptide was not produced by use of the microorganisms having deletions of two or less kinds of peptidase genes or one operon encoding a peptide-permeating/transporting protein, whereas the dipeptide was produced by use of the microorganisms having deletions of three or more kinds of peptidase genes or microorganisms having deletions of one or more kinds of peptidase genes and one operon encoding a peptide-permeating/transporting protein.

### EXPERIMENTAL EXAMPLE 19

### Evaluation of Productivity of Glycyl-L-glutamine (Hereinafter Referred to as Gly-L-Gln) by Escherichia coli Strains in which Peptidase and Dipeptide-Permeating/Transporting Protein Activities are Lost

Similarly to Experimental Example 17, the strains having deletions of various peptidase genes and an operon encoding a dipeptide-permeating/transporting protein were transformed using pPE56. Each of the obtained transformants was inoculated into 8 ml of LB medium containing 50 μg/ml ampicillin in a test tube and cultured at 28° C. for 17 hours.

The resulting culture was added to 8 ml of an aqueous medium [16 g/l dipotassium hydrogenphosphate, 14 g/l potassium dihydrogenphosphate, 5 g/l ammonium sulfate, 1 g/l citric acid (anhydrous), 0.5 g/l Casamino acid (Difco), 1 g/l L-Pro, 2.5 g/l Gly, 2.5 g/l L-Gln, 10 g/l glucose, 10 mg/l vitamin B₁, 25 mg/l magnesium sulfate heptahydrate and 50 mg/l ferrous sulfate heptahydrate; pH adjusted to 7.2 with 10 mol/l sodium hydroxide solution;

L-Gln was added after sterilization by filtration of a 10-fold conc. solution; glucose, vitamin B₁, magnesium sulfate heptahydrate and ferrous sulfate heptahydrate were added after separate steam sterilization] containing 100 μg/ml ampicillin and amino acids in a test tube in an amount of 1% and subjected to reaction at 30° C. for 24 hours. The resulting aqueous medium was centrifuged to obtain a supernatant.

The product in the culture supernatant was analyzed by the method described in Experimental Example 17. The results are shown in Table 5.

TABLE 5

| | | Gly-Gln |
|---|---|---|
| Strain | Deleted gene | (g/l) |
| JM101 | none | 0 |
| JDPP1 | dpp operon | 0 |
| JPDN2 | pepD, pepN | 0 |
| JPNDB43 | pepB, pepD, pepN | 0.01 |
| JPNDDP36 | pepD, pepN, dpp operon | 0.02 |
| JPNDBP7 | pepB, pepD, pepN, dpp operon | 0.03 |

As can be seen from Table 5, the dipeptide was not produced by use of the microorganisms having deletions of two or less kinds of peptidase genes or one operon encoding a peptide-permeating/transporting protein, whereas the dipeptide was produced by use of the microorganisms having deletions of three or more kinds of peptidase genes or microorganisms having deletions of two or more kinds of peptidase genes and one operon encoding a peptide-permeating/transporting protein.

Certain embodiments of the present invention are illustrated in the following examples. These examples are not to be construed as limiting the scope of the invention.

### EXAMPLE 1

### Enzymatic Process for Production of Dipeptides or Dipeptide Derivatives Using Amino Acids or Amino Acid Derivatives as Substrates

A reaction mixture (0.1 ml) comprising 40 mg/l purified His-tagged recombinant enzyme obtained in Experimental Example 4, 100 mmol/l Tris HCl (pH 9.0), 30 mmol/1 magnesium chloride, 10 mmol/l ATP and 10 mmol/l each of amino acid and amino acid derivative shown in Table 6 was prepared, and reaction was carried out at 37° C. for 2 hours.

After the completion of reaction, a termination buffer (4 mol/l urea, 100 mmol/l EDTA disodium salt) (100 times amount of the reaction mixture) was added to the reaction mixture to terminate the reaction, and the amount of ADP formed when ATP was consumed by enzymatic reaction was determined by HPLC, whereby it was confirmed that the reaction proceeded. The HPLC analysis was carried out by using Develosil C30-UG-5 (150×4.6 mm, Nomura Kagaku Co., Ltd.) as a column and a solution comprising 200 mmol/l acetic acid and 200 mmol/l triethylamine (pH 6.6) as a mobile phase at a flow rate of 1.0 ml/min at room temperature, and by measuring ultraviolet absorption at 254 nm. The results are shown in Table 6.

TABLE 6

| | Amount of ADP formed |
|---|---|
| Amino acid added | (mmol/l) |
| none | 0.05 |
| Ala | 0.13 |
| Ala + cyc(5)Ala | 5.67 |
| Ala + cyc(3)Ala | 3.64 |
| Ala + cyc(6)Ala | 3.78 |
| none | 0.05 |
| Ala | 0.13 |
| Phe | 0.02 |
| Ala + Phe | 5.84 |
| Ala + Cl-Phe | 4.78 |
| Cl-Ala + Phe | 2.75 |
| CN-Ala + Phe | 1.11 |
| Ala + F-Phe | 4.44 |
| Ala + Ni-Phe | 4.42 |
| Ala + NH₂-Phe | 1.89 |
| Ala + Kynurenin | 2.11 |
| none | 0.05 |
| Ala | 0.13 |
| Glu | 0.13 |
| Ala + Glu(OMe) | 2.60 |
| Ala + Glu(OEt) | 3.93 |
| Ala + Glu(OtBu) | 6.43 |
| Ala + Glu(OBzl) | 6.01 |
| none | 0.05 |
| Ala | 0.13 |
| Asp | 0.16 |
| Ala + Asp(OMe) | 0.54 |
| Ala + Asp(OtBu) | 4.42 |
| Ala + Asp(OBzl) | 3.23 |
| none | 0.05 |
| Ala | 0.13 |
| Lys | 0.17 |
| Ala + Lys(Ac) | 0.75 |
| Ala + Lys(Boc) | 3.64 |

Tables 6-1 to 6-5 respectively show the results of experiments using the following substrates: 6-1, L-Ala and L-Ala derivatives; 6-2, L-Ala and L-Phe derivatives; 6-3, L-Ala and L-Glu derivatives; 6-4, L-Ala and L-Asp derivatives; and 6-5, L-Ala and L-Lys derivatives. The abbreviations for the amino acids and amino acid derivatives used as the substrates are as follows.

### Ala: L-alanine

### Phe: L-phenylalanine

Glu: L-glutamic acid
Asp: L-aspartic acid

### Lys: L-lysine

Cl-Ala: β-chloro-L-alanine
CN-Ala: β-cyano-L-alanine
cyc(5)Ala: β-cyclopentyl-DL-alanine
cyc(3)Ala: β-cyclopropylalanine
cyc(6)Ala: β-cyclohexylalanine
Cl-Phe: 4-chlorophenylalanine
F-Phe: 4-fluorophenylalanine
Ni-Phe: p-nitrophenylalanine
NH₂-Phe: p-aminophenylalanine
Phe-NH₂: phenylalanine amide

### Kynurenin: L-kynurenin

Glu(OMe): glutamic acid-γ-methyl ester
Glu(OEt): glutamic acid-γ-ethyl ester
Glu(OtBu): glutamic acid-γ-t-butyl ester
Glu(OBzl): glutamic acid-γ-benzyl ester
Asp(OMe): aspartic acid-β-methyl ester
Asp(OtBu): aspartic acid-β-t-butyl ester
Asp(OBzl): aspartic acid-β-benzyl ester
Lys(Ac): acetyl-lysine

### Lys(Boc): Boc-lysine

As shown in Table 6, the amounts of ADP formed in the control experiment using no substrate and the experiments respectively using L-Ala, L-Phe, L-Glu, L-Asp and L-Lys as the sole substrate were 0.02 to 0.16 mmol/l. On the other hand, as much as 0.54 to 6.43 mmol/l ADP was formed by using the combinations of amino acids and amino acid derivatives shown in Table 1.

The structural analysis of compounds that existed in the reaction mixtures obtained under reaction conditions similar to those as mentioned above was carried out by proton NMR analysis. The concentration of an amino acid or an amino acid derivative used as the substrate was 20 mmol/L in reaction mixtures 1, 4, 10 and 11, and 10 mmol/L in the remaining reaction mixtures, respectively.

The proton NMR analysis was carried out by using DMX500 manufactured by Bruker Co. under the conditions described below.

### Temperature; 303K

Standard compound; 1 mmol/L 3-(Trimethylsilyl)-Propionic acid-D4 sodium salt (TSP)
Medium; light water (in the case of measuring reaction mixtures 4, 10 and 11) or heavy water (in the case of measuring the other reaction mixtures)

The structure of each of the compounds in the reaction mixtures was identified based on the Chemical shift of the proton of the α position. As the area of TSP was assumed to be an internal standard, the concentration of each of the compounds was calculated based on the area of the signal of the proton of the α position (Table 7). However, because the concentration of L-Ala-L-Ala was low, and because the signal of the proton of the α position overlapped with other signals, the concentration of L-Ala-L-Ala was calculated based on the area of the signal of the proton of the β position. The Chemical shift of the proton of the α position of each of the compounds are shown in parentheses (the unit is ppm).

The reaction mixture using Cl-Ala and Phe as substrates (Reaction mixture 1)
Cl-Ala(4.20), Phe(4.02), Cl-Ala-Phe(3.93, 4.50), Aziridine-2-carboxylic acid [Azc](2.73), Azc-Phe(2.59, 4.47)
The reaction mixture using CN-Ala and Phe as substrates (Reaction mixture 2)

### CN-Ala(3.87), Phe(4.00), CN-Ala-Phe(3.71, 4.48)

The reaction mixture using Ala and Cl-Phe as substrates (Reaction Mixture 3)

### Ala(3.79), Cl-Phe(3.97), Ala-Cl-Phe(3.90, 4.43)

The reaction mixture using Ala and NH₂-Phe as substrates (Reaction mixture 4)

### Ala(3.78), NH₂-Phe(3.93), Ala-NH₂-Phe(3.95, 4.39), Ala-Ala(B; 1.55, 1.36)

The reaction mixture using Ala and Kinurenine as substrates (Reaction mixture 5)

### Ala(3.79), Kinurenine(4.16), Ala-Kinurenine(3.96, 4.64)

The reaction mixture using Ala and Phe-NH₂ as substrates (Reaction mixture 6)

### Ala(3.79), Phe-NH₂ (4.02), Ala-Phe-NH₂ (3.90, 4.60)

The reaction mixture using Ala and Glu(OMe) as substrates (Reaction mixture 7)

### Ala(3.79), Glu(OMe)(3.76), Ala-Glu(OMe)(4.05, 4.18), Ala-Ala(B; 1.55, 1.36)

The reaction mixture using Ala and Glu(OtBu) as substrates (Reaction mixture 8)

### Ala(3.79), Glu(OtBu)(3.76), Ala-Glu(OtBu)(4.04, 4.18)

The reaction mixture using Ala and Asp(OtBu) as substrates (Reaction mixture 9)

### Ala(3.81), Asp(OtBu)(3.98), Ala-Asp(OtBu)(4.04, 4.46)

The reaction mixture using Ala and Lys(Boc) as substrates (Reaction mixture 10)

### Ala(3.78), Lys(Boc)(3.73), Ala-Lys(Boc)(4.02, 4.14)

The reaction mixture using Ala and cyc(3)Ala as substrates (Reaction mixture 11)
Ala(3.78), cyc(3)Ala (3.82), Ala-cyc(3)Ala(4.08, 4.24)
The reaction mixture using Ala and cyc(6)Ala as substrates (Reaction mixture 12)
Ala(3.79), cyc(6)Ala (3.76), Ala-cyc(6)Ala(4.02, 4.22)

TABLE 7

| Reaction | | |
|---|---|---|
| mixture No. | dipeptide | conc.(mmol/L) |
| 1 | Cl-Ala-Phe | 3.5 |
| | Azc-Phe | 5.3 |
| 2 | Cl-Ala | 8.4 |
| | (Cl-Ala)X2 | overlap |
| 3 | Ala-Cl-Phe | 4.3 |
| 4 | Ala-NH₂-Phe | 10.6 |
| | Ala-Ala | 0.3 |
| 5 | Ala-Kinurenine | 4.0 |
| 6 | Ala-Phe-NH₂ | 9.7 |
| 7 | Ala-Glu(OMe) | 6.5 |
| | Ala-Ala | 0.5 |
| 8 | Ala-Glu(OtBu) | 9.7 |
| 9 | Ala-Asp(OtBu) | 10.6 |
| 10 | Ala-Lys(Boc) | 6.6 |
| 11 | Ala-Cyc(3)Ala | 10.5 |
| 12 | Ala-Cyc(6)Ala | 6.6 |

The term “overlap” in the table means that an accurately fixed quantity could not be determined because the signals were overlapped.

It was thus revealed that a dipeptide derivative in which an amino acid and an amino acid derivative are directly linked by peptide bond can be produced using the amino acid and amino acid derivative as substrates according to the process of the present invention.

### EXAMPLE 2

### Production of N-[2-(Acetylamino)propionyl]phenylalanine

L-Ala-L-Phe obtained in Experimental Example 6 (100 mg, 0.423 mmol) is suspended in methylene chloride (10 ml), and pyridine (10 ml) and acetic anhydride (1 ml, 11 mmol) are added to the suspension at room temperature. After stirring at room temperature for 24 hours, water is added and the resulting mixture is extracted three times with chloroform. The organic layer is washed with a saturated aqueous solution of sodium chloride and dried over anhydrous magnesium sulfate, and then the solvent is distilled away under reduced pressure to obtain N-[2-(acetylamino)propionyl]phenylalanine.

### EXAMPLE 3

### Production of 1-{2-[N-((Acetylamino)acetyl)amino]-3-phenylpropionyl}piperidine

N-[2-(Acetylamino)propionyl]phenylalanine obtained in Example 2 (10 mg, 0.036 mmol) is suspended in N,N-dimethylformamide (5 ml), and 1-(3-dimethylaminopropyl)-3-ethylcarbodiimide hydrochloride (14 mg, 0.073 mmol), 1-hydroxybenzotriazole (15 mg, 0.11 mmol) and piperidine (40 μl, 0.40 mmol) are added to the suspension at room temperature, followed by stirring at 50° C. for 24 hours. To the reaction mixture is added water, and the resulting mixture is extracted three times with chloroform. The organic layer is washed with 10% hydrochloric acid and a saturated aqueous solution of sodium chloride, and dried over anhydrous magnesium sulfate. After the solvent is distilled away under reduced pressure, the residue is purified by silica gel column chromatography to obtain 1-{2-[N-(2-(acetylamino)propionyl)amino]-3-phenylpropionyl}piperidine.

### SEQUENCE LISTING FREE TEXT

### SEQ ID NO: 19—Description of Artificial Sequence: Synthetic DNA

### SEQ ID NO: 20—Description of Artificial Sequence: Synthetic DNA

### SEQ ID NO: 21—Description of Artificial Sequence: Synthetic DNA

### SEQ ID NO: 22—Description of Artificial Sequence: Synthetic DNA

### SEQ ID NO: 23—Description of Artificial Sequence: Synthetic DNA

### SEQ ID NO: 24—Description of Artificial Sequence: Synthetic DNA

### SEQ ID NO: 25—Description of Artificial Sequence: Synthetic DNA

### SEQ ID NO: 26—Description of Artificial Sequence: Synthetic DNA

### SEQ ID NO: 27—Description of Artificial Sequence: Synthetic DNA

### SEQ ID NO: 28—Description of Artificial Sequence: Synthetic DNA

### SEQ ID NO: 29—Description of Artificial Sequence: Synthetic DNA

### SEQ ID NO: 30—Description of Artificial Sequence: Synthetic DNA

### SEQ ID NO: 31—Description of Artificial Sequence: Synthetic DNA

### SEQ ID NO: 32—Description of Artificial Sequence: Synthetic DNA

SEQ ID NO: 33—Description of Artificial Sequence: Amino acid sequence used for database search
SEQ ID NO: 34—Description of Artificial Sequence: Amino acid sequence used for database search
SEQ ID NO: 35—Description of Artificial Sequence: Amino acid sequence used for database search

### SEQ ID NO: 41—Description of Artificial Sequence: Synthetic DNA

### SEQ ID NO: 42—Description of Artificial Sequence: Synthetic DNA

### SEQ ID NO: 52—Description of Artificial Sequence: Synthetic DNA

### SEQ ID NO: 53—Description of Artificial Sequence: Synthetic DNA

### SEQ ID NO: 54—Description of Artificial Sequence: Synthetic DNA

### SEQ ID NO: 55—Description of Artificial Sequence: Synthetic DNA

### SEQ ID NO: 56—Description of Artificial Sequence: Synthetic DNA

### SEQ ID NO: 57—Description of Artificial Sequence: Synthetic DNA

### SEQ ID NO: 58—Description of Artificial Sequence: Synthetic DNA

### SEQ ID NO: 59—Description of Artificial Sequence: Synthetic DNA

### SEQ ID NO: 60—Description of Artificial Sequence: Synthetic DNA

### SEQ ID NO: 61—Description of Artificial Sequence: Synthetic DNA

### SEQ ID NO: 62—Description of Artificial Sequence: Synthetic DNA

### SEQ ID NO: 63—Description of Artificial Sequence: Synthetic DNA

### SEQ ID NO: 64—Description of Artificial Sequence: Synthetic DNA

### SEQ ID NO: 65—Description of Artificial Sequence: Synthetic DNA

### SEQ ID NO: 66—Description of Artificial Sequence: Synthetic DNA

### SEQ ID NO: 67—Description of Artificial Sequence: Synthetic DNA

### SEQ ID NO: 68—Description of Artificial Sequence: Synthetic DNA

### SEQ ID NO: 69—Description of Artificial Sequence: Synthetic DNA

### SEQ ID NO: 70—Description of Artificial Sequence: Synthetic DNA

### SEQ ID NO: 71—Description of Artificial Sequence: Synthetic DNA

### SEQ ID NO: 72—Description of Artificial Sequence: Synthetic DNA

### SEQ ID NO: 73—Description of Artificial Sequence: Synthetic DNA

### SEQ ID NO: 74—Description of Artificial Sequence: Synthetic DNA

### SEQ ID NO: 55—Description of Artificial Sequence: Synthetic DNA

### SEQ ID NO: 76—Description of Artificial Sequence: Synthetic DNA

### SEQ ID NO: 77—Description of Artificial Sequence: Synthetic DNA

### SEQ ID NO: 78—Description of Artificial Sequence: Synthetic DNA

### SEQ ID NO: 79—Description of Artificial Sequence: Synthetic DNA

### SEQ ID NO: 80—Description of Artificial Sequence: Synthetic DNA

### SEQ ID NO: 81—Description of Artificial Sequence: Synthetic DNA

### SEQ ID NO: 82—Description of Artificial Sequence: Synthetic DNA

### SEQ ID NO: 83—Description of Artificial Sequence: Synthetic DNA

### SEQ ID NO: 84—Description of Artificial Sequence: Synthetic DNA

SEQ ID NO: 85—Description of Artificial Sequence: Synthetic DNA

1. A process for producing a dipeptide or a dipeptide derivative (PI) (hereinafter referred to as dipeptide or dipeptide derivative PI), which comprises:
allowing the protein shown in any of the following [1] to [7], one or more kinds of amino acids or amino acid derivatives and ATP to be present in an aqueous medium;
allowing the dipeptide or dipeptide derivative PI to form and accumulate in the aqueous medium; and
recovering the dipeptide or dipeptide derivative PI from the aqueous medium;
[1] a protein having the amino acid sequence shown in any of SEQ ID NOS: 1 to 8;
[2] a protein consisting of an amino acid sequence wherein one or more amino acid residues are deleted, substituted or added in the amino acid sequence shown in any of SEQ ID NOS: 1 to 8 and having the activity to form the dipeptide or dipeptide derivative PI from one or more kinds of amino acids or amino acid derivatives;
[3] a protein consisting of an amino acid sequence which has 65% or more homology to the amino acid sequence shown in any of SEQ ID NOS: 1 to 8 and having the activity to form the dipeptide or dipeptide derivative PI from one or more kinds of amino acids or amino acid derivatives;
[4] a protein consisting of an amino acid sequence which has 80% or more homology to the amino acid sequence shown in SEQ ID NO: 17 and having the activity to form the dipeptide or dipeptide derivative PI from one or more kinds of amino acids or amino acid derivatives;
[5] a protein having the amino acid sequence shown in SEQ ID NO: 37 or 38;
[6] a protein consisting of an amino acid sequence wherein one or more amino acid residues are deleted, substituted or added in the amino acid sequence shown in SEQ ID NO: 37 or 38 and having the activity to form the dipeptide or dipeptide derivative PI from one or more kinds of amino acids or amino acid derivatives;
[7] a protein consisting of an amino acid sequence which has 65% or more homology to the amino acid sequence shown in SEQ ID NO: 37 or 38 and having the activity to form the dipeptide or dipeptide derivative PI from one or more kinds of amino acids or amino acid derivatives;
provided that a compound wherein the amino acids selected from the following Amino acid group A are the same or different, and are bound by peptide bond is excluded from the dipeptide or dipeptide derivative PI:
Amino acid group A; L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutanoic acid, L-azaserine, L-theanine, L-4-hydroxyproline, L-3-hydroxyproline, L-ornithine, L-citrulline, L-6-diazo-5-oxo-norleucine, glycine and β-alanine.

2. A process for producing a dipeptide or a dipeptide derivative (hereinafter referred to as dipeptide or dipeptide derivative PII), which comprises:
allowing the protein shown in any of following [1] to [7], one or more kinds of amino acids or amino acid derivatives and ATP to be present in an aqueous medium;
allowing dipeptide or dipeptide derivative PI to form and accumulate in the aqueous medium;
subjecting the dipeptide or dipeptide derivative PI, as such or after recovery, to modification to form the dipeptide or dipeptide derivative PII; and
recovering the dipeptide or dipeptide derivative PII;
[1] a protein having the amino acid sequence shown in any of SEQ ID NOS: 1 to 8;
[2] a protein consisting of an amino acid sequence wherein one or more amino acid residues are deleted, substituted or added in the amino acid sequence shown in any of SEQ ID NOS: 1 to 8 and having the activity to form the dipeptide or dipeptide derivative PI from one or more kinds of amino acids or amino acid derivatives;
[3] a protein consisting of an amino acid sequence which has 65% or more homology to the amino acid sequence shown in any of SEQ ID NOS: 1 to 8 and having the activity to form the dipeptide or dipeptide derivative PI from one or more kinds of amino acids or amino acid derivatives;
[4] a protein consisting of an amino acid sequence which has 80% or more homology to the amino acid sequence shown in SEQ ID NO: 17 and having the activity to form the dipeptide or dipeptide derivative PI from one or more kinds of amino acids or amino acid derivatives;
[5] a protein having the amino acid sequence shown in SEQ ID NO: 37 or 38;
[6] a protein consisting of an amino acid sequence wherein one or more amino acid residues are deleted, substituted or added in the amino acid sequence shown in SEQ ID NO: 37 or 38 and having the activity to form the dipeptide or dipeptide derivative PI from one or more kinds of amino acids or amino acid derivatives;
[7] a protein consisting of an amino acid sequence which has 65% or more homology to the amino acid sequence shown in SEQ ID NO: 37 or 38 and having the activity to form the dipeptide or dipeptide derivative PI from one or more kinds of amino acids or amino acid derivatives;
provided that a compound wherein the amino acids selected from the Amino acid group A described in the above are the same or different, and are bound by peptide bond is excluded from the dipeptide or dipeptide derivative PII.

3. A process for producing a dipeptide or a dipeptide derivative PI, which comprises:
allowing an enzyme source and one or more kinds of amino acids or amino acid derivatives to be present in an aqueous medium, said enzyme source being a culture or a treated matter of the culture of cells having the DNA selected from the following [1] to [5];
[1] DNA having the nucleotide sequence shown in any of SEQ ID NOS: 9 to 16 and 36;
[2] DNA which hybridizes with DNA having a nucleotide sequence complementary to the nucleotide sequence shown in any of SEQ ID NOS: 9 to 16 and 36 under stringent conditions and which encodes a protein having the activity to form the dipeptide or dipeptide derivative PI from one or more kinds of amino acids or amino acid derivatives;
[3] DNA which hybridizes with DNA having a nucleotide sequence complementary to the nucleotide sequence shown in SEQ ID NO: 18 under stringent conditions and which encodes a protein having the activity to form the dipeptide or dipeptide derivative PI from one or more kinds of amino acids or amino acid derivatives;
[4] DNA having the nucleotide sequence shown in SEQ ID NO: 39 or 40;
[5] DNA which hybridizes with DNA having a nucleotide sequence complementary to the nucleotide sequence shown in SEQ ID NO: 39 or 40 under stringent conditions and which encodes a protein having the activity to form the dipeptide or dipeptide derivative PI from one or more kinds of amino acids or amino acid derivatives;
provided that a compound wherein the amino acids selected from the Amino acid group A described in the above are the same or different, and are bound by peptide bond is excluded from the dipeptide or dipeptide derivative PI.

4. A process for producing a dipeptide or a dipeptide derivative PII, which comprises:
allowing an enzyme source and one or more kinds of amino acids or amino acid derivatives to be present in an aqueous medium, said enzyme source being a culture or a treated matter of the culture of cells having the DNA selected from the following [1] to [5];
allowing dipeptide or dipeptide derivative PI to form and accumulate in the aqueous medium;
subjecting the dipeptide or dipeptide derivative PI, as such or after recovery, to modification to form the dipeptide or dipeptide derivative PII; and
recovering the dipeptide or dipeptide derivative PII;
[1] DNA having the nucleotide sequence shown in any of SEQ ID NOS: 9 to 16 and 36;
[2] DNA which hybridizes with DNA having a nucleotide sequence complementary to the nucleotide sequence shown in any of SEQ ID NOS: 9 to 16 and 36 under stringent conditions and which encodes a protein having the activity to form the dipeptide or dipeptide derivative PI from one or more kinds of amino acids or amino acid derivatives;
[3] DNA which hybridizes with DNA having a nucleotide sequence complementary to the nucleotide sequence shown in SEQ ID NO: 18 under stringent conditions and which encodes a protein having the activity to form the dipeptide or dipeptide derivative PI from one or more kinds of amino acids or amino acid derivatives;
[4] DNA having the nucleotide sequence shown in SEQ ID NO: 39 or 40;
[5] DNA which hybridizes with DNA having a nucleotide sequence complementary to the nucleotide sequence shown in SEQ ID NO: 39 or 40 under stringent conditions and which encodes a protein having the activity to form the dipeptide or dipeptide derivative PI from one or more kinds of amino acids or amino acid derivatives.

5. The process according to claim 1, wherein the amino acids or amino acid derivatives are amino acids or amino acid derivatives represented by formula (I): 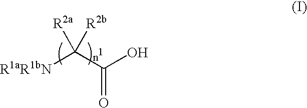
(wherein n¹ represents an integer of 1 to 3;
R^{1a} and R^{1b}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or either R^{1a} or R^{1b} may form a substituted or unsubstituted heterocyclic group together with the adjacent nitrogen atom, the carbon atom adjacent to the nitrogen atom and either R^{2a} or R^{2b} on the carbon atom; and
R^{2a} and R^{2b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{2a} or R^{2b} on the carbon atom adjacent to R^{1a}R^{1b}N may form a substituted or unsubstituted heterocyclic group together with the adjacent carbon atom, the nitrogen atom adjacent to the carbon atom and either R^{1a} or R^{1b}, and when n¹ is 2 or 3, two or three R² as and two or three R^{2b}s may be the same or different, respectively), or formula (II): 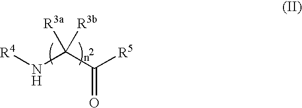
[wherein n² has the same significance as the above n¹;
R^{3a} and R^{3b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{3a} or R^{3b} on the carbon atom adjacent to R⁴HN may form a substituted or unsubstituted heterocyclic group together with the adjacent carbon atom, the nitrogen atom adjacent to the carbon atom and R⁴, and when n² is 2 or 3, two or three R³ as and two or three R^{3b}s may be the same or different, respectively;
R⁴ represents a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or R⁴ may form a substituted or unsubstituted heterocyclic group together with the adjacent nitrogen atom, the carbon atom adjacent to the nitrogen atom and either R^{3a} or R^{3b} on the carbon atom; and
R⁵ represents amino, hydroxy, substituted or unsubstituted lower alkoxy, mono(substituted or unsubstituted lower alkyl)amino, di(substituted or unsubstituted lower alkyl)amino, or an alicyclic heterocyclic group],
provided that when all the amino acids or amino acid derivatives are amino acids or amino acid derivatives represented by formula (I), at least one of R^{1a} and R^{1b} is a hydrogen atom, and when all the amino acids or amino acid derivatives are amino acids or amino acid derivatives represented by formula (II), R⁵ is hydroxy.

6. The process according to claim 1, wherein the amino acids or amino acid derivatives are amino acids or amino acid derivatives represented by formula (III): 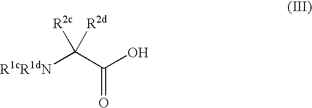
(wherein R^{1c} and R^{1d}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl; and
R^{2c} and R^{2d}, which may be the same or different, each represent a hydrogen atom or substituted or unsubstituted lower alkyl),
or formula (IV): 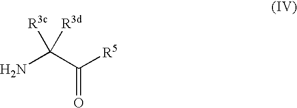
(wherein R^{3c} and R^{3d}, which may be the same or different, each represent a hydrogen atom, substituted or unsubstituted lower alkyl, substituted or unsubstituted aralkyl, or substituted or unsubstituted aryl; and
R⁵ has the same significance as defined above),
provided that when all the amino acids or amino acid derivatives are amino acids or amino acid derivatives represented by formula (II), at least one of R^{1c} and R^{1d} is a hydrogen atom, and when all the amino acids or amino acid derivatives are amino acids or amino acid derivatives represented by formula (IV), R⁵ is hydroxy.

7. The process according to claim 1, wherein the amino acids or amino acid derivatives are amino acids or amino acid derivatives represented by formula (V): 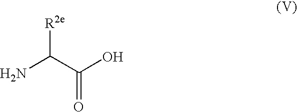
(wherein R^{2e} represents substituted or unsubstituted methyl), or formula (VI): 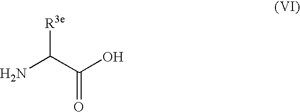
(wherein R^{3e} represents substituted or unsubstituted lower alkyl, substituted or unsubstituted aralkyl, or substituted or unsubstituted aryl).

8. The process according to claim 1, wherein the amino acids or amino acid derivatives are amino acids selected from the group consisting of L-amino acids, glycine and P-alanine, or derivatives thereof.

9. The process according to claim 8, wherein the L-amino acid is an L-amino acid selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine, L-citrulline and L-6-diazo-5-oxo-norleucine.

10. The process according to claim 1, wherein the dipeptide or dipeptide derivative PI is a dipeptide or a dipeptide derivative represented by formula (VIIa): 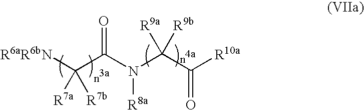
[wherein n^{3a} and n^{4a} each have the same significance as the above n¹;
R^{6a} and R^{6b}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or either R^{6a} or R^{6b} may form a substituted or unsubstituted heterocyclic group together with the adjacent nitrogen atom, the carbon atom adjacent to the nitrogen atom and either R^{7a} or R^{7b} on the carbon atom;
R^{7a} and R^{7b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{7a} or
R^{7b} on the carbon atom adjacent to R^{6a}R^{6b}N may form a substituted or unsubstituted heterocyclic group together with the adjacent carbon atom, the nitrogen atom adjacent to the carbon atom and either R^{6a} or R^{6b}, and when n^{3a} is 2 or 3, two or three R⁷ as and two or three R^{7b}s may be the same or different, respectively;
R^{8a} represents a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or R^{8a} may form a substituted or unsubstituted heterocyclic group together with the adjacent nitrogen atom, the carbon atom adjacent to the nitrogen atom and bound to R^{9a} and R^{9b}, and either R^{9a} or R^{9b} on the carbon atom;
R^{9a} and R^{9b}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{9a} or R^{9b} on the carbon atom adjacent to —R^{8a}N— may form a substituted or unsubstituted heterocyclic group together with the adjacent carbon atom, the nitrogen atom adjacent to the carbon atom and R^{8a}, and when n^{4a} is 2 or 3, two or three R⁹ as and two or three R^{9b}s may be the same or different, respectively; and
R^{10a} represents amino, hydroxy, substituted or unsubstituted lower alkoxy, mono(substituted or unsubstituted lower alkyl)amino, di(substituted or unsubstituted lower alkyl)amino, or an alicyclic heterocyclic group].

11. The process according to claim 1, wherein the dipeptide or dipeptide derivative PII is a dipeptide or a dipeptide derivative represented by formula (VIIb): 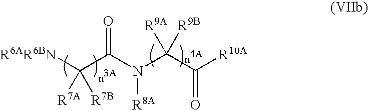
(wherein n^{3A} and n^{4A} each have the same significance as the above n¹;
R^{6A} and R^{6B}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or either R^{6A} or R^{6B} may form a substituted or unsubstituted heterocyclic group together with the adjacent nitrogen atom, the carbon atom adjacent to the nitrogen atom and either R^{7A} or R^{7B} on the carbon atom;
R^{7A} and R^{7B}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{7A} or R^{7B} on the carbon atom adjacent to R^{6A}R^{6B}N may form a substituted or unsubstituted heterocyclic group together with the adjacent carbon atom, the nitrogen atom adjacent to the carbon atom and either R^{6A} or R^{6B}, and when n^{3A} is 2 or 3, two or three R^{7A}s and two or three R^{7B}s may be the same or different, respectively;
R^{8A} represents a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl, or R^{8A} may form a substituted or unsubstituted heterocyclic group together with the adjacent nitrogen atom, the carbon atom adjacent to the nitrogen atom and bound to R^{9A} and R^{9B}, and either R^{9A} or R^{9B} on the carbon atom;
R^{9A} and R^{9B}, which may be the same or different, each represent a hydrogen atom, substituted or unsubstituted lower alkyl, substituted or unsubstituted aralkyl, substituted or unsubstituted aryl, or substituted or unsubstituted heterocyclic alkyl, or either R^{9A} or R^{9B} on the carbon atom adjacent to —R^{8A}N— may form a substituted or unsubstituted heterocyclic group together with the adjacent carbon atom, the nitrogen atom adjacent to the carbon atom and R^{8A}, and when n^{4A} is 2 or 3, two or three R^{9A}s and two or three R^{9B}s may be the same or different, respectively; and
R^{10A} has the same significance as the above R^{10a}).

12. The process according to claim 1, wherein the dipeptide or dipeptide derivative PI is a dipeptide or a dipeptide derivative represented by formula (VIIIa): 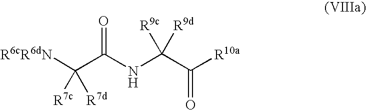
(wherein R^{6c} and R^{6d}, which may be the same or different, each represent a hydrogen atom, substituted or unsubstituted lower alkyl, substituted or unsubstituted lower alkenyl, substituted or unsubstituted lower alkynyl, substituted or unsubstituted aralkyl, substituted or unsubstituted lower alkanoyl, substituted or unsubstituted lower alkoxycarbonyl, substituted or unsubstituted aryl, or substituted or unsubstituted aroyl;
R^{7c} and R^{7d}, which may be the same or different, each represent a hydrogen atom or substituted or unsubstituted lower alkyl;
R^{9c} and R^{9d}, which may be the same or different, each represent a hydrogen atom, substituted or unsubstituted lower alkyl, substituted or unsubstituted aralkyl, or substituted or unsubstituted aryl; and
R^{10a} has the same significance as defined above).

13. The process according to claim 1, wherein the dipeptide or dipeptide derivative PII is a dipeptide or a dipeptide derivative represented by formula (VIIIb): 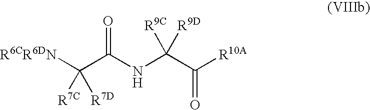
(wherein R^{6C}, R^{6D}, R^{7C}, R^{7D}, R^{9C} and R^{9D} have the same significances as the above R^{6c}, R^{6d}, R^{7c}, R^{7d}, R^{9c} and R^{9d}, respectively; and
R^{10A} has the same significance as defined above).

14. The process according to claim 1, wherein the dipeptide or dipeptide derivative PI is a dipeptide or a dipeptide derivative represented by formula (IXa): 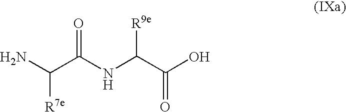
(wherein R^{7e} represents substituted or unsubstituted methyl; and
R^{9e} represents substituted or unsubstituted lower alkyl, substituted or unsubstituted aralkyl, or substituted or unsubstituted aryl).

15. The process according to claim 1, wherein the dipeptide or dipeptide derivative PII is a dipeptide or a dipeptide derivative represented by formula (IXb): 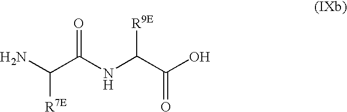
(wherein R^{7E} and R^{9E} have the same significances as the above R^{7e} and R^{9e}, respectively).

16. The process according to claim 1, wherein the dipeptide or dipeptide derivative PI or dipeptide or dipeptide derivative PII is a dipeptide or a dipeptide derivative in which the same or different amino acids or amino acid derivatives selected from the group consisting of L-amino acids, glycine, β-alanine and their derivatives are linked with each other by peptide bond.

17. The process according to claim 16, wherein the L-amino acid is an L-amino acid selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine, L-citrulline and L-6-diazo-5-oxo-norleucine.

18. The process according to claim 3, wherein the cells are cells of a microorganism.

19. The process according to claim 18, wherein the microorganism is a procaryote.

20. The process according to claim 19, wherein the procaryote is a microorganism in which the activities of one or more kinds of peptidases and one or more kinds of proteins having peptide-permeating/transporting activity (hereinafter referred to also as peptide-permeating/transporting proteins) are reduced or lost.

21. The process according to claim 20, wherein the procaryote is a microorganism in which the activities of three or more kinds of peptidases are reduced or lost.

22. The process according to claim 20, wherein the peptidase is a protein having the amino acid sequence shown in any of SEQ ID NOS: 43 to 46, or a protein having an amino acid sequence which has 80% or more homology to the amino acid sequence shown in any of SEQ ID NOS: 43 to 46 and having peptidase activity.

23. The process according to claim 20, wherein the peptide-permeating/transporting protein is a protein having the amino acid sequence shown in any of SEQ ID NOS: 47 to 51, or a protein having an amino acid sequence which has 80% or more homology to the amino acid sequence shown in any of SEQ ID NOS: 47 to 51 and having peptide-permeating/transporting activity.

24. The process according to claim 19, wherein the procaryote is a microorganism belonging to the genus Escherichia, Bacillus or Corynebacterium.

25. The process according to claim 24, wherein the microorganism belonging to the genus Escherichia, Bacillus or Corynebacterium is Escherichia coli, Corynebacterium glutamicum, Corynebacterium ammoniagenes, Corynebacterium lactofermentum, Corynebacterium flavum, Corynebacterium efficiens, Bacillus subtilis or Bacillus megaterium.

26. The process according to claim 3, wherein the treated matter of the culture is a treated matter which is selected from the group consisting of concentrated culture, dried culture, cells obtained by centrifuging the culture, dried cells, freeze-dried cells, surfactant-treated cells, ultrasonicated cells, mechanically-disrupted cells, solvent treated cells, enzymatic-treated cells, protein fractionation of cells, immobilized cells, and an enzyme preparation obtained by extracting the cells, and which has the activity to form dipeptide or dipeptide derivative from one or more kinds of amino acids or amino acid derivatives.
